SEQUENCE LISTING (34 sequences)

SEQ ID NO: 1 - protein, 159 aa, Zea mays
MATAHHADDHRAGRRSEAVGENYMRGLYGDDDYNATHYGHQQQQRPPPAPMAVAKALATATAAFSMLLLSGLAVTGTVLALIVATPLMVIFSPVLVPAAITVALLTVGIVSSGGFGVAAVAVLAWVYRYLQTTTSSSGQQPHIVKDWAQQHRLEQTRAH

SEQ ID NO: 2 - DNA, 477 bp, Zea mays
atggccacggcacaccatgctgatgatcacagagctggtcgtcgatctgaggccgtcggtgaaaattacatgagaggcctctacggagacgacgactacaatgcaacccattacggccaccaacaacaacaaagaccccctcctgcccctatggctgttgcaaaagctcttgcaactgcaaccgccgctttctcgatgctcctccttagcggtctggccgttacgggaaccgtgctcgcactgattgttgccacgcctctcatggtcatcttctcccccgtccttgtgcccgccgcaatcaccgtggccctcctgacagtcggcattgtctcgtctggcggttttggtgtcgctgctgttgcagttctcgcttgggtgtatcgttacctccaaacgacaactagcagctcgggtcagcagcctcacattgttaaagattgggcacaacagcaccgtcttgagcaaacccgtgctcac

SEQ ID NO: 3 - protein, 166 aa, Sesamum indicum
MADRDRPHPHQIQVHPQHPHRYEGGVKSLLPQKGPSTTQILAIITLLPISGTLLCLAGITLVGTLIGLAVATPVFVIFSPVLVPAAILIAGAVTAFLTSGAFGLTGLSSLSWVLNSFRRATGQGPLEYAKRGVQEGTLYVGEKTKQAGEAIKSTAKEGGREGTART

SEQ ID NO: 4 - DNA, 498 bp, Sesamum indicum
atggccgaccgtgatagacctcaccctcaccaaattcaagtgcatcctcaacacccccatagatacgagggtggtgttaagtccctcctccctcagaaaggcccctcgaccacccagattctcgctatcattacccttctccctattagcggaacactgctctgcctggccggaatcacgctggttggaaccctcatcggcctcgccgtcgcaacacctgtcttcgttattttctctcctgtgctcgtccccgcagccatcctgatcgccggtgccgttacagcatttctcacttctggcgcatttggtcttaccggcctcagctctctctcctgggtgctcaactcgttcagacgagctacgggacagggaccccttgaatacgctaagcgaggtgttcaggagggtacgctctacgttggtgagaaaacgaagcaagcaggcgaggcaatcaagtccacggccaaagaaggaggtcgagagggcactgcccgaaca

SEQ ID NO: 5 - protein, 317 aa, Limnospira maxima
MPFTIDTARSIFPETLAADVVPATIARFKQLSAEDQLALIWFAYLEMGKTITIAAPGAANMQFAENTLQEIRQMTPLQQTQAMCDLANRTDTPICRTYASWSPNIKLGFWYELGRFMDQGLVAPIPEGYKLSANANAILVTIQGIDPGQQITVLRNCVVDMGFDTSKLGSYQRVAEPVVPPQEMSQRTKVQIEGVTNSTVLQYMDNLNANDFDNLISLFAEDGALQPPFQKPIVGKENTLRFFREECQNLKLIPERGVSEPTEDGYTQIKVTGKVQTPWFGGNVGMNIAWRFLLNPENKVFFVAIDLLASPKELLNL

SEQ ID NO: 6 - DNA, 951 bp, Limnospira maxima
atgcctttcactatcgatacggctagaagcatcttccctgagacactggcagccgacgttgtccccgccacaatcgcccgatttaagcagctcagcgcagaagaccagcttgcccttatctggttcgcttacctcgagatgggaaagactatcactatcgcagcacccggagctgccaatatgcagttcgccgagaacacacttcaagagatccggcagatgactcctctccagcagacccaggccatgtgtgatctggctaatcgtaccgatactcctatttgtagaacttacgcttcttggtcccccaatattaaacttggtttctggtatgaactcggccggtttatggaccagggtcttgtggcccccattcccgaaggttacaagctctctgctaatgcaaatgcaattcttgtgaccatccaaggaattgatcctggacagcaaatcacggtcctgcggaattgcgttgttgatatgggtttcgacacatctaagctcggttcgtaccagcgggttgctgagcccgtcgttcccccccaggagatgtcccaacgaactaaggttcaaattgagggagttactaactctaccgtccttcagtacatggataatctgaacgcaaacgattttgataatcttatctcgcttttcgccgaggatggtgctcttcagcccccttttcaaaagcctatcgttggcaaagagaatacactcagatttttccgagaggaatgccagaacctcaaactcatccctgagcgtggagtttcggaacccacagaggatggatacacacagattaaggtcacgggcaaagttcagacaccctggttcggcggaaatgtgggaatgaacatcgcttggcgtttccttcttaaccctgagaacaaggttttctttgttgcaatcgatctgctcgcaagccccaaagagctcctcaacctt

SEQ ID NO: 7 - protein, 578 aa, Yarrowia lipolytica
MKLQVPAFARSSSDVKTSFMGARGQKLHNLVAAIAGLGFLLFGYDQGVMGGLLTLDTFIQQFPKMDTSDYLPPKVKTFNTTIQGTAVGIYEIGCMIGALFTMWAGDKLGRRYMIFFGSIIMTIGAILQCASYSLGQFIAGRVISGIGNGFITATVPMLQSECAKPERRGKLVMLEGALITAGIALSYWIDFGFYWVRTNDADWRFPIAFQIVFSLVLTFTIMSLPESPRWLVKKQRFEEAAGVFAALEDVPLDDPYVINQITSVKESIMMEQLAQLGVDGVDARRKIQSGEFQMGQELSFIGQMKLMFTFGKKKNFHRTMLAYWNQVMQQVTGINLITYYAAYIYQTSVGMNATDSRILAACNGTEYFMASWVAFYTIERFGRRKLMLFGAVGQACTMAILTGCVYAASKPEDGGLDMQGAGIAAAVFLFVFNTFFAIGWLGMTWLYPAEISSLEIRAPANGLSTSGNWAFNFMVVMITPVAFNSIKWKTYIIFACINAFMVPMVYFFYPETAGRSLEEIDMIFAESNPRTPWDVVGIANRLPKNSVATYDDYEAGEQEEKAIVETAESVSHDSAEFQ

SEQ ID NO: 8 - DNA, 1734 bp, Yarrowia lipolytica
atgaaactgcaagtgcccgctttcgctcggtcctcctccgacgtgaagacctctttcatgggtgcacgtggacaaaaacttcataaccttgttgccgcaatcgccggtcttggatttcttctgttcggatacgaccaaggcgtcatgggtggcctgcttacgctcgatacgttcatccaacagtttcccaaaatggatacctccgattatctgccccctaaggttaagacgtttaacacgacaattcagggaacagctgtgggaatttacgaaattggttgtatgattggcgctctcttcacaatgtgggctggtgataagctgggccgacgatacatgatcttctttggctctattatcatgaccatcggtgccattcttcagtgtgccagctattccctgggccaattcattgctggaagagtgatttctggtatcggaaacggtttcatcacggctactgtccctatgcttcagtcggaatgcgccaagcccgaaagacgtggcaaactggtgatgctggaaggagctctgatcacggcaggaattgcactcagctactggattgatttcggtttttattgggtccgaactaacgatgcagattggcggtttcctatcgcttttcagatcgtgttttctcttgttctgacttttactattatgtcccttcccgagagccccagatggctcgtcaaaaagcagcgattcgaggaggctgcaggtgtcttcgctgcacttgaggatgttccccttgatgatccctacgttatcaaccagatcacatccgtcaaagaatctatcatgatggaacagctggcccaacttggtgttgacggtgttgatgctagaagaaaaattcagagcggagagttccaaatgggtcaggagctttcttttatcggccagatgaagctgatgttcaccttcggtaagaaaaaaaattttcaccgtacaatgctggcctattggaatcaagtgatgcaacaggtgacaggtattaacctcatcacttactatgccgcctacatttaccagaccagcgttggaatgaatgccaccgactcccgtattctcgctgcctgcaatggtacggaatatttcatggcttcctgggttgccttctacacgattgaacggttcggtcgtcggaaacttatgctgttcggcgctgtgggccaagcctgcactatggctatcctcacgggctgcgtttatgcagcctccaagcccgaagatggaggcctcgacatgcaaggcgccggtattgctgcagctgttttccttttcgttttcaacacattcttcgccatcggatggcttggtatgacctggctctatcccgctgaaatttcctcgctcgagattcgagcacctgctaatggactcagcactagcggtaactgggctttcaatttcatggtcgttatgattacgcccgttgcatttaattctattaaatggaagacctacattatttttgcatgcatcaacgcatttatggtgcctatggtgtatttcttctaccccgaaactgcaggtcggtccctggaggaaattgacatgattttcgccgaatccaatccccgaacaccctgggatgtcgtcggcattgctaaccggctgcccaagaatagcgtggccacgtatgatgattacgaggctggcgagcaggaagaaaaagccatcgtcgaaactgcagagtctgtctcccacgattccgccgagtttcaa

SEQ ID NO: 9 - protein, 351 aa, Yarrowia lipolytica
MPIKDFTNPAFSNPQETSSQHTHTKMPSVADNTSNGPIEGNVLPQSKFIQHLSEYPAVAAVTGFAASFPVVKIFASNAVPLIQAIQNRGAPVAEPVVKRAAPYISQIDNAADEALNRLDKAVPSLKNTKPDEVYSRIVTQPLENVRGTVDKYADETKNTVSRVVVQPIRDVASRVQSQVVTYYDAHGKPIVHARLDPIFHPLNDRLEALINAYLPKGQEIVTDAENELARAWRLTVVAFDRARPLIEQQTSQIQEINQHTREHIQKVYDGKRSEIDDKKTVSGPVYATVATVRDLSQEGLQYAQSILNAKKPEEKADSNSGVAPVSQPHTTSAVDNSLAAPTAVHEVTASA

SEQ ID NO: 10 - DNA, 1053 bp, Yarrowia lipolytica
atgcctattaaggattttaccaatcctgcattttccaatccccaagaaacatcctctcagcacacacatacaaagatgccttcggtcgctgataatacctccaacggccccattgagggaaacgtgctgcctcaatccaagttcattcagcatctctctgagtaccctgcagttgcagcagtcacaggtttcgccgcatccttccctgttgtcaaaatttttgccagcaatgctgttccccttatccaagccattcagaaccgtggagctcccgtggcagaacctgtggtcaaacgtgcagccccttatatctcccagattgataacgctgccgacgaggctctgaatcggctggataaggcagtcccttctctcaaaaataccaaacccgatgaagtttattccagaattgtgacccaacccctggaaaacgtgcgtggcactgtggacaaatatgcagatgagacgaaaaacacagtctcccgggttgttgttcagcctatcagagatgttgcctctagagttcagtctcaggttgttacatattatgatgctcatggaaaacccatcgtgcatgcacggctcgatcccattttccacccccttaatgatagacttgaagctctgattaatgcttacctgcctaagggacaggagatcgtgacggatgccgagaacgagctcgcacgagcctggcgacttacagttgttgcattcgatcgagcccgtcctcttattgagcaacaaacctcgcagattcaagagatcaatcaacacactcgggagcatattcaaaaggtctacgacggcaaacgatccgagatcgacgacaagaagactgtttctggacctgtgtatgcaaccgtggccacggttcgggatctgtcccaagaaggacttcaatatgctcaatctattctgaacgcaaagaagcccgaagagaaagccgacagcaacagcggcgtcgcacccgtctcgcaaccccacaccacgtccgctgtggataatagccttgctgcccctactgctgtgcatgaagtcactgccagcgca

SEQ ID NO: 11 - protein, 546 aa, Petunia x hybrida
MGRKESDDGVERIEGGVVVVNPKPKKGITAKAIDLLEKVIIKLMHDSSKPLHYLSGNFAPTDETPPLNDLPIKGHLPECLNGEFVRVGPNPKFAPVAGYHWFDGDGMIHGLRIKDGKATYVSRYVRTSRLKQEEFFEGAKFMKIGDLKGLFGLFTVYMQMLRAKLKILDTSYGNGTANTALVYHHGKLLALSEADKPYALKVLEDGDLQTLGMLDYDKRLLHSFTAHPKVDPVTGEMFTFGYAHEPPYITYRVISKDGIMQDPVPITIPEAIMMHDFAITENYAIMMDLPLCFRPKEMVKNNQLAFTFDTTKKARFGVLPRYAKSEALIRWFELPNCFIFHNANAWEEGDEVVLITCRLPHPDLDMVNGEVKENLENFSNELYEMRFNMKSGAASQKKLSESSVDFPRINENYTGRKQRYVYGTTLNSIAKVTGIIKFDLHAEPETGKKQLEVGGNVQGIFDLGPGRFGSEAVFVPSQPGTECEEDDGYLIFFVHDENTGKSAVNVIDAKTMSAEPVAVVELPKRVPYGFHAFFVTEEQIQEQAKL

SEQ ID NO: 12 - DNA, 1641 bp, Petunia x hybrida
atggggcgaaaggagtcggacgacggcgttgagcgaatcgagggcggcgtggtggttgttaaccccaagcccaagaagggcatcaccgccaaggcaattgatctcctggagaaggttataattaagcttatgcacgactcctccaagcccctgcattacctgtctgggaacttcgctcccaccgacgagactccacccctgaacgatctgcccataaaggggcacctgcccgagtgtctgaacggagagttcgtgcgagtgggaccgaaccccaagttcgcccccgtggccggctatcattggttcgacggcgacgggatgatccatgggctgcgcatcaaggacggcaaggctacgtacgtatccagatacgttcgaaccagtagacttaagcaggaggagtttttcgagggcgccaagttcatgaagatcggcgacctgaaggggcttttcggactttttaccgtgtacatgcagatgcttcgagctaagctgaagatcctggatacaagctacggcaacgggaccgccaacaccgcccttgtttaccatcacgggaagctgctggctctgtctgaggccgacaagccctacgctctgaaggtgcttgaggacggcgacctccagaccctggggatgcttgactacgataagcgactgctgcactcttttaccgcacaccccaaggtggatcccgttaccggagagatgttcacattcgggtatgcccatgagccgccctatataacctaccgagtgatttccaaggacggcatcatgcaggacccggtacccattaccattcccgaggccataatgatgcacgacttcgccatcaccgagaactacgctatcatgatggaccttcccctgtgctttcgccccaaggagatggttaagaacaatcagcttgccttcacatttgacaccaccaagaaggcccggttcggcgtactgccccggtacgctaagtcggaggcacttatacgatggtttgagctgcccaactgttttattttccataatgcaaacgcctgggaggagggcgacgaggtagttctgatcacatgtcgcctgccgcaccccgatcttgatatggtaaacggcgaggtgaaggagaacctggagaactttagcaacgagctttacgagatgcgattcaatatgaagtcgggagctgcctcccagaagaagctgtccgagtcctctgtggacttcccccgcataaacgagaattataccggccgaaagcagagatacgtttacggcacaaccctgaatagcatcgcaaaggttaccggcataataaagttcgacctgcacgccgagcccgaaaccgggaagaagcagctggaggtgggcggcaacgtgcaggggatattcgatcttggccccgggcgattcggaagtgaggccgttttcgtgccatctcagcccggaaccgagtgcgaggaggacgacggctatctgattttttttgttcacgacgagaataccggcaagtccgccgttaacgttatcgacgctaagaccatgtccgctgagcccgttgctgtggtggagcttccaaagcgagtgccctacggctttcacgcatttttcgtaaccgaggagcagatccaggagcaggcaaagctttaa

SEQ ID NO: 13 - DNA, 1641 bp, Petunia x hybrida
atgggccgaaaggagtctgacgacggcgtggagcgaatcgagggcggcgtggtggtggtgaaccccaagcccaagaagggcatcaccgccaaggccatcgacctgctggagaaggtgatcatcaagctgatgcacgactcttctaagcccctgcactacctgtctggcaacttcgcccccaccgacgagactccccccctgaacgacctgcccatcaagggccacctgcccgagtgcctgaacggcgagttcgtgcgagtgggccccaaccccaagttcgcccccgtggccggctaccactggttcgacggcgacggcatgatccacggcctgcgaatcaaggacggcaaggccacctacgtgtctcgatacgtgcgaacctctcgactgaagcaggaggagttcttcgagggcgccaagttcatgaagatcggcgacctgaagggcctgttcggcctgttcaccgtgtacatgcagatgctgcgagccaagctgaagatcctggacacctcttacggcaacggcaccgccaacaccgccctggtgtaccaccacggcaagctgctggccctgtctgaggccgacaagccctacgccctgaaggtgctggaggacggcgacctccagaccctgggcatgctggactacgacaagcgactgctgcactctttcaccgcccaccccaaggtggaccccgtgaccggcgagatgttcaccttcggctacgcccacgagcccccctacatcacctaccgagtgatctctaaggacggcatcatgcaggaccccgtgcccatcaccatccccgaggccatcatgatgcacgacttcgccatcaccgagaactacgccatcatgatggacctgcccctgtgcttccgacccaaggagatggtgaagaacaaccagctggccttcaccttcgacaccaccaagaaggcccgattcggcgtgctgccccgatacgccaagtctgaggccctgatccgatggttcgagctgcccaactgcttcatcttccacaacgccaacgcctgggaggagggcgacgaggtggtgctgatcacctgccgactgccccaccccgacctggacatggtgaacggcgaggtgaaggagaacctggagaacttctctaacgagctgtacgagatgcgattcaacatgaagtctggcgccgcctctcagaagaagctgtctgagtcctctgtggacttcccccgaatcaacgagaactacaccggccgaaagcagcgatacgtgtacggcaccaccctgaactctatcgccaaggtgaccggcatcatcaagttcgacctgcacgccgagcccgagactggcaagaagcagctggaggtgggcggcaacgtgcagggcatcttcgacctgggccccggccgattcggctctgaggccgtgttcgtgccctctcagcccggcaccgagtgcgaggaggacgacggctacctgatcttcttcgtgcacgacgagaacaccggcaagtctgccgtgaacgtgatcgacgccaagaccatgtctgccgagcccgtggccgtggtggagctgcccaagcgagtgccctacggcttccacgccttcttcgtgaccgaggagcagatccaggagcaggccaagctgtaa

SEQ ID NO: 14 - protein, 543 aa, Osmanthus fragrans
MGMQGEDAQRTGNIVAVKPKPSQGLTSKAIDWLEWLFVKMMHDSKQPLHYLSGNFAPVDETPPLKDLPVTGHLPECLNGEFVRVGPNPKFASIAGYHWFDGDGMIHGMRIKDGKATYVSRYVQTSRLKQEEFFGRAMFMKIGDLKGMFGLLMVNMQMLRAKLKVLDISYGIGTANTALVYHHGKLLALSEADKPYAIKVLEDGDLQTIGLLDYDKRLAHSFTAHPKVDPFTGEMFTFGYSHTPPYVTYRVISKDGAMNDPVPITVSGPIMMHDFAITENYAIFMDLPLYFKPKEMVKDKKFIFSFDATQKARFGILPRYAKNELLIKWFELPNCFIFHNANAWEEGDEVVLITCRLENPDLDMVNSTVKERLDNFKNELYEMRFNLQNGLASQKKLSVSAVDFPRVNESYTTRKQRYVYGTTLDKIAKVTGIIKFDLHAEPETGKEKLELGGNVKGIFDLGPGRFGSEAVFVPRHPGITSEEDDGYLIFFVHDENTGKSAVNVIDAKTMSPDPVAVVELPKRVPYGFHAFFVTEDQLQEQAKV

SEQ ID NO: 15 - DNA, 1632 bp, Osmanthus fragrans
atggggatgcaaggagaggatgcgcagcggactgggaatattgttgctgtaaagccgaaacccagtcaagggctcacttccaaggccatagactggttggaatggctgtttgtgaaaatgatgcatgactccaaacagcctctccattatctttctgggaattttgctccagttgatgagactcctcctcttaaggaccttcctgttacagggcacctccctgagtgtctgaatggtgaatttgtgagggttggtcctaaccccaagtttgcttcaattgctggttatcattggtttgatggagatggaatgattcatggtatgcggataaaagatgggaaagcaacatatgtctcgcgttatgtgcagacatctcgccttaaacaagaagagttttttggcagagctatgtttatgaagattggagacctgaaagggatgtttggattgctcatggttaacatgcaaatgctcagagcaaaactgaaagtattagatatttcctatggaattggtacagctaatactgctctggtataccaccatggaaagcttttggcacttagtgaggcagataaaccatatgccattaaagttttggaagatggagatctgcaaactattggcttgctggactatgataaaagactggcacattcatttactgctcatcctaaggttgacccatttaccggagagatgtttacctttggctattcacatacaccaccttatgtcacatacagagttatatcgaaggatggagcgatgaatgatcccgttccaataacagtatcaggcccaatcatgatgcatgattttgcgattactgaaaattatgcaatttttatggacctccctttatactttaagccaaaggaaatggtgaaggataaaaagtttattttcagttttgatgctacccagaaagctcgttttggcatccttccgcggtatgcaaagaatgagctactaatcaaatggtttgagcttccaaattgcttcatattccataatgcaaatgcttgggaggaaggagatgaagttgttctgatcacttgtcgccttgagaatccagatcttgacatggttaacagcaccgttaaagaaaggcttgataatttcaaaaacgaactgtacgagatgaggttcaacctccaaaatggtctggcttcacagaaaaaactatctgtatccgctgtagattttccaagggtgaacgaaagttacactactaggaaacagcgatatgtatatggaacaacactggacaagattgctaaggtaactgggattatcaagtttgatttgcatgcggaaccagagactggaaaagaaaagctagaacttggaggaaatgttaaaggtatctttgatctaggacctggaagatttggttcagaggctgtctttgttccgcggcatcctggtatcacatctgaagaagatgatggctacttgattttctttgtacatgatgagaacactggaaagtcggcagtgaatgtaattgatgcaaaaacaatgtcacctgatcctgttgctgttgtcgaactgcccaagagagttccgtacggatttcatgccttcttcgtgacagaagaccaacttcaagaacaggcaaaggtttga

SEQ ID NO: 16 - protein, 550 aa, Zea mays
MGTEAEQPDMDSHRNDGVVVVPAPRPRKGLASWALDLLESLAVRLGHDKTKPLHWLSGNFAPVVEETPPAPNLTVRGHLPECLNGEFVRVGPNPKFAPVAGYHWFDGDGMIHAMRIKDGKATYVSRYVKTARLKQEEYFGGAKFMKIGDLKGFFGLFMVQMQQLRKKFKVLDFTYGFGTANTALIYHHGKLMALSEADKPYVVKVLEDGDLQTLGLLDYDKRLKHSFTAHPKVDPFTDEMFTFGYSHEPPYCTYRVINKEGAMLDPVPITIPESVMMHDFAITENYSIFMDLPLLFRPKEMVKNGEFIYKFDPTKKGRFGILPRYAKDDKLIRWFQLPNCFIFHNANAWEEGDEVVLITCRLENPDLDKVNGYQSDKLENFGNELYEMRFNMKTGAASQKQLSVSAVDFPRVNESYTGRKQRYVYCTILDSIAKVTGIIKFDLHAEPESGVKVLEVGGNVQGIYDLGPGRFGSEAIFVPKHPGVSGEEDDGYLIFFVHDENTGKSEVNVIDAKTMSADPVAVVELPNRVPYGFHAFFVTEDQLARQAEGQ

SEQ ID NO: 17 - DNA, 1653 bp, Zea mays
atggggacggaggcggagcagccggacatggacagccaccgaaacgacggcgtcgtggtggtgccagcgccgcgcccgcgtaaggggctcgcttcctgggcgctcgacctgcttgagtccctcgccgtgcgcctcggccacgacaagaccaagccgctccactggctctccggcaacttcgcccccgtcgtcgaggagaccccgccggccccaaaccttaccgtccgcggacacctcccggagtgcttgaatggagagtttgtcagggttgggcctaatccgaagtttgctcctgttgcggggtatcactggtttgatggagacgggatgattcatgccatgcgtattaaggatggaaaagctacctatgtatcaagatatgtgaagactgcccgcctcaaacaagaggagtattttggtggagcaaagtttatgaagattggagaccttaagggattttttggattgtttatggtccaaatgcagcaacttcggaaaaaattcaaagtcttggattttacctatggatttgggacagctaatactgcacttatatatcatcatggtaaactcatggccttgtcagaagcagataagccatatgttgttaaggtccttgaagatggagacttgcagactcttggcttgttggattatgacaaaaggttgaaacattcttttactgcccatccaaaggttgacccttttacagatgaaatgttcacattcggatattcacatgaacctccatactgtacataccgtgtgattaacaaagaaggagctatgcttgatcctgtgccaataacaataccggaatctgtaatgatgcatgattttgccatcacagagaattactctatttttatggacctccctttattgttccgaccaaaggaaatggtgaagaacggtgagtttatctacaagtttgatcctacaaagaaaggtcgttttggtattctcccccgctatgcaaaggatgacaaactcatcagatggtttcaactccctaattgtttcatattccataatgctaatgcttgggaagagggtgatgaagttgttctaattacctgccgccttgagaatccagatttggacaaggtgaatggatatcaaagtgacaagctcgaaaacttcgggaatgagctgtacgagatgagattcaacatgaaaacgggtgctgcttcacaaaagcaattgtctgtttctgctgtggattttcctcgtgttaatgagagctatactggcagaaagcagcggtatgtctactgcactatacttgacagcattgcgaaggtgactggcatcataaagtttgatctgcatgctgaaccggaaagtggtgtgaaagtacttgaagtgggaggaaatgtacaaggcatatatgacctgggacctggtagatttggttcagaggcgatttttgttcccaagcatccaggtgtgtctggagaagaagatgacggctatttgatattctttgtacacgacgagaacacagggaaatctgaagtaaatgttatcgatgcaaagacaatgtctgctgatccagttgcggtggttgagcttcctaatagggttccttatggattccatgccttttttgtaactgaggaccaactggctcgacaggcggaggggcagtga

SEQ ID NO: 18 - protein, 466 aa, Nostocales cyanobacterium
MAIKQVNPFLDGNFAPVEEEITTNTLQVIGELPPDLSGMFVRNGPNPQWTPIGQYHWFDGDGMLHGVRISNGKATYRNRYVRTKGWKIENEAGKAVWTGLLEPRPKHTPHGQSKNTANTALVWHAGQLLALWEGGAPHAIKVPELKTIGEYTYNGKLVSAVTAHPKVDPVTGEMMFFGYSFAPPYLQYSVVSPQGELLQTETIDIPMAVMMHDFAITEDYTIFMDLPLTFSQERIKRGEPLMMFERDKPSRFGIVPRYGNNSNIRWFESPACYIFHTVNAYEEGDEVVLVACRMSSTTVLGAPQDTHVDSEADIPRLHRWRFNLKTGKVSEEMLDDVPSEFPRVNENLLGQKTRYGYTGRMAKSSLPLFDGLIKYDLNNGKSQTHEFGSGRYGGEAVFVPRPGATAEDDGWLVTFVHDTAKDTSELVVVSAQDVTGEPVARVLIPQRVPYGFHGAWVSEEQLKASM

SEQ ID NO: 19 - DNA, 1401 bp, Nostocales cyanobacterium
atggcaattaagcaggtcaatcccttccttgacggtaacttcgcacccgttgaagaggagatcacaaccaacacgctgcaggtgattggagaactgccccctgacctctcgggaatgtttgttcgaaatggccctaatccccagtggacccctatcggacagtatcactggttcgacggcgatggcatgctccacggcgttcgtatttcgaacggcaaagctacttaccgtaatcggtacgttcgaacaaaaggatggaagatcgaaaacgaagctggtaaagccgtgtggaccggactccttgagccccgacctaaacacactcctcatggtcaatcgaagaacacagcaaacacagcccttgtgtggcatgcaggccaactgctggcactctgggagggcggcgcccctcacgcaatcaaagttcctgagctcaagacgatcggcgaatatacttacaatggaaaactcgtgtctgctgtcactgcccatcccaaagtggatcccgtcacgggcgagatgatgttctttggatactcgttcgcccctccttacctgcagtactctgtggtttccccccagggagaacttctgcaaacggaaaccatcgacattcctatggcagtgatgatgcacgactttgctattactgaagattatactattttcatggaccttccccttacattttctcaggagagaatcaagcgaggagagccccttatgatgtttgaaagagataagcctagccgattcggtattgtgcctcgttatggaaataactctaacattagatggttcgaatcgcccgcatgctatatcttccacacggttaatgcatacgaagaaggagatgaggttgtcctggtggcctgtcgaatgtcctctacgactgtcctgggagcacctcaagacacacatgtcgattctgaagccgatattcccagactccatcgatggcggttcaatctgaagactggtaaggtgtcggaggaaatgcttgacgacgtcccctcggaatttcctcgagttaatgaaaatctcctgggtcaaaagacccgatacggttacactggtcggatggctaagtcctccctgcctctttttgacggcctcatcaagtatgacctgaacaatggcaagagccagactcatgaattcggttctggtcgatatggaggagaagccgtgtttgtgcctcgacccggtgcaactgctgaggacgacggctggctggttaccttcgtccacgatactgctaaagacacttccgaactcgtggtcgtgtcggcacaggacgtcaccggtgagcccgtcgctagagtgcttatcccccagcgagttccttacggttttcatggcgcatgggtgtctgaggagcaactgaaggcatcgatgtga

SEQ ID NO: 20 - protein, 470 aa, Calothrix sp.
MTTNTIPTTAINPYLDGNFAPVREEITTDTLQVVGQLPPDLAGMFVRNGPNPQWPPIGKYHWFDGDGMLHGVRISNGQATYRNRYIQTRGWKIEREAGKAIWTGFMEPPRMDNPYGGYKNTGNTALIWHAGQLLALNEGGAPHAMTLPELGTIGEYTYDGKLISAFTAHPKVDPVTGEMMFFGYSFAPPYLQYSQVSAAGELLQTVPIDLPTPVMMHDFAITENYTIFMDLPLTLSAERLQRGEPMLMFERDRSSRFGIVPRYGDNSNIRWFESSPCYVFHTLNAYEVGDEVVLIGCRMSSTSVLVTEDNQTDADGNIPRLHQWRFNLKTGTVREQQLDDLAAEFPRVNEDFLGRQTRYGYAGKLANSPLPLFDGVIKYDLNGETSQIHHFGQGRYGGEAVFAPRPGATHEDDGWLITFVYDEGSDTSELVVINAQDITSEPVARVIIPQRVPYGFHGTWVSEEQLMTNS

SEQ ID NO: 21 - DNA, 1413 bp, Calothrix sp.
atgaccacaaatacgatccccactaccgccatcaacccctatcttgacggcaatttcgcacctgtgcgggaagaaatcactacggacacccttcaagttgttggtcaactccctcctgatctggccggcatgttcgtgcggaatggtcccaatccccagtggcctcctatcggtaaatatcactggttcgatggtgacggcatgctccatggagtgcgtatctcgaatggtcaggcaacctatcggaaccgatacatccaaacccgtggatggaaaattgagcgtgaagccggcaaagctatctggactggatttatggagcctcctcgaatggacaatccttatggtggatataaaaacacgggcaatacagctctgatttggcatgcaggtcagcttctggcacttaacgaaggtggagcccctcacgctatgactcttcctgagctcggaaccatcggagaatacacctatgacggtaaactcatctccgcattcactgctcaccccaaggtggatcctgttaccggcgagatgatgttctttggatactccttcgcacctccttatcttcaatacagccaggtgtctgcagcaggcgagctccttcaaactgtgcctatcgatctccctacgcctgtgatgatgcatgactttgccatcactgagaactacacaatctttatggaccttcccctgacgctttcggcagagcgtcttcaaagaggcgaacctatgcttatgttcgaacgagatcgatcgagccggttcggtatcgttccccgttatggtgataattcgaatattcgttggtttgagtcctctccctgctacgtgtttcatacactgaatgcctatgaggtgggagacgaggtcgtgcttattggatgccggatgagctccacctccgtgcttgtgacggaggacaatcagaccgacgcagacggtaatattcccagacttcaccaatggagatttaatctcaagactggtacagttcgggagcagcagctcgacgatctcgccgccgaatttccccgagttaacgaggacttcctcggaagacaaactcggtacggctatgctggaaaactggctaactcgcccctgcctctgtttgacggagttatcaaatatgacctcaacggcgagacttcccagatccaccattttggacagggaagatacggcggcgaagccgtttttgcccccagacctggtgccacccatgaagacgacggatggctcatcacatttgtctacgacgagggatccgacacgagcgagctcgtggtcatcaacgctcaagatattacgtccgaacccgttgctcgtgtgattattccccaacgtgtgccctacggatttcacggtacttgggtcagcgaagaacagctcatgacaaacagctaa

SEQ ID NO: 22 - protein, 468 aa, Calothrix brevissima
MTSNIISTTADNPYLSGNFAPVGQEIATDSLPVLGELPPDLSGMFVRNGPNPQWPPIGNYHWFDGDGMLHCVHVSEGKATYRNRYVQTQGWKIEREAGKAVWSGLLEPPQMDNPYGAYKRTANTALIWHGGQMLALHEGSAPHAIKVPELETIGEYTYNDKLVSAFTAHPKVDPVTGEMMFFGYSFTPPYLQYSVVSAVGELLQTVPIDLPVAVMMHDFAITKNYSIFLDLPLTMRGERLQRGEPLFMFERDRPSRFGIIPRYGNNSNIRWFESEPCYIFHIYNAYEIGDEVVLLGCRMSSTTVLGDDNSSDPDANVPRLHEWRFNLKTGTVSDQRLDDIPAEFPRINENYVGLPTRYGYAGKAANTPVPLFDGLIKYDFSSGKSQTHEFGQGRFGGEAVFAPRPGATAEDDGWLITFVHDETSDTSELLVVHAQDVSSEPVARVIIPQRVPYGFHGAWISEAQLKGA

SEQ ID NO: 23 - DNA, 1407 bp, Calothrix brevissima
atgactagcaatatcatttccactacagctgataatccctacctgtccggaaatttcgctcctgtcggccaggaaatcgccacagattccctccccgtcctgggtgaactccctcccgatctgagcggtatgttcgtccggaatggacccaacccccaatggcctcctatcggaaactatcattggttcgacggagatggtatgctgcactgtgttcacgtttcggaaggaaaagccacttatagaaatcgttatgtgcagacccagggttggaaaattgagcgggaagctggaaaggctgtttggtcgggtctgctcgaacctcctcagatggacaatccttacggtgcatataaaagaaccgctaatacagcactgatttggcacggaggtcaaatgctcgcactccatgagggatctgcaccccacgctattaaggtccccgagctggaaacgatcggcgagtacacatataatgataagctggtgtcggcttttacggcacatcccaaagtggatcccgtgacgggcgagatgatgttctttggctactcctttacacccccctatctgcaatattcggttgtgagcgctgttggtgagctgcttcagactgtgcccatcgatctccccgtcgccgttatgatgcatgactttgccatcactaaaaactacagcattttcctggatcttcctcttactatgcgtggagagcgtcttcaacggggagaacctctttttatgtttgagcgggaccgtccttctcgatttggtattatccctcgttatggtaataattctaatattcgttggtttgaaagcgaaccttgctatattttccacatctacaacgcttatgaaatcggagacgaggtcgtgctcctgggttgtcgaatgagctctaccaccgttctgggagatgacaactcgtcggaccccgacgccaatgttcctcgacttcatgagtggagatttaatctgaagacgggaacggtgtctgatcaacgacttgacgacattcctgctgaattccctcgaatcaacgagaattatgtcggcctccccaccagatatggctacgcaggaaaggccgcaaacacgcctgtccccctcttcgatggacttattaagtatgatttttcgtcgggtaagagccaaacccatgaatttggacagggccgttttggcggcgaagctgtcttcgcacccagacctggtgctactgctgaagatgatggatggctcatcacctttgtccacgacgaaacgtctgacacctccgaacttcttgtcgttcacgctcaggatgtgtcttctgagcccgttgcccgagtcatcattcctcagcgggtcccctacggtttccacggagcatggatttctgaagctcaactgaaaggagcctaa

SEQ ID NO: 24 - protein, 466 aa, Scytonema millei
MATTQVNPYLDGNFAPVREESTANTLQVIGELPPDLSGMFVRNGPNPQWTPIGKYHWFDGDGMLHGVRISNGKATYRNRYVRTKKWKIENEAGKALLSGLLEPPQKKNPPGASKNTANTALVWHAGQLLALWEGGAPHAIRVPELKTKGEYTYNGKLASAFTAHPKVDPVTGEMMFFGYGFAPPYLQYSVVSPEGELLQTEPIDIPMAVMMHDFAITQDYTIFMDLPLTFSQERRKRGEPMMKFERDKPSRFGIVPRYGNNSNIRWFESPACYIFHTLNAYEEGDEVVLIACRMNSTTVLDVPQDTHTDSEADIPRLHRWRFNLKTGKVSEEMLDDTASEFPRINENLLGQKTRYGYTGKMAKSSMPLFDGLIKYDFNTGKSQTHEFGRGRYGGEAVFVPRPGATAEDDGWLVTFVHDTVEETSELVVVSAQDITGEPVARVLIPQRVPYGFHGAWVSEEQLKASV

SEQ ID NO: 25 - DNA, 1401 bp, Scytonema millei
atggcaactacccaggttaacccctacctggacggaaacttcgcccccgtgagagaagagagcaccgctaatacactgcaggtcatcggagagcttccccctgatctctcgggcatgttcgttagaaatggtcccaaccctcaatggacacctatcggtaagtatcactggtttgacggcgatggcatgctccacggcgtgcgtatcagcaatggcaaagctacctatagaaatcggtacgttcggaccaaaaagtggaagattgagaatgaagcaggaaaagctctcctttctggccttcttgaaccccctcaaaagaaaaacccccctggcgcttccaagaacaccgccaataccgcccttgtgtggcatgccggacaacttctggctctttgggaaggtggtgcacctcacgcaattagagtccctgagctcaagacgaaaggtgagtatacgtacaatggaaagctggccagcgcctttacagcccatcccaaggttgatcctgtcacaggagagatgatgttttttggttacggctttgcccctccttatctgcaatattcggtcgtttctcccgaaggcgaactgctgcaaacagagcccattgacattcctatggccgtcatgatgcacgattttgccattacccaggattacaccatttttatggacctgccccttacgttttctcaggaacgtcggaaacgaggtgagcccatgatgaaattcgagcgggataaaccctcgcgtttcggaattgtccccagatacggaaataatagcaacatccgttggtttgaaagccctgcttgttacattttccataccctcaacgcatacgaagaaggtgacgaggtggttcttatcgcttgccggatgaactcgacaacagtgcttgatgtccctcaagacacccataccgatagcgaggctgatatcccccgactgcatcgatggcgttttaaccttaagactggaaaagtctcggaagagatgcttgatgacacggcatccgaatttcctcgaattaatgaaaaccttctgggtcagaaaacacgatatggatacaccggcaaaatggctaaaagcagcatgcctcttttcgacggcctcattaagtatgactttaacacgggtaaatcccagacacacgaatttggacgtggacgatatggtggtgaagccgtgttcgtccctcgacctggtgccaccgccgaggatgatggttggcttgtcacgtttgtccacgacactgtcgaggaaacgtcggagctcgttgtcgtgtctgcacaggacatcactggtgaacccgtggccagagtcctcattcctcagcgggttccctatggcttccatggagcttgggtgagcgaagaacagctgaaggcctcggtttga

SEQ ID NO: 26 - DNA, 1455 bp, Lactuca sativa
atgaagtgctccgcgaagtccgatcgttgcgtggtggataagcagggtatctccgtggccgacgaagaggattacgtcaaggccggaggctccgaacttttcttcgtccagatgcagcggaccaagtctatggagtcacagagcaagttgagcgaaaagctcgctcagattccaatcggaaactgcattttggatttggttgtcatcggctgcggcccagctggcctcgctttggcggcggaatcggcaaagcttggcctcaacgtgggcctgatcggtccagatctgccattcaccaacaactacggtgtgtggcaggacgagttcattggcctgggtcttgaaggctgcatcgaacactcttggaaggacaccctcgtctacctggatgacgccgatccaatccgcattggacgtgcttacggacgcgttcaccgtgacctcctgcacgaagagctgctgcgtcgctgcgttgagtccggcgtgtcttacctcagcagcaaggttgaacgcatcaccgaagcaccaaacggctactcactcatcgagtgcgaaggtaacatcaccatcccttgccgcctcgcaaccgtggcttcgggcgccgcctccggcaagttcctggagtacgagttgggcggacctcgcgtgtgcgtgcagaccgcgtacggcatcgaggttgaggtcgaaaacaacccatacgaccctgatctgatggttttcatggactaccgtgacttctcgaagcacaagcctgaatcccttgaggcaaagtacccaactttcctttacgtcatggctatgtccccaaccaagatcttcttcgaggaaacttgcctcgcttctcgcgaagctatgcctttcaacttgttgaagagcaagctgatgtcccgcctgaaggctatgggcatccgcatcacccgcacctacgaggaagaatggtcgtacatcccggtgggcggcagcctgccaaacaccgagcagaagaacctcgccttcggtgcagcagcctcgatggtgcacccagccaccggttactctgtggttcgctctctgagcgaagcaccaaactacgccgccgttattgcgaagatcctgcgccaggaccagtcaaaggagatgatttccctcggtaagtacactaacatctccaagcaggcctgggaaactctctggccacttgagcgcaagcgccagcgtgccttcttcctcttcggcttgtcacacatcgtgctcatggacctggaaggcactcgcactttcttccgtaccttcttccgcctgcctaagtggatgtggtggggcttcctgggctcttcgctttcctccaccgatttgattatcttcgctctctacatgttcgtcatcgctccgcactccttgcgtatggagctcgtccgtcacctgttgtccgatccgaccggtgccactatggtgaaggcatacctgaccatctaa

SEQ ID NO: 27 - protein, 109 aa, Escherichia coli
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 28 - DNA, 327 bp, Escherichia coli
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcg

SEQ ID NO: 29 - protein, 80 aa, Zea mays
MAVAKALATATAAFSMLLLSGLAVTGTVLALIVATPLMVIFSPVLVPAAITVALLTVGIVSSGGFGVAAVAVLAWVYRYL

SEQ ID NO: 30 - protein, 80 aa, Sesamum indicum
TQILAIITLLPISGTLLCLAGITLVGTLIGLAVATPVFVIFSPVLVPAAILIAGAVTAFLTSGAFGLTGLSSLSWVLNSF

SEQ ID NO: 31 - protein, 245 aa, Arabidopsis thaliana
MGSKTEMMERDAMATVAPYAPVTYHRRARVDLDDRLPKPYMPRALQAPDREHPYGTPGHKNYGLSVLQQHVSFFDIDDNGIIYPWETYSGLRMLGFNIIGSLIIAAVINLTLSYATLPGWLPSPFFPIYIHNIHKSKHGSDSKTYDNEGRFMPVNLELIFSKYAKTLPDKLSLGELWEMTEGNRDAWDIFGWIAGKIEWGLLYLLARDEEGFLSKEAIRRCFDGSLFEYCAKIYAGISEDKTAYY

SEQ ID NO: 32 - DNA, 738 bp, Arabidopsis thaliana
atggggtcaaagacggagatgatggagagagacgcaatggctacggtggctccctatgcgccggtcacttaccatcgccgtgctcgtgttgacttggatgatagacttcctaaaccttatatgccaagagcattgcaagcaccagacagagaacacccgtacggaactccaggccataagaattacggacttagtgttcttcaacagcatgtctccttcttcgatatcgatgataatggcatcatttacccttgggagacctactctggactgcgaatgcttggtttcaatatcattgggtcgcttataatagccgctgttatcaacctgacccttagctatgccactcttccggggtggttaccttcacctttcttccctatatacatacacaacatacacaagtcaaagcatggaagtgattcaaaaacatatgacaatgaaggaaggtttatgccggtgaatcttgagttgatatttagcaaatatgcgaaaaccttgccagacaagttgagtcttggagaactatgggagatgacagaaggaaaccgtgacgcttgggacatttttggatggatcgcaggcaaaatagagtggggactgttgtacttgctagcaagggatgaagaagggtttttgtcaaaagaagctattaggcggtgtttcgatggaagcttgttcgagtactgtgccaaaatctacgctggtatcagtgaagacaagacagcatactactaa

SEQ ID NO: 33 - protein, 245 aa, Sesamum indicum
MATHVLAAAAERNAALAPDAPLAPVTMERPVRTDLETSIPKPYMARGLVAPDMDHPNGTPGHVHDNLSVLQQHCAFFDQDDNGIIYPWETYSGLRQIGFNVIASLIMAIVINVALSYPTLPGWIPSPFFPIYLYNIHKAKHGSDSGTYDTEGRYLPMNFENLFSKHARTMPDRLTLNLFSKHARTMPDRLTLFGWIASKMEWTLLYILARDQDGFLSKEAIRRCYDGSLFEYCAKMQRGAEDKMK

SEQ ID NO: 34 - DNA, 738 bp, Sesamum indicum
atggcaactcatgttttggctgctgcggcggagagaaatgctgcgttggcgccggacgccccgcttgctccggtgactatggagcgcccagtgcgcactgacttggagacttcgatcccgaagccctatatggcaagaggattggttgcacctgatatggatcaccccaacggaacaccaggccatgtgcatgataatttgagtgtgctgcaacagcattgtgctttctttgatcaggatgataacggaatcatctatccatgggagacttactctggacttcgccaaattggtttcaatgtgatagcttcccttataatggctatcgtcattaatgtggcgctgagttatcctactctcccgggttggattccttctccttttttccccatatatttgtacaacatacacaaggccaaacatggaagcgactccggaacctatgatactgaaggaaggtacctacctatgaattttgagaacctgttcagcaagcatgcccggacaatgcccgataggctcactctaggggagctatggagcatgactgaagctaacagagaagcatttgacattttcggctggatcgcaagcaaaatggagtggactctcctctacattcttgcaagagaccaggacggtttcctgtcgaaagaagccatcaggcggtgttacgatggcagtttgttcgagtactgtgcaaagatgcaaaggggagccgaggacaagatgaaatga

(19) United States
(12) Patent Application Publication
(10) Pub. No.: US 2022/0325313 A1
(43) Pub. Date: Oct. 13, 2022

(54) BIOSYNTHESIS OF ALPHA-IONONE AND BETA-IONONE

(71) Applicant: Conagen Inc., Bedford, MA (US)

(72) Inventors: Yisheng Wu, Bedford, MA (US); Wanli Lu, Bedford, MA (US); Jacob Thomas Courtney, Milford, MA (US); David Nunn, Lowell, MA (US); Oliver Yu, Lexington, MA (US)

(73) Assignee: Conagen Inc., Bedford, MA (US)

(21) Appl. No.: 17/712,452

(22) Filed: Apr. 4, 2022

Related U.S. Application Data
(63) Continuation of application No. PCT/US2020/054323, filed on Oct. 5, 2020.
(60) Provisional application No. 62/911,116, filed on Oct. 4, 2019.

(51) Int. Cl. C12P 23/00; C12P 5/00; C12N 1/16; C12N 15/81; C12N 15/62; C12N 15/52

(52) U.S. Cl. CPC C12P 23/00; C12P 5/007; C12N 1/16; C12N 15/815; C12N 15/625; C12N 15/52; C12Y 113/11071; C12Y 114/13129; C12N 2800/102

(57) ABSTRACT

Provided herein are recombinant nucleic acid molecules, nucleic acid constructs, fusion enzymes, transformed host cells, and methods for making aroma compounds alpha-ionone or beta-ionone.

### RELATED APPLICATIONS

This application claims priority to U.S. Provisional Application No. 62/911,116, filed on Oct. 4, 2019, the content of which is incorporated by reference herein in its entirety.

### FIELD OF THE INVENTION

The field of the invention relates to methods and processes for the biosynthetic production of aroma compounds, in particular alpha-ionone and beta-ionone. More specifically, the present methods and processes make use of microbial host cells that have been transformed to include a heterologous nucleic acid that encodes a fusion enzyme having a lipid body compartmentalization signal tag domain coupled to a carotenoid cleavage dioxygenase (CCD) domain.

### BACKGROUND

Consumers demand foods, fragrances, and cosmetics that have pleasant tastes and odors. In many cases, these traits of pleasant smells and tastes are provided through derivatives of carotenoids, such as alpha-ionone and beta-ionone, that have exceptional aroma characteristics as their odor threshold is at the sub-ppb range. Current supply for alpha-ionone and beta-ionone is mainly addressed through either extraction processes from plants or chemical synthesis. As more consumers prefer natural ingredients, the market demand for ionones from natural sources has increased dramatically. However, approximately one hundred tons of raspberries, or twenty hectares of agricultural area are required to yield approximately one gram of alpha-ionone. In addition, plant extraction-based production has significant disadvantages such as weather effects on the strength and abundance of the compounds of interest, risks of plant diseases and/or poor harvests, the stability of the compound, the environmental impact of increased production, and trade restrictions. Structurally alpha-ionone has a chiral center. Natural alpha-ionone from plants (such as raspberry) is (R)-(+)-(E)-alpha-ionone. In contrast, chemically synthesized α-ionone has two isomers (R and S) (FIG. 1). The R-enantiomer has a unique and strong floral flavor and aroma, described as a violet-like, fruit-like or raspberry-like flavor, while the S-enantiomer has a woody scent similar to beta-ionone. Therefore, chemically synthesized alpha-ionone exists as a racemic mixture and contains enantiomers with two different scents. Its utility for the fragrance industry is therefore limited. Although new methods have been reported for the enantio-selective synthesis of (S)-alpha-ionone or (R)-alpha-ionone, the highest enantiomeric purity of the synthesized (R)-alpha-ionone report to date is about 97%, meaning substantial amounts of the (S)-enantiomer are still present. Moreover, chemical synthesis production can cause environmental issues, may use toxic precursors, and the production process itself may be subject to increased costs due to the costs of key starting materials.

Accordingly, there is a need in the art for novel methods to produce “natural” alpha-ionone and beta-ionone economically and reliably without the limitations posed by plant extraction and chemical synthesis.

### SUMMARY OF THE INVENTION

According to the current invention, alpha-ionone and beta-ionone can be reliably produced at a high yield by enzyme engineering and fermentation technology using microbial cell cultures such as oleaginous yeast Yarrowia lipolytica, baker's yeast Saccharomyces cerevisiae and/or Corynebacterium glutamicum. These microbial cell cultures can synthesize “natural” alpha-ionone or beta-ionone de novo in commercially significant yields. Hence, new biosynthetic methods are provided herein to reduce costs of alpha-ionone or beta-ionone production and lessen the environmental impact of large-scale cultivation and processing of natural sources from which these aroma compounds can be extracted.

More specifically, the present disclosure encompasses methods and compositions for making “natural” alpha-ionone or beta-ionone by microbial fermentation, where such methods and compositions involve the transformation of a eukaryotic cell with a heterologous nucleic acid molecule that encodes a fusion enzyme having a lipid body compartmentalization signal tag (LBT) domain coupled to a carotenoid cleavage dioxygenase (CCD) domain.

Fermentative production of alpha-ionone and beta-ionone has been hindered by the efficiency of the reaction step by which their respective precursor epsilon-carotene or beta-carotene is cleaved by a CCD enzyme. The present disclosure is based, in part, on the finding that certain compartmentalization signal tags can target lipid bodies, wherein epsilon-carotene and beta-carotene accumulate, thereby increasing the binding affinity of a CCD enzyme to such lipid bodies and thus dramatically increasing the catalytic efficiency of the CCD enzyme. By combining the overexpression of a nucleic acid molecule encoding a lipid body compartmentalization signal tag fused CCD, and the overexpression of various carotenoid precursor biosynthetic genes, the production of alpha-ionone or beta-ionone can be improved by providing much higher titers than alternative strategies such as the use of soluble tags, site-directed mutagenesis, or by creating an EC-CCD fusion enzyme alone. The present disclosure, therefore, provides economical and reliable methods for producing “natural” alpha-ionone and beta-ionone without the disadvantages associated with chemical synthesis or plant extraction.

In one aspect, the present invention relates to a recombinant microbial production host cell for producing an ionone compound, where the host cell includes at least one nucleic acid construct containing a coding sequence that encodes a fusion enzyme, where the fusion enzyme includes a first domain capable of functioning as a lipid body compartmentalization signal tag fused to a second domain having carotenoid cleavage activity. In various embodiments, the lipid body compartmentalization signal tag (LBT) can be a lipid body structural protein (e.g., oleosins), a lipid body surface protein (e.g., caleosin), an oleaginicity inducing lipid droplet protein (e.g., Oil1), an orange carotenoid-binding protein, and a membrane transporter protein (e.g. a sugar transporter such as STL1). The second domain can be a carotenoid cleavage dioxygenase (CCD) or a functional equivalent such as various carotenoid oxygenases and 9-cis-epoxycarotenoid dioxygenases.

In various embodiments, the present invention relates to a synthetic or recombinant nucleic acid molecule (i.e., an LBT-CCD fusion enzyme coding sequence) that includes a first nucleic acid sequence and a second nucleic acid sequence, where the first nucleic acid sequence and the second nucleic acid sequence together encode a fusion enzyme, where such fusion enzyme comprises a first domain capable of functioning as a lipid body compartmentalization signal tag and a second domain having carotenoid 9,10(9′,10′)-cleavage activity. In some embodiments, the lipid body compartmentalization signal tag can be selected from the group consisting of a lipid body structural protein, a lipid synthesis enzyme, a membrane protein, and an orange carotenoid binding protein. In certain embodiments, the lipid body compartmentalization signal tag can be a small lipid body structural protein, such as a lipid body structural protein that has a molecular weight of between 15 kDa and 25 kDa. For example, its molecular weight can be less than 25 kDa, less than 20 kDa, or around 17 or 18 kDa. In specific embodiments, the first domain of the fusion enzyme can include an amino acid sequence of SEQ ID NO: 29 or SEQ ID NO: 30.

In various embodiments, the lipid body compartmentalization signal tag which is coupled to CCD can be a Zea mays 16 kDa oleosin protein or a functional variant thereof that can bind to lipid bodies. For example, the tag of Zea mays 16 kDa oleosin protein can comprise an amino acid sequence having at least 80%, 85%, 90%, 95%, 96%, 97%, 98%, or 99% identity to SEQ ID NO: 1. In certain embodiments, the tag of Zea mays 16 kDa oleosin protein can comprise the amino acid sequence of SEQ ID NO: 1. In other embodiments, the tag of Zea mays 16 kDa oleosin protein can consist of the amino acid sequence of SEQ ID NO: 1. Accordingly, the first nucleic acid sequence can comprise a nucleic acid sequence having at least 80%, 85%, 90%, 95%, 97%, 98%, or 99% identity to SEQ ID NO: 2.

In various embodiments, the lipid body compartmentalization signal tag which is coupled to CCD can be a Sesamum indicum 17 kDa oleosin protein or a functional variant thereof that can bind to lipid bodies. For example, the tag of Sesamum indicum 17 kDa oleosin protein can comprise an amino acid sequence having at least 80%, 85%, 90%, 95%, 96%, 97%, 98%, or 99% identity to SEQ ID NO: 3. In certain embodiments, the tag of Sesamum indicum 17 kDa oleosin protein can comprise the amino acid sequence of SEQ ID NO: 3. In other embodiments, the tag of Sesamum indicum 17 kDa oleosin protein can consist of the amino acid sequence of SEQ ID NO: 3. Accordingly, the first nucleic acid sequence can comprise a nucleic acid sequence having at least 80%, 85%, 90%, 95%, 97%, 98%, or 99% identity to SEQ ID NO: 4.

In various embodiments, the lipid body compartmentalization signal tag which is coupled to CCD can be a Limnospira maxima orange carotenoid binding protein or a functional variant thereof that can bind to lipid bodies. For example, the tag of orange carotenoid binding protein can comprise an amino acid sequence having at least 80%, 85%, 90%, 95%, 96%, 97%, 98%, or 99% identity to SEQ ID NO: 5. In certain embodiments, the tag of orange carotenoid binding protein can comprise the amino acid sequence of SEQ ID NO: 5. In other embodiments, the tag of orange carotenoid binding protein can consist of the amino acid sequence of SEQ ID NO: 5. Accordingly, the first nucleic acid sequence can comprise a nucleic acid sequence having at least 80%, 85%, 90%, 95%, 97%, 98%, or 99% identity to SEQ ID NO: 6.

In various embodiments, the lipid body compartmentalization signal tag which is coupled to CCD can be a Yarrowia lipolytica membrane transporter Stl1p protein or a functional variant thereof that can bind to lipid bodies. For example, the tag of membrane transporter Stl1p protein can comprise an amino acid sequence having at least 80%, 85%, 90%, 95%, 96%, 97%, 98%, or 99% identity to SEQ ID NO: 7. In certain embodiments, the tag of membrane transporter Stl1p protein can comprise the amino acid sequence of SEQ ID NO: 7. In other embodiments, the tag of membrane transporter Stl1p protein can consist of the amino acid sequence of SEQ ID NO: 7. Accordingly, the first nucleic acid sequence can comprise a nucleic acid sequence having at least 80%, 85%, 90%, 95%, 97%, 98%, or 99% identity to SEQ ID NO: 8.

In various embodiments, the lipid body compartmentalization signal tag which is coupled to CCD can be a Yarrowia lipolytica lipid droplet protein Oil1p or a functional variant thereof that can bind to lipid bodies. For example, the tag of lipid droplet protein Oil1p can comprise an amino acid sequence having at least 80%, 85%, 90%, 95%, 96%, 97%, 98%, or 99% identity to SEQ ID NO: 9. In certain embodiments, the tag of lipid droplet protein Oil1p can comprise the amino acid sequence of SEQ ID NO: 9. In other embodiments, the tag of lipid droplet protein Oil1p can consist of the amino acid sequence of SEQ ID NO: 9. Accordingly, the first nucleic acid sequence can comprise a nucleic acid sequence having at least 80%, 85%, 90%, 95%, 97%, 98%, or 99% identity to SEQ ID NO: 10.

In various embodiments, the second domain having carotenoid 9,10(9′,10′)-cleavage activity can be a carotenoid 9,10(9′,10′)-cleavage dioxygenase (CCD). For example, the second domain can be a Petunia x hybrida CCD or a functional variant thereof. For example, the second or CCD domain can comprise an amino acid sequence having at least 80%, 85%, 90%, 95%, 96%, 97%, 98%, or 99% identity to SEQ ID NO: 11. In certain embodiments, the second or CCD domain can comprise the amino acid sequence of SEQ ID NO: 11. In other embodiments, the second or CCD domain can consist of the amino acid sequence of SEQ ID NO: 11. Accordingly, the second nucleic acid sequence can comprise a nucleic acid sequence having at least 80%, 85%, 90%, 95%, 97%, 98%, or 99% identity to SEQ ID NO: 12 or SEQ ID NO: 13.

In various embodiments, the second domain having carotenoid 9,10(9′,10′)-cleavage activity can be a carotenoid 9,10(9′,10′)-cleavage dioxygenase (CCD). For example, the second domain can be an Osmanthus fragrans CCD or a functional variant thereof. For example, the second or CCD domain can comprise an amino acid sequence having at least 80%, 85%, 90%, 95%, 96%, 97%, 98%, or 99% identity to SEQ ID NO: 14. In certain embodiments, the second or CCD domain can comprise the amino acid sequence of SEQ ID NO: 14. In other embodiments, the second or CCD domain can consist of the amino acid sequence of SEQ ID NO: 14. Accordingly, the second nucleic acid sequence can comprise a nucleic acid sequence having at least 80%, 85%, 90%, 95%, 97%, 98%, or 99% identity to SEQ ID NO: 15.

In various embodiments, the second domain having carotenoid 9,10(9′,10′)-cleavage activity can be a carotenoid 9,10(9′,10′)-cleavage dioxygenase (CCD). For example, the second domain can be a Zea mays CCD or a functional variant thereof. For example, the second or CCD domain can comprise an amino acid sequence having at least 80%, 85%, 90%, 95%, 96%, 97%, 98%, or 99% identity to SEQ ID NO: 16. In certain embodiments, the second or CCD domain can comprise the amino acid sequence of SEQ ID NO: 16. In other embodiments, the second or CCD domain can consist of the amino acid sequence of SEQ ID NO: 16. Accordingly, the second nucleic acid sequence can comprise a nucleic acid sequence having at least 80%, 85%, 90%, 95%, 97%, 98%, or 99% identity to SEQ ID NO: 17.

In various embodiments, the second domain having carotenoid 9,10(9′,10′)-cleavage activity can be a carotenoid oxygenase, a 9-cisepoxycarotenoid dioxygenase, or functional variants thereof, from various cyanobacteria such as, but not limited to, Nostocales cyanobacterium, Calothrix sp. Calothrix brevissima, and Scytonema millei. For example, the second or CCD domain can comprise an amino acid sequence having at least 80%, 85%, 90%, 95%, 96%, 97%, 98%, or 99% identity to SEQ ID NO: 18, SEQ ID NO: 20, SEQ ID NO: 22, or SEQ ID NO: 24. In certain embodiments, the second or CCD domain can comprise the amino acid sequence of SEQ ID NO: 18, SEQ ID NO: 20, SEQ ID NO: 22, or SEQ ID NO: 24. In other embodiments, the second or CCD domain can consist of the amino acid sequence of SEQ ID NO: 18, SEQ ID NO: 20, SEQ ID NO: 22, or SEQ ID NO: 24. Accordingly, the second nucleic acid sequence can comprise a nucleic acid sequence having at least 80%, 85%, 90%, 95%, 97%, 98%, or 99% identity to SEQ ID NO: 19, SEQ ID NO: 21, SEQ ID NO: 23, or SEQ ID NO: 25.

In various embodiments, the nucleic acid molecule can include a third nucleic acid sequence operably linked to the first nucleic acid sequence at one end and the second nucleic acid sequence at another end, where the third nucleic acid sequence encodes a linker that couples the first domain to the second domain. For example, the linker can include 3 to 30 amino acids in length. The linker can include amino acids selected from the group of glycine, serine, threonine, and combinations thereof. In some embodiments, the linker can be a glysine-serine linker. In certain embodiments, the linker can include one or more units of GGGGS. In specific embodiments, the linker can be GGGGS. In alternative embodiments, the linker can be GGGGSGGGGSGGGGSGGGGS.

Another aspect of the present invention relates to a nucleic acid construct. The nucleic acid construct can include the various embodiments of the nucleic acid molecule described above operably linked to a heterologous nucleic acid sequence. For example, the heterologous nucleic acid sequence can encode a promoter sequence. In some embodiments, the nucleic acid construct also can include a heterologous nucleic acid sequence encoding a lycopene epsilon-cyclase such as a Lactuca sativa lycopene epsilon-cyclase (EC). In some embodiments, the nucleic acid construct also can include a heterologous nucleic acid sequence encoding a phyotoene synthase (CarRP).

Yet another aspect of the present invention relates to a fusion enzyme that includes a lipid body compartmentalization signal tag (LBT) domain coupled to a carotenoid cleavage dioxygenase (CCD) domain.

In various embodiments, the LBT domain can be a Zea mays 16 kDa oleosin protein or a functional variant thereof that can bind to lipid bodies. For example, the LBT domain can comprise an amino acid sequence having at least 80%, 85%, 90%, 95%, 96%, 97%, 98%, or 99% identity to SEQ ID NO: 1. In certain embodiments, the LBT domain can comprise the amino acid sequence of SEQ ID NO: 1. In other embodiments, the LBT domain can consist of the amino acid sequence of SEQ ID NO: 1.

In various embodiments, the LBT domain can be a Sesamum indicum 17 kDa oleosin protein or a functional variant thereof that can bind to lipid bodies. For example, the LBT domain can comprise an amino acid sequence having at least 80%, 85%, 90%, 95%, 96%, 97%, 98%, or 99% identity to SEQ ID NO: 3. In certain embodiments, the LBT domain can comprise the amino acid sequence of SEQ ID NO: 3. In other embodiments, the LBT domain can consist of the amino acid sequence of SEQ ID NO: 3.

In various embodiments, the LBT domain can be a Limnospira maxima orange carotenoid-binding protein or a functional variant thereof that can bind to lipid bodies. For example, the LBT domain can comprise an amino acid sequence having at least 80%, 85%, 90%, 95%, 96%, 97%, 98%, or 99% identity to SEQ ID NO: 5. In certain embodiments, the LBT domain can comprise the amino acid sequence of SEQ ID NO: 5. In other embodiments, the LBT domain can consist of the amino acid sequence of SEQ ID NO: 5.

In various embodiments, the LBT domain can be a Yarrowia lipolytica membrane transporter or a functional variant thereof that can bind to lipid bodies. For example, the LBT domain can comprise an amino acid sequence having at least 80%, 85%, 90%, 95%, 96%, 97%, 98%, or 99% identity to SEQ ID NO: 7. In certain embodiments, the LBT domain can comprise the amino acid sequence of SEQ ID NO: 7. In other embodiments, the LBT domain can consist of the amino acid sequence of SEQ ID NO: 7.

In various embodiments, the LBT domain can be a Yarrowia lipolytica lipid droplet protein or a functional variant thereof that can bind to lipid bodies. For example, the LBT domain can comprise an amino acid sequence having at least 80%, 85%, 90%, 95%, 96%, 97%, 98%, or 99% identity to SEQ ID NO: 9. In certain embodiments, the LBT domain can comprise the amino acid sequence of SEQ ID NO: 9. In other embodiments, the LBT domain can consist of the amino acid sequence of SEQ ID NO: 9.

In various embodiments, the CCD domain can be an Petunia x hybrida CCD or a functional variant thereof. For example, the CCD domain can comprise an amino acid sequence having at least 80%, 85%, 90%, 95%, 96%, 97%, 98%, or 99% identity to SEQ ID NO: 11. In certain embodiments, the CCD domain can comprise the amino acid sequence of SEQ ID NO: 11. In other embodiments, the CCD domain can consist of the amino acid sequence of SEQ ID NO: 11.

In various embodiments, the CCD domain can be an Osmanthus fragrans CCD or a functional variant thereof. For example, the CCD domain can comprise an amino acid sequence having at least 80%, 85%, 90%, 95%, 96%, 97%, 98%, or 99% identity to SEQ ID NO: 14. In certain embodiments, the CCD domain can comprise the amino acid sequence of SEQ ID NO: 14. In other embodiments, the CCD domain can consist of the amino acid sequence of SEQ ID NO: 14.

In various embodiments, the CCD domain can be an Zea mays CCD or a functional variant thereof. For example, the CCD domain can comprise an amino acid sequence having at least 80%, 85%, 90%, 95%, 96%, 97%, 98%, or 99% identity to SEQ ID NO: 16. In certain embodiments, the CCD domain can comprise the amino acid sequence of SEQ ID NO: 16. In other embodiments, the CCD domain can consist of the amino acid sequence of SEQ ID NO: 16.

In various embodiments, the CCD domain can be a carotenoid oxygenase, a 9-cisepoxycarotenoid dioxygenase, or functional variants thereof, from various cyanobacteria such as, but not limited to, Nostocales cyanobacterium, Calothrix sp. Calothrix brevissima, and Scytonema millei. For example, the CCD domain can comprise an amino acid sequence having at least 80%, 85%, 90%, 95%, 96%, 97%, 98%, or 99% identity to SEQ ID NO: 18, SEQ ID NO: 20, SEQ ID NO: 22, or SEQ ID NO: 24. In certain embodiments, the CCD domain can comprise the amino acid sequence of SEQ ID NO: 18, SEQ ID NO: 20, SEQ ID NO: 22, or SEQ ID NO: 24. In other embodiments, the CCD domain can consist of the amino acid sequence of SEQ ID NO: 18, SEQ ID NO: 20, SEQ ID NO: 22, or SEQ ID NO: 24.

In any of the embodiments of the fusion enzymes described above, the LBT domain can be coupled to the CCD domain via a linker. For example, the linker can include 3 to 30 amino acids in length. The linker can include amino acids selected from the group of glycine, serine, threonine, and combinations thereof. In some embodiments, the linker can be a glysine-serine linker. In certain embodiments, the linker can include one or more units of GGGGS. In specific embodiments, the linker can be GGGGS. In alternative embodiments, the linker can be GGGGSGGGGSGGGGSGGGGS.

Yet another aspect of the present invention can relate to a method of transforming a host cell. The method can include introducing into a host cell any of the present nucleic acid molecules or nucleic acid constructs described above, and selecting or screening for a transformed host cell. The host cell can be a prokaryotic cell or a eukaryotic cell. In some embodiments, the host cell can be microbial cell such as a bacterial cell, a yeast cell, an algal cell, or a fungal cell. In other embodiments, the host cell can be a plant cell that do not naturally produce the ionones of interest. In certain embodiments, the host cell can be selected from Yarrowia, Escherichia; Salmonella; Bacillus; Acinetobacter; Streptomyces; Corynebacterium; Methylosinus; Methylomonas; Rhodococcus; Pseudomonas; Rhodobacter; Synechocystis; Saccharomyces; Zygosaccharomyces; Kluyveroumyces; Candida; Hansenula; Debaryomyces; Mucor; Pichia; Torulopsis; Aspergillus; Arthrobotlys; Brevibacteria; Microbacterium; Arthrobacter; Citrobacter; Klebsiella; Pantoea; and Clostridium. In particular embodiments, the host cell can be Yarrowia lipolytica.

Yet another aspect of the present invention can relate to a recombinant cell that includes any of the present nucleic acid molecules described above. The recombinant cell can be a prokaryotic cell or a eukaryotic cell. In some embodiments, the recombinant cell can be microbial cell such as a bacterial cell, a yeast cell, an algal cell, or a fungal cell. In other embodiments, the recombinant cell can be a plant cell that do not naturally produce the ionones of interest. In certain embodiments, the recombinant cell can be selected from Yarrowia, Escherichia; Salmonella; Bacillus; Acinetobacter; Streptomyces; Corynebacterium; Methylosinus; Methylomonas; Rhodococcus; Pseudomonas; Rhodobacter; Synechocystis; Saccharomyces; Zygosaccharomyces; Kluyveroumyces; Candida; Hansenula; Debaryomyces; Mucor; Pichia; Torulopsis; Aspergillus; Arthrobotlys; Brevibacteria; Microbacterium; Arthrobacter; Citrobacter; Klebsiella; Pantoea; and Clostridium. In particular embodiments, the recombinant cell can be Yarrowia lipolytica.

Yet another aspect of the present invention can relate to a method for producing an ionone compound such as alpha-ionone or beta-ionone. The method can include culturing a recombinant host cell according to the present teachings in a suitable medium (e.g., a medium including glucose or glycerol) under conditions whereby an ionone compound is produced. The recombinant host cell can include any of the present nucleic acid molecules or constructs described above, and where the culturing step leads to the expression of a fusion enzyme according to the present teachings, resulting in the increased production of alpha-ionone or beta-ionone by the recombinant host cell. In some embodiments, the recombinant host cell can be further transformed to overexpress one or more genes involved in the mevalonate pathway. For example, to increase production of lycopene, a precursor to the production of both alpha-ionone and beta-ionone, the transformed host cell can be transformed to overexpress one or more of hydroxymethylglutaryl-CoA synthase (HMGS), hydroxymethylglutaryl-CoA reductase (HMGR), isopentenyl diphosphate isomerase (IPI), farnesyl diphosphate synthase (FPPS), and geranylgeranyl diphosphate synthase (GGPPS). In some embodiments, the transformed host cell can be transformed to overexpress a synthetic nucleic acid molecule that encodes a fusion enzyme including an FPPS fused in-frame to a GGPPS (FPPS::GGPPS). In some embodiments, the transformed host cell can be further transformed to overexpress a phytoene dehydrogenase (CarB), a bifunctional lycopene cyclase/phytoene synthase (CarRP) or a mutant thereof (e.g., the mutant CarRP-E78K (CarRP*), where the mutation knocks out the lycopene cyclase activity of the CarRP enzyme). For example, to increase the production of beta-carotene, the immediate precursor to beta-ionone, the transformed host cell can be transformed to overexpress CarRP and CarB, which in turn increase the production of beta-ionone. Alternatively, to increase the production of epsilon-carotene, the transformed host cell can be transformed to overexpress CarRP*, CarB, and an epsilon-cyclase (EC) gene. For example, the epsilon-cyclase gene can be a Lactuca sativa EC gene. In such embodiments, the transformed host cell can be transformed to overexpress a nucleic acid molecule having a nucleic acid sequence having at least 80%, 85%, 90%, 95%, 96%, 97%, 98%, or 99% identity to SEQ ID NO: 26. In certain embodiments, the transformed host cell can be transformed to overexpress a nucleic acid molecule that encodes a fusion enzyme having a first EC domain and a second CCD domain, where the EC domain is coupled to the CCD domain via a linker according to the present teachings, to further improve the production of epsilon-carotene, and in turn, alpha-ionone.

The present teachings also encompass a recombinant host cell for producing a carotenoid compound. The host cell can include at least one nucleic acid construct that includes a coding sequence encoding a fusion enzyme having an LBT domain (e.g., an oleosin polypeptide) fused to a beta-carotene ketolase (BKT or CrtW) or a beta-carotene hydroxylase (CrtR-B). In some embodiments, the host cell can include both a coding sequence encoding an LBT-BKT fusion enzyme and a coding sequence encoding an LBT-CrtR-B fusion enzyme. The beta-carotene ketolase and the beta-carotene hydroxylase can be from Haematococcus pluvialis, Chlorella zofingiensis, Chlamydomonas reinhardtii, Paracoccus sp., and Pantoea ananatis. In one embodiment, the present invention relates to a method of producing astaxanthin which encompasses culturing a recombinant host cell that has been transformed with both a coding sequence encoding an LBT-BKT fusion enzyme and a coding sequence encoding an LBT-CrtR-B fusion enzyme under conditions whereby astaxanthin is produced. In another embodiment, the present invention relates to a method of producing canthaxanthin, where the method includes culturing a recombinant host cell that has been transformed with a coding sequence encoding an LBT-CrtW fusion enzyme under conditions whereby canthaxanthin is produced.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 shows the chemical structures of (R)-alpha-ionone (top), (S)-alpha-ionone (center), and beta-ionone (bottom).

FIG. 2 illustrates the biosynthesis pathway to alpha-ionone and beta-ionone according to the present teachings. The top portion illustrates the mevalonate pathway which can take place endogenously in a selected host cell. The bottom portion illustrates the heterologous biosynthetic pathway from geranylgeranyl diphosphate (GGPP) to phytoene to lycopene, which depending on whether an epsilon-cyclase (EC) gene or a bifunctional lycopene cyclase/phytoene synthase (CarRP) is overexpressed, leads to either epsilon-carotene or beta-carotene, respectively. In the presence of the LBT-CCD fusion enzyme according to the present teachings, epsilon-carotene is converted to alpha-ionone, and beta-carotene is converted to beta-ionone. Genes shaded in gray are those that are overexpressed in the transformed host cell.

FIG. 3 shows microscope images of the accumulation of carotenoids in lipid bodies or lipid droplets in Yarrowia lipolytica (A and B) and Corynebacterium glutamicum (C).

FIG. 4 shows the plasmids the inventors have generated to overexpress the various genes highlighted in FIG. 2. These gene cassettes were integrated into the chromosome of Yarrowia lipolytica ATCC 90811 strain as described in more detail in Examples 1, 3, 5, 7 and 8.

FIG. 5 shows HPLC profile and UV spectra confirming epsilon-carotene production in cell cultures of a transformed Yarrowia lipolytica strain.

FIG. 6 shows HPLC profile and UV spectra confirming beta-carotene production in cell cultures of a transformed Yarrowia lipolytica strain.

FIG. 7 shows GC/MS spectra confirming alpha-ionone production in cell cultures of a transformed Yarrowia lipolytica strain.

FIG. 8 compares the production titer of alpha-ionone by Yarrowia lipolytica strains that have been transformed to overexpress a heterologous CCD gene (PhCCD1) using different strategies as described in Example 7.

FIG. 9 shows GC/MS spectra confirming beta-ionone production in cell cultures of a transformed Yarrowia lipolytica strain.

FIG. 10 illustrates the astaxanthin biosynthesis pathways in Haematococcus pluvialis (left) and Chlorella zofingiensis (right). Enzymes are named according to the designation of their genes. BKT, beta-carotenoid ketolase; CrtR-B, beta-carotenoid hydroxylase. Canthaxanthin can be synthesized from beta-carotene via beta-carotenoid ketolase CrtW to echinenone, and from echinenone via CrtW to canthaxanthin.

FIG. 11 shows increased production of astaxanthin (AS) and zeaxanthin (ZX) when using a recombinant microbial production strain that has been transformed with a nucleic acid construct including a coding sequence encoding an LBT-CrtRB fusion enzyme and a coding sequence encoding an LBT-BKT fusion enzyme (Oleosin::crtRB-Oleosin::BKT), compared to a strain that has been transformed with a nucleic acid construct including a coding sequence encoding a CrtRB enzyme and a coding sequence encoding a BKT enzyme (crtRB-BKT). CX stands for canthaxanthin and BC stands for beta-carotene.

FIG. 12 shows increased production of canthaxanthin (CX) when using a recombinant microbial production strain that has been transformed with a nucleic acid construct including a coding sequence encoding an LBT-CrtW fusion enzyme (Oleosin::crtW), compared to a strain that has been transformed with a nucleic acid construct including a coding sequence encoding a CrtW enzyme (crtW). BC stands for beta-carotene.

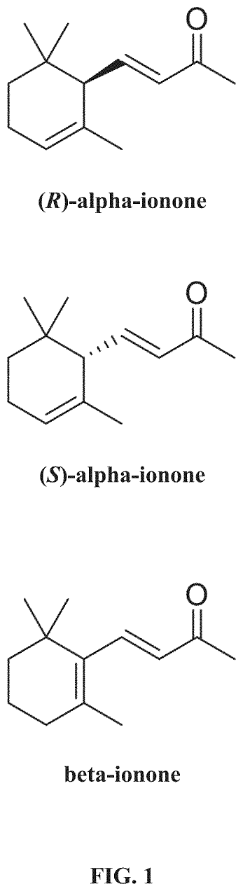

### DETAILED DESCRIPTION

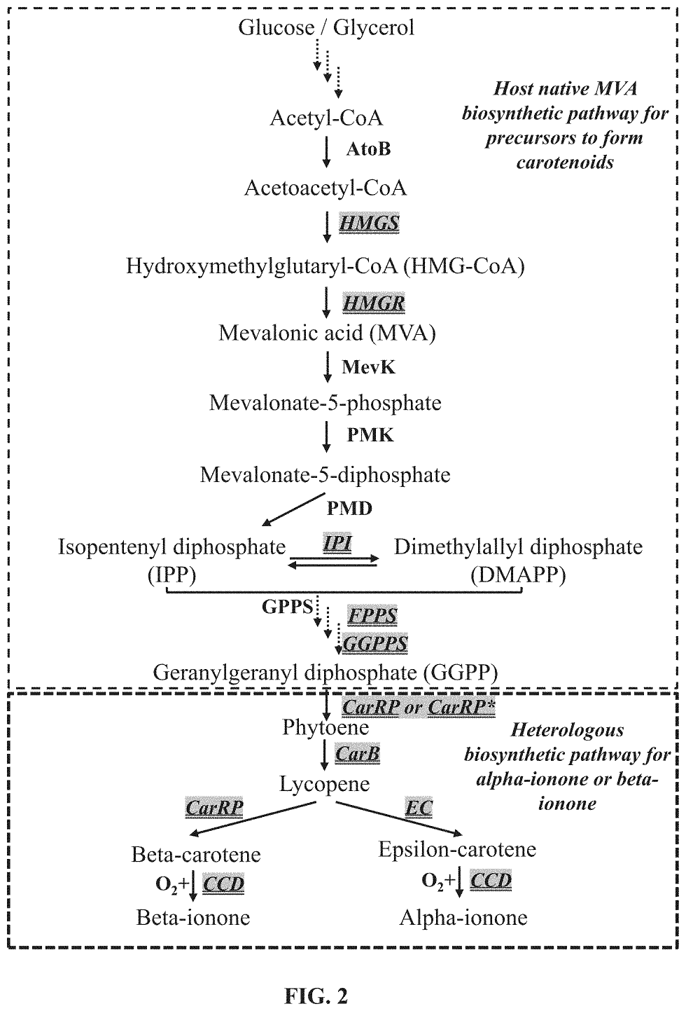

The oleaginous yeast Yarrowia lipolytica is one of the most prolific heterologous hosts for carotenoid production due to its large intercellular pool size of acetyl-CoA (the starting materials of the carotenoid backbone, see FIG. 2), and well-established genetic toolboxes. The inventors have screened a large number of combinations of different carotenoid precursor biosynthetic genes. Through co-overexpression of HMGS, HMGR, IPI, FPPS and GGPPS genes, the inventors have generated various highly efficient host strains for carotenoid production. Overexpression of CarRP*/CarB, CarRP/CarB, or carRP*/CarB/EC genes in the Yarrowia lipolytica carotenoid host strain results in high-titer production of lycopene (2 g/L), beta-carotene (4 g/L) or epsilon-carotene (1.5 g/L).

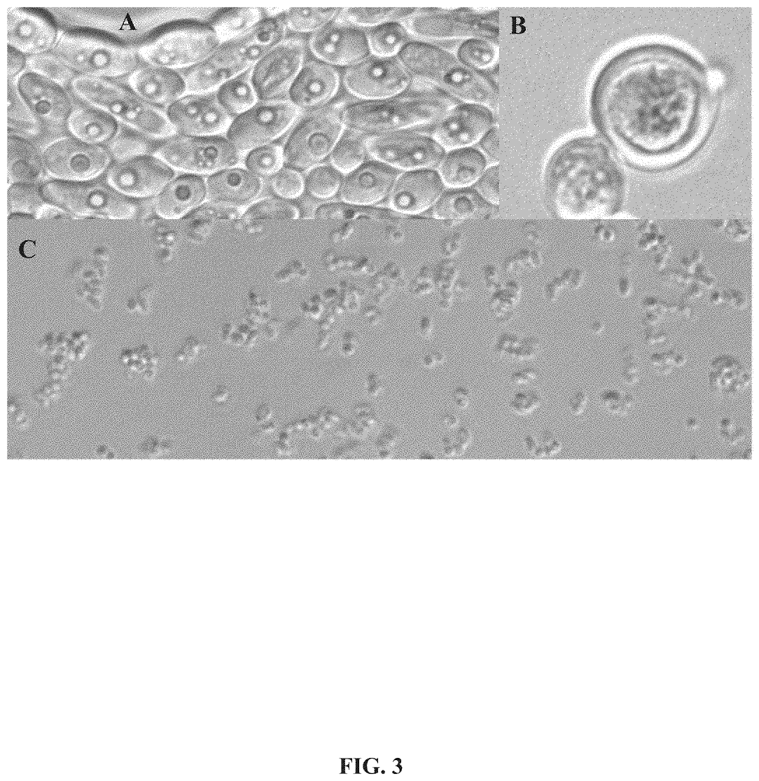

Using lycopene-producing Yarrowia lipolytica strain as a host, the inventors screened 28 CCD enzymes for ionone production. Among the various CCD enzymes that were screened, PhCCD1 (SEQ ID NO: 11) originated from the plant Petunia hybrida exhibits the highest activity. However, strains that were transformed with the wild-type PhCCD1 could only produce less than 3 mg/L of alpha-ionone from test-tube based cell cultures. It was reported that the catalytic efficiency of CCD enzyme is the key bottleneck of fermentation production of ionone compounds. The oleaginous nature of the Yarrowia lipolytica host cell is very helpful for the accumulation of carotenoids. As shown in FIGS. 3A and 3B, red-colored lycopene and orange-colored epsilon-carotene accumulate in lipid bodies in Yarrowia lipolytica cells. Similarly, lipid droplets containing carotenoids has been observed in Corynebacterium glutamicum carotene-producing strain (FIG. 3C). The inventors therefore hypothesized that the accumulation of carotene in lipid bodies (droplets) may impede the carotene substrate molecules' binding to the CCD enzyme. Therefore, besides protein engineering efforts on the PhCCD1 enzyme itself, the inventors decided to investigate a novel approach based upon lipid body based subcellular compartment engineering by screening a number of fusion enzymes that include PhCCD1 coupled to different putative compartmentalization signal tags. As the Examples show, this approach led to significant improvements in the efficiency of the carotenoid cleavage enzymes. The present invention therefore provides an economical and reliable approach for producing “natural” alpha-ionone and beta-ionone that is suitable for commercial scale-up production.

### Methods of Making Alpha-Ionone and Beta-Ionone

Methods described herein, in some embodiments, provide for the production of “natural” alpha-ionone and beta-ionone using fusion enzymes and recombinant host cells that have been transformed to express such fusion enzymes. In various embodiments, the present methods involve cellular systems that include growing cells that have been transformed with a nucleic acid molecule, where such nucleic acid molecule includes a first nucleic acid sequence and a second nucleic acid sequence, where such first and second nucleic acid sequence together encodes a heterologous fusion enzyme that includes a first domain capable of functioning as a lipid body compartmentalization signal tag (sometimes referred herein as an LBT domain) and a second domain having carotenoid 9,10(9′,10′)-cleavage activity (sometimes referred herein as a carotenoid cleavage dioxygenase (CCD) domain). In some embodiments, the cellular systems can comprise bacterial cells, yeast cells, plant cells that do not naturally produce the ionones of interest, algal cells, bacterial cells and/or fungal cells that do not naturally encode the lipid body compartmentalization signal tag and/or the CCD described herein. In particular embodiments, the cellular system can comprise growing bacteria and/or yeast cells selected from the group consisting of Yarrowia, Escherichia; Salmonella; Bacillus; Acinetobacter; Streptomyces; Corynebacterium; Methylosinus; Methylomonas; Rhodococcus; Pseudomonas; Rhodobacter; Synechocystis; Saccharomyces; Zygosaccharomyces; Kluyveroumyces; Candida; Hansenula; Debaryomyces; Mucor; Pichia; Torulopsis; Aspergillus; Arthrobotlys; Brevibacteria; Microbacterium; Arthrobacter; Citrobacter; Klebsiella; Pantoea; and Clostridium.

### Pathways and Enzymes for Biosynthesis Alpha-Ionone or Beta-Ionone

Referring to FIG. 2, biosynthetic production of alpha-ionone and beta-ionone from carbon sources like glucose or glycerol can be performed via in vivo overexpression of a series of native and heterologous genes in microbial host cells. Isopentenyl diphosphate (IPP) and its isomer dimethylallyl diphosphate (DMAPP) are the C5 building blocks for making geranylgeranyl diphosphate (GGPP), which is the direct precursor of carotenoids. In plant or fungal host cells, IPP and DMAPP can be generated from the mevalonate (MVA) pathway. Two molecules of acetyl-CoA are condensed into one molecule of acetoacetyl-CoA by acetyl-CoA acetyltransferase (AtoB). Acetoacetyl-CoA is converted into mevalonic acid via an intermediate hydroxymethylglutaryl-CoA (HMG-CoA) by HMG-CoA synthase (HMGS) and HMG-CoA reductase (HMGR), respectively. Then IPP is produced from mevalonic acid by three enzymes, mevalonate kinase (MevK), phosphomevalonate kinase (PMK) and phosphomevalonate decarboxylase (PMD). MVA pathway requires isopentenyl diphosphate isomerase (IPI) to generate DMAPP from IPP. Through the coupling of multiple IPP and DMAPP molecules, the GGPP is formed. Through the condensation of two GGPP units catalyzed by a bifunctional lycopene cyclase/phytoene synthase CarRP, phytoene is produced, which is then converted to lycopene by phytoene dehydrogenase CarB. Lycopene can be cyclized to either epsilon-carotene by epsilon-cyclase (EC), or beta-carotene by CarRP. The cleavage of epsilon-carotene or beta-carotene by a CCD enzyme results in the production of alpha-ionone or beta-ionone, respectively.

With continued reference to FIG. 2, genes that are overexpressed in the transformed microbial host cells according to the present teachings are highlighted/shaded in gray. Specifically, these include: HMGS (NCBI RefSeq XP_506052.1); HMGR (GenBank accession No. RDW25091.1); IPI (NCBI RefSeq XP_504974.1); FPPS (NCBI RefSeq XP_503599.1); GGPPS (NCBI RefSeq XP_502923.1); CarRP (UniProtKB/Swiss-Prot: Q9UUQ6.1); CarRP* (a mutant CarRP-E78K with glutamic acid mutated to lysine at position 78); CarB (GenBank accession No. OAD07725.1); EC (GenBank accession No. AAK07434.1); and PhCCD1 (GenBank accession No. AAT68189.1).

### Lipid Body Compartmentalization Signal Tags

Referring to FIG. 3, the inventors have observed accumulation of carotene substrates in lipid bodies (droplets) in Yarrowia lipolytica (A and B) and Corynebacterium glutamicum (C). Lipid bodies (droplets) are ubiquitous organelles that store metabolic energy in the form of neutral lipids such as triacylglycerols and steryl esters. Putative lipid body compartmentalization signal tags can include lipid synthesis enzymes that promote lipid body formation, and structural or membrane proteins for lipid body assembly. In addition, the orange carotenoid binding protein can be useful because of its high affinity for carotenoids. After performing rigorous screening of a large number of candidate proteins, the inventors have identified certain putative lipid body compartmentalization signal tags that have led to improvements in the in vivo carotenoid cleavage efficiency of PhCCD1 in Yarrowia lipolytica cells. These lipid body compartmentalization signal tags can include Limnospira maxima orange carotenoid binding protein, Yarrowia lipolytica membrane transporter Stl1p protein, Yarrowia lipolytica lipid droplet protein Oil1p, Zea mays 16 kDa oleosin and Sesamum indicum 17 kDa oleosin. Without wishing to be bound by any particular theory, the inventors believe that polypeptides including the amino acid sequence of SEQ ID NO: 29 or SEQ ID NO: 30, which corresponds to the conserved domain of Zea mays oleosin and Sesamum indicum oleosin, respectively, can be useful as lipid body compartmentalization signal tags according to the present teachings.

In various embodiments, the lipid body compartmentalization signal tag which is coupled to CCD can be a Zea mays 16 kDa oleosin protein or a functional variant thereof that can bind to lipid bodies. For example, the tag of Zea mays 16 kDa oleosin protein can comprise an amino acid sequence having at least 80%, 85%, 90%, 95%, 96%, 97%, 98%, or 99% identity to SEQ ID NO: 1. In certain embodiments, the tag of Zea mays 16 kDa oleosin protein can comprise the amino acid sequence of SEQ ID NO: 1. In other embodiments, the tag of Zea mays 16 kDa oleosin protein can consist of the amino acid sequence of SEQ ID NO: 1. Accordingly, the first nucleic acid sequence can comprise a nucleic acid sequence having at least 80%, 85%, 90%, 95%, 97%, 98%, or 99% identity to SEQ ID NO: 2.

In various embodiments, the lipid body compartmentalization signal tag which is coupled to CCD can be a Sesamum indicum 17 kDa oleosin protein or a functional variant thereof that can bind to lipid bodies. For example, the tag of Sesamum indicum 17 kDa oleosin protein can comprise an amino acid sequence having at least 80%, 85%, 90%, 95%, 96%, 97%, 98%, or 99% identity to SEQ ID NO: 3. In certain embodiments, the tag of Sesamum indicum 17 kDa oleosin protein can comprise the amino acid sequence of SEQ ID NO: 3. In other embodiments, the tag of Sesamum indicum 17 kDa oleosin protein can consist of the amino acid sequence of SEQ ID NO: 3. Accordingly, the first nucleic acid sequence can comprise a nucleic acid sequence having at least 80%, 85%, 90%, 95%, 97%, 98%, or 99% identity to SEQ ID NO: 4.

In various embodiments, the lipid body compartmentalization signal tag which is coupled to CCD can be a Limnospira maxima orange carotenoid binding protein or a functional variant thereof that can bind to lipid bodies. For example, the tag of orange carotenoid binding protein can comprise an amino acid sequence having at least 80%, 85%, 90%, 95%, 96%, 97%, 98%, or 99% identity to SEQ ID NO: 5. In certain embodiments, the tag of orange carotenoid binding protein can comprise the amino acid sequence of SEQ ID NO: 5. In other embodiments, the tag of orange carotenoid binding protein can consist of the amino acid sequence of SEQ ID NO: 5. Accordingly, the first nucleic acid sequence can comprise a nucleic acid sequence having at least 80%, 85%, 90%, 95%, 97%, 98%, or 99% identity to SEQ ID NO: 6.

In various embodiments, the lipid body compartmentalization signal tag which is coupled to CCD can be a Yarrowia lipolytica membrane transporter Stl1p protein or a functional variant thereof that can bind to lipid bodies. For example, the tag of membrane transporter Stl1p protein can comprise an amino acid sequence having at least 80%, 85%, 90%, 95%, 96%, 97%, 98%, or 99% identity to SEQ ID NO: 7. In certain embodiments, the tag of membrane transporter Stl1p protein can comprise the amino acid sequence of SEQ ID NO: 7. In other embodiments, the tag of membrane transporter Stl1p protein can consist of the amino acid sequence of SEQ ID NO: 7. Accordingly, the first nucleic acid sequence can comprise a nucleic acid sequence having at least 80%, 85%, 90%, 95%, 97%, 98%, or 99% identity to SEQ ID NO: 8.

In various embodiments, the lipid body compartmentalization signal tag which is coupled to CCD can be a Yarrowia lipolytica lipid droplet protein Oil1p or a functional variant thereof that can bind to lipid bodies. For example, the tag of lipid droplet protein Oil1p can comprise an amino acid sequence having at least 80%, 85%, 90%, 95%, 96%, 97%, 98%, or 99% identity to SEQ ID NO: 9. In certain embodiments, the tag of lipid droplet protein Oil1p can comprise the amino acid sequence of SEQ ID NO: 9. In other embodiments, the tag of lipid droplet protein Oil1p can consist of the amino acid sequence of SEQ ID NO: 9. Accordingly, the first nucleic acid sequence can comprise a nucleic acid sequence having at least 80%, 85%, 90%, 95%, 97%, 98%, or 99% identity to SEQ ID NO: 10.

### Carotenoid-Cleavage Domain

Enzymes that exhibit carotenoid 9,10(9′,10′)-cleavage activity can include various carotenoid 9,10(9′,10′)-cleavage dioxygenases (CCD). For example, the CCD can be a Petunia x hybrida CCD (PhCCD1) or a functional variant thereof. For example, the CCD can comprise an amino acid sequence having at least 80%, 85%, 90%, 95%, 96%, 97%, 98%, or 99% identity to SEQ ID NO: 11. In certain embodiments, the CCD can comprise the amino acid sequence of SEQ ID NO: 11. In other embodiments, the CCD can consist of the amino acid sequence of SEQ ID NO: 11. In other embodiments, the CCD can be an Osmanthus fragrans CCD or a functional variant thereof. For example, the CCD can comprise an amino acid sequence having at least 80%, 85%, 90%, 95%, 96%, 97%, 98%, or 99% identity to SEQ ID NO: 14. In certain embodiments, the CCD can comprise the amino acid sequence of SEQ ID NO: 14. In other embodiments, the CCD can consist of the amino acid sequence of SEQ ID NO: 14. In yet other embodiments, the CCD can be a Zea mays CCD or a functional variant thereof. For example, the CCD can comprise an amino acid sequence having at least 80%, 85%, 90%, 95%, 96%, 97%, 98%, or 99% identity to SEQ ID NO: 16. In certain embodiments, the CCD can comprise the amino acid sequence of SEQ ID NO: 16. In other embodiments, the CCD can consist of the amino acid sequence of SEQ ID NO: 16.

In some embodiments, the CCD can be a homolog of PhCCD1. For example, the CCD can be a carotenoid oxygenase, a 9-cisepoxycarotenoid dioxygenase, or functional variants thereof. Such homologs can originate from various cyanobacteria such as, but not limited to, Nostocales cyanobacterium, Calothrix sp. Calothrix brevissima, and Scytonema millei. For example, the CCD can comprise an amino acid sequence having at least 80%, 85%, 90%, 95%, 96%, 97%, 98%, or 99% identity to SEQ ID NO: 18, SEQ ID NO: 20, SEQ ID NO: 22, or SEQ ID NO: 24. In certain embodiments, the CCD can comprise the amino acid sequence of SEQ ID NO: 18, SEQ ID NO: 20, SEQ ID NO: 22, or SEQ ID NO: 24. In other embodiments, the CCD can consist of the amino acid sequence of SEQ ID NO: 18, SEQ ID NO: 20, SEQ ID NO: 22, or SEQ ID NO: 24.

### Fusion Enzymes

Fusion proteins including fusion enzymes have been developed as a class of novel biomolecules with multi-functional properties. By genetically fusing two or more protein domains together, the fusion protein product may obtain many distinct functions derived from each of their component moieties. The successful construction of a recombinant fusion protein requires two indispensable elements: the component proteins and the linkers. The choice of the component proteins is based on the desired functions of the fusion protein product and, according to the present teachings, includes an LBT domain and a CCD domain as described above. The selection of a suitable linker to join the protein domains together can be complicated, because an unsuitable linker can lead to misfolding of the fusion proteins, low yield in protein production, or impaired bioactivity.

In some embodiments, the linker used to couple the first LBT domain to the second CCD domain can include 3 to 30 amino acids in length. The linker can include amino acids selected from the group of glycine, serine, threonine, and combinations thereof. In some embodiments, the linker can be a glysine-serine linker. In certain embodiments, the linker can include one or more units of GGGGS. In specific embodiments, the linker can be GGGGS. In alternative embodiments, the linker can be GGGGSGGGGSGGGGSGGGGS.

### Nucleic Acids and Nucleic Acid Constructs

Nucleic acid molecules according to the present teachings include synthetic and recombinant nucleic acid molecules having a first nucleic acid sequence and a second acid sequence which together encode a fusion enzyme including an LBT domain and a CCD domain. In various embodiments, the first nucleic acid sequence can comprise a nucleic acid sequence having at least 80%, 85%, 90%, 95%, 97%, 98%, or 99% identity to SEQ ID NO: 2, SEQ ID NO: 4, SEQ ID NO: 6, SEQ ID NO: 8, or SEQ ID NO: 10. In various embodiments, the second nucleic acid sequence can comprise a nucleic acid sequence having at least 80%, 85%, 90%, 95%, 97%, 98%, or 99% identity to SEQ ID NO: 12, SEQ ID NO: 13, SEQ ID NO: 15, SEQ ID NO: 17, SEQ ID NO: 19, SEQ ID NO: 21, SEQ ID NO: 23, or SEQ ID NO: 25. In various embodiments, the present nucleic acid molecule also includes a third nucleic acid sequence that encodes a linker as described herein.

A nucleic acid construct according to the present teachings can include a synthetic or recombinant nucleic acid molecule as provided herein which can be operably linked to other heterologous nucleic acid sequences. For example, a construct as provided herein can include a nucleic acid sequence that encodes one or more fusion enzymes as described herein, in which the nucleic acid sequence further comprises a promoter operably linked to other heterologous nucleic acid sequences. In some embodiments, the heterologous nucleic acid sequences can include a regulatory element. In some embodiments, a nucleic acid sequence that encodes one or more fusion enzymes as described herein can be operably linked to a terminator sequence. In some embodiments, the nucleic acid construct is functional in a Yarrowia cell. In some embodiments, the nucleic acid construct as provided herein is further defined as an expression cassette or a vector.

### Cellular Systems

As referred herein, a cellular system according to the present methods can include any cell or cells that can be used to express the present fusion enzyme which includes an LBT domain coupled to a CCD domain. Such cellular system can include, but are not limited to, bacterial cells, yeast cells, plant cells, and animal cells. In some embodiments, the cellular system comprises bacterial cells, yeast cells, or a combination thereof. In some embodiments, the cellular system comprises prokaryotic cells, eukaryotic cells, and combinations thereof.

Bacterial cells of the present disclosure include, without limitation, Escherichia spp., Streptomyces spp., Zymomonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp., Pantoea spp., and Vibrio natriegens.

Yeast cells of the present disclosure include, without limitation, Saccharomyces spp., Schizosaccharomyces, Hansenula, Candida, Kluyveromyces, Yarrowia, Candida boidinii, and Pichia. According to the current disclosure, a yeast as claimed herein are eukaryotic, single-celled microorganisms classified as members of the fungus kingdom. Yeasts are unicellular organisms which evolved from multicellular ancestors but with some species useful for the current disclosure being those that can develop multicellular characteristics by forming strings of connected budding cells known as pseudo hyphae or false hyphae.

### Cell Culture

A cell culture refers to any cell or cells that are in a culture. Culturing or incubating is the process in which cells are grown under controlled conditions, typically outside of their natural environment. For example, cells, such as yeast cells, may be grown as a cell suspension in liquid nutrient broth. A cell culture includes, but is not limited to, a bacterial cell culture, a yeast cell culture, a plant cell culture, and an animal cell culture. In some embodiments, the cell culture comprises bacterial cells, yeast cells, or a combination thereof.

A bacterial cell culture of the present disclosure comprises bacterial cells including, but not limited to, Escherichia spp., Streptomyces spp., Zymomonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp., Pantoea spp, and Vibrio natriegens.

A yeast cell culture of the present disclosure comprises yeast cells including, but not limited to Saccharomyces spp., Schizosaccharomyces, Hansenula, Candida, Kluyveromyces, Yarrowia, Candida boidinii, and Pichia.

In some embodiments, a cell culture as described herein can be an aqueous medium including one or more nutrient substances as known in the art. Such liquid medium can include one or more carbon sources, nitrogen sources, inorganic salts, and/or growth factors. Suitable carbon sources can include glucose, fructose, xylose, sucrose, maltose, lactose, mannitol, sorbitol, glycerol, and corn syrup. Examples of suitable nitrogen sources can include organic and inorganic nitrogen-containing substances such as peptone, corn steep liquor, mean extract, yeast extract, casein, urea, amino acids, ammonium salts, nitrates and mixtures thereof. Examples of inorganic salts can include phosphates, sulfates, magnesium, sodium, calcium, and potassium salts. The liquid medium also can include one or more vitamins and/or minerals.

In some embodiments, cells are cultured at a temperature of 16° C. to 40° C. For example, cells may be cultured at a temperature of 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C. or 40° C.

In some embodiments, cells are cultured at a pH range from about 3 to about 9, preferably in the range of from about 4 to about 8. The pH can be regulated by the addition of an inorganic or organic acid or base such as hydrochloric acid, acetic acid, sodium hydroxide, calcium carbonate, ammonia, or by the addition of a buffer such as phosphate, phthalate or Tris®.

In some embodiments, cells are cultured for a period of 0.5 hours to 96 hours, or more. For example, cells may be cultured for a period of 12, 18, 24, 30, 36, 42, 48, 54, 60, 66, or 72 hours. Typically, cells, such as bacterial cells, are cultured for a period of 12 to 24 hours. In some embodiments, cells are cultured for 12 to 24 hours at a temperature of 37° C. In some embodiments, cells are cultured for 12 to 24 hours at a temperature of 16° C.

In some embodiments, cells are cultured to a density of 1×108 (OD600<1) to 2×1011 (OD˜200) viable cells/ml cell culture medium. In some embodiments, cells are cultured to a density of 1×108, 2×108, 3×108, 4×108, 5×108, 6×108, 7×108, 8×108, 9×108, 1×109, 2×109, 3×109, 4×109, 5×109, 6×109, 7×109, 8×109, 9×109, 1×1010, 2×1010, 3×1010, 4×1010, 5×1010, 6×1010, 7×1010, 8×1010, 9×1010, 1×1011, or 2×1011 viable cells/ml. (Conversion factor: OD 1=8×108 cells/ml).

### Synthetic Biology

Standard recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and Ausubel, F. M. et al., IN CURRENT PROTOCOLS IN MOLECULAR BIOLOGY, published by GREENE PUBLISHING AND WILEY-INTERSCIENCE, 1987; the entirety of each of which is hereby incorporated herein by reference.

### Microbial Production Systems

Expression of proteins in transformed host cells is most often carried out in a bacterial or yeast host cell with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such vectors are within the scope of the present disclosure.

In an embodiment, the expression vector includes those genetic elements for expression of the recombinant polypeptide in microbial cells. The elements for transcription and translation in the microbial cell can include a promoter, a coding region for the protein complex, and a transcriptional terminator.

Persons of ordinary skill in the art will be aware of the molecular biology techniques available for the preparation of expression vectors. The polynucleotide used for incorporation into the expression vector of the subject technology, as described herein, can be prepared by routine techniques such as polymerase chain reaction (PCR).

A number of molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provide are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities and fill-in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun, et al, BIOTECHNIQUES 13, 515-18 (1992), which is incorporated herein by reference to the extent it is consistent herewith).

In an embodiment, in order to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

In an embodiment, a polynucleotide for incorporation into an expression vector of the subject technology is prepared by the use of PCR using appropriate oligonucleotide primers. The coding region is amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into plant or microbial host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector,

In some embodiments, the transformed cell can be a bacterial cell, a yeast cell, an algal cell, a fungal cell, a plant cell, an insect cell or an animal cell. In some embodiments, the cell is a plant cell selected from the group consisting of: canola plant cell, a rapeseed plant cell, a palm plant cell, a sunflower plant cell, a cotton plant cell, a corn plant cell, a peanut plant cell, a flax plant cell, a sesame plant cell, a soybean plant cell, and a petunia plant cell.

Microbial host cell expression systems and expression vectors containing regulatory sequences that direct high-level expression of foreign proteins are well known to those skilled in the art. Any of these could be used to construct vectors for expression of the recombinant polypeptide of the subjection technology in a microbial host cell. These vectors could then be introduced into appropriate microorganisms via transformation to allow for high level expression of the recombinant polypeptide of the subject technology.

Vectors or cassettes useful for the transformation of suitable microbial host cells are well known in the art. Typically the vector or cassette contains sequences directing transcription and translation of the relevant polynucleotide, a selectable marker, and sequences allowing autonomous replication or chromosomal integration. Suitable vectors comprise a region 5′ of the polynucleotide which harbors transcriptional initiation controls and a region 3′ of the DNA fragment which controls transcriptional termination. It is preferred for both control regions to be derived from genes homologous to the transformed host cell, although it is to be understood that such control regions need not be derived from the genes native to the specific species chosen as a host.

Initiation control regions or promoters, which are useful to drive expression of the recombinant polypeptide in the desired microbial host cell are numerous and familiar to those skilled in the art. Virtually any promoter capable of driving these genes is suitable for the subject technology including but not limited to CYCI, HIS3, GALI, GALIO, ADHI, PGK, PH05, GAPDH, ADCI, TRPI, URA3, LEU2, ENO, TPI (useful for expression in Saccharomyces); TEF (useful for expression in Yarrowia); AOXI (useful for expression in Pichia); and lac, trp, JPL, IPR, T7, tac, and trc (useful for expression in Escherichia coli).

Termination control regions may also be derived from various genes native to the microbial hosts. A termination site optionally may be included for the microbial hosts described herein.

In plant cells, the expression vectors of the subject technology can include a coding region operably linked to promoters capable of directing expression of the recombinant polypeptide of the subject technology in the desired tissues at the desired stage of development. For reasons of convenience, the polynucleotides to be expressed may comprise promoter sequences and translation leader sequences derived from the same polynucleotide. 3′ non-coding sequences encoding transcription termination signals should also be present. The expression vectors may also comprise one or more introns in order to facilitate polynucleotide expression.

For plant host cells, any combination of any promoter and any terminator capable of inducing expression of a coding region may be used in the vector sequences of the subject technology. Some suitable examples of promoters and terminators include those from nopaline synthase (nos), octopine synthase (ocs) and cauliflower mosaic virus (CaMV) genes. One type of efficient plant promoter that may be used is a high-level plant promoter. Such promoters, in operable linkage with an expression vector of the subject technology should be capable of promoting the expression of the vector. High level plant promoters that may be used in the subject technology include the promoter of the small subunit (s) of the ribulose-1,5-bisphosphate carboxylase for example from soybean (Berry-Lowe et al., J. MOLECULAR AND APP. GEN., 1:483-98 (1982), the entirety of which is hereby incorporated herein to the extent it is consistent herewith), and the promoter of the chlorophyll binding protein. These two promoters are known to be light-induced in plant cells (see, for example, GENETIC ENGINEERING OF PLANTS, AN AGRICULTURAL PERSPECTIVE, A. Cashmore, Plenum, N.Y. (1983), pages 29-38; Coruzzi, G. et al., THE JOURNAL OF BIOLOGICAL CHEMISTRY, 258: 1399 (1983), and Dunsmuir, P. et al., JOURNAL OF MOLECULAR AND APPLIED GENETICS, 2:285 (1983), each of which is hereby incorporated herein by reference to the extent they are consistent herewith).

### Analysis of Sequence Similarity Using Identity Scoring

As used herein, “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, Mass.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this disclosure “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, JOURNAL OF MOLECULAR BIOLOGY 48:443-53, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981, Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-10 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 98% or about 99% sequence identity. Thus, one embodiment of the disclosure is a polynucleotide molecule that has at least about 70% sequence identity, at least about 80% sequence identity, at least about 85% identity, at least about 90% sequence identity, or even greater sequence identity, such as about 98% or about 99% sequence identity with a polynucleotide sequence described herein.

### Identity and Similarity

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

### Explanation of Terms Used Herein:

As used herein, the singular forms “a, an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

The term “complementary” is to be given its ordinary and customary meaning to a person of ordinary skill in the art and is used without limitation to describe the relationship between nucleotide bases that are capable to hybridizing to one another. For example, with respect to DNA, adenosine is complementary to thymine and cytosine is complementary to guanine. Accordingly, the subjection technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

“Coding sequence” is to be given its ordinary and customary meaning to a person of ordinary skill in the art, and is used without limitation to refer to a DNA sequence that encodes for a specific amino acid sequence.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing the desired product.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,” and “peptide” are to be given their respective ordinary and customary meanings to a person of ordinary skill in the art; the three terms are sometimes used interchangeably, and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

“Percent (%) amino acid sequence identity” with respect to the variant polypeptide sequences of the subject technology refers to the percentage of amino acid residues in a candidate sequence that are identical with the amino acid residues of a reference polypeptide, after aligning the sequences and introducing gaps, if necessary, to achieve the maximum percent sequence identity, and not considering any conservative substitutions as part of the sequence identity.

Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For example, the % amino acid sequence identity may be determined using the sequence comparison program NCBI-BLAST2. The NCBI-BLAST2 sequence comparison program may be downloaded from ncbi.nlm.nih.gov. NCBI BLAST2 uses several search parameters, wherein all of those search parameters are set to default values including, for example, unmask yes, strand=all, expected occurrences 10, minimum low complexity length=15/5, multi-pass e-value=0.01, constant for multi-pass=25, dropoff for final gapped alignment=25 and scoring matrix=BLOSUM62. In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

In this sense, techniques for determining amino acid sequence “similarity” are well known in the art. In general, “similarity” refers to the exact amino acid to amino acid comparison of two or more polypeptides at the appropriate place, where amino acids are identical or possess similar chemical and/or physical properties such as charge or hydrophobicity. A so-termed “percent similarity” may then be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded therein, and comparing this to a second amino acid sequence. In general, “identity” refers to an exact nucleotide to nucleotide or amino acid to amino acid correspondence of two polynucleotides or polypeptide sequences, respectively. Two or more polynucleotide sequences can be compared by determining their “percent identity”, as can two or more amino acid sequences. The programs available in the Wisconsin Sequence Analysis Package, Version 8 (available from Genetics Computer Group, Madison, Wis.), for example, the GAP program, are capable of calculating both the identity between two polynucleotides and the identity and similarity between two polypeptide sequences, respectively. Other programs for calculating identity or similarity between sequences are known by those skilled in the art.

An amino acid position “corresponding to” a reference position refers to a position that aligns with a reference sequence, as identified by aligning the amino acid sequences. Such alignments can be done by hand or by using well-known sequence alignment programs such as ClustalW2, Blast 2, etc.

Unless specified otherwise, the percent identity of two polypeptide or polynucleotide sequences refers to the percentage of identical amino acid residues or nucleotides across the entire length of the shorter of the two sequences.

The term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art, and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art, and is used without limitation to refer to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters, which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the art, and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is to be given its ordinary and customary meaning to a person of reasonable skill in the field, and is used without limitation to refer to the transfer of a polynucleotide into a target cell for further expression by that cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “recombinant” or “transformed” organisms.

The terms “transformed,” “transgenic,” and “recombinant,” when used herein in connection with host cells, are to be given their respective ordinary and customary meanings to a person of ordinary skill in the art, and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

### EXAMPLES

### Example 1: Generation of Epsilon-Carotene Producing Yarrowia lipolytica Strains

In general, the constitutive TEF promoter and the XPR2 terminator were used for the overexpression of individual genes in Yarrowia lipolytica ATCC 90811 host cells. A pUC57-Kan based vector carrying both the TEF promoter and the XPR2 terminator was cloned. Individual genes were cloned in between the TEF promoter and the XPR2 terminator using Gibson assembly. Cassettes containing the TEF promoter, one or more genes for overexpression, and the XPR2 terminator were amplified with PCR and then cloned into high copy number Yarrowia integration vectors such as pYlex-SDH, pYconVec-Leu, and pYconVec-Leu, which has auxotrophic lysine, leucine and uracil markers, respectively.

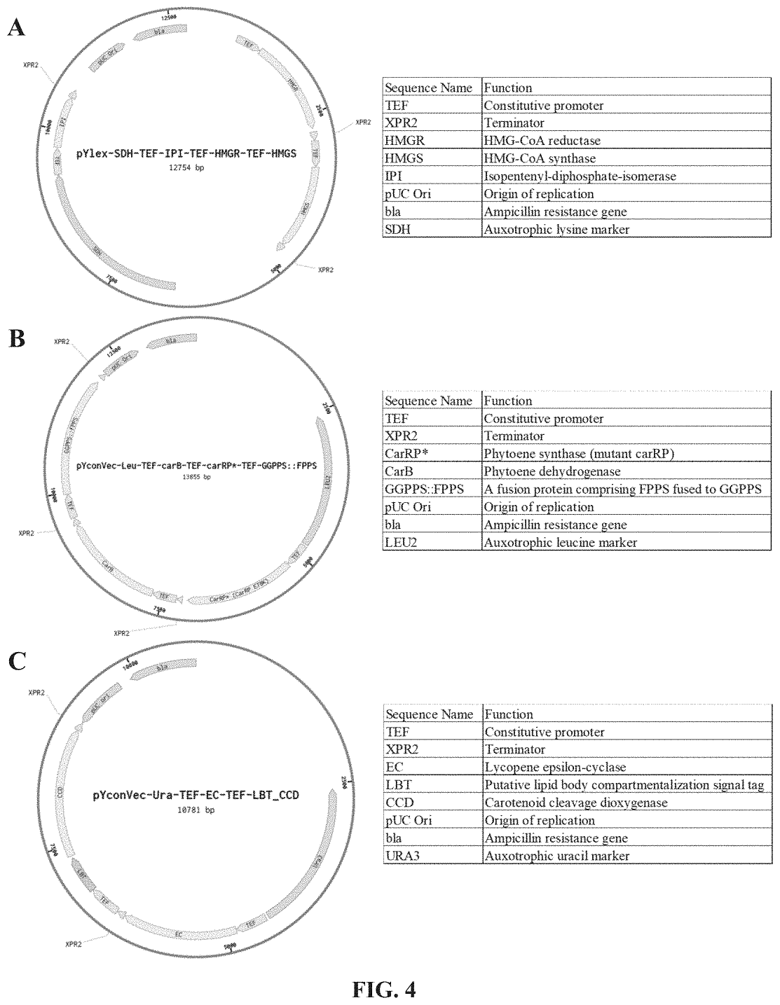

In order to boost the carotenoids precursors supply, three genes including genes encoding HMGS, HMGR and IPI, along with their own TEF promoter and XPR2 terminator, were cloned into pYlex-SDH vector to generate a pYlex-SDH-TEF-IPI-TEF-HMGR-TEF-HMGS construct (FIG. 4A). The pYlex-SDH-TEF-IPI-TEF-HMGR-TEF-HMGS construct was then used to transform Yarrowia lipolytica ATCC 90811 host and select on minimal media plate without lysine.

Two genes for lycopene production, specifically genes encoding CarRP* (a mutant CarRP that has completely lost its lycopene cyclase activity) and CarB, and a gene encoding a fusion protein comprising FPPS fused in-frame to GGPPS, along with their own TEF promoter and XPR2 terminator, were cloned into pYconVec-Leu vector to generate a pYconVec-Leu-TEF-carB-TEF-carRP*-TEF-GGPPS::FPPS construct (FIG. 4B). The pYconVec-Leu-TEF-carB-TEF-carRP*-TEF-GGPPS::FPPS construct was then used to transform Yarrowia lipolytica ATCC 90811 strain hosting pYlex-SDH-TEF-IPI-TEF-HMGR-TEF-HMGS and select on minimal media plate without leucine. The red-colored clones were Yarrowia lipolytica lycopene producers. The colony with the highest lycopene content was named LYC-40 and chosen as the host for epsilon-carotene and alpha-ionone production.

The epsilon-cyclase (EC) gene, along with a TEF promoter and an XPR2 terminator, were cloned into a pYconVec-Ura vector to generate a pYconVec-Ura-TEF-EC construct. The pYconVec-Ura-TEF-EC was then used to transform lycopene-producing Yarrowia lipolytica ATCC 90811 host LYC-40 and select on minimal media plate without uracil. Thus, the orange-colored epsilon-carotene producing Yarrowia lipolytica strain was generated.

### Example 2: Extraction of Epsilon-Carotene for HPLC Analysis

The epsilon-carotene producing Yarrowia lipolytica strain was streaked onto YPD agar plate and grown at 30° C. After 2 days of incubation, the orange-colored colonies were grown in 5 ml of YPD liquid medium at 250 rpm and at 30° C. for 4 days. 0.2 ml of cell culture was harvested by centrifugation and the cell pellet was resuspended with 0.8 ml of methanol and 0.2 ml of dichloromethane. 0.5 mm diameter glass beads were then added. The Yarrowia lipolytica cells were disrupted for 1 minute in a bead beater homogenizer. Then the mixture was centrifuged for 10 min at 15,000 rpm and the supernatant was collected for HPLC analysis.

HPLC analysis of epsilon-carotene was performed using an Ultimate 3000 HPLC System (Dionex, Sunnyvale, Calif.) that included a quaternary pump, a temperature controlled column compartment, an auto sampler and a UV absorbance detector. Gemini C18 (150 mm×4.6 mm 3 μm) with guard column was used for the characterization of lycopene, delta-carotene and epsilon-carotene. A flow rate of 1.0 ml/min was applied, and the mobile phase was composed of isopropanol (A) and acetonitrile (B). The program is 0-5 min 85% B; 5-13 min 85%-60% B; 13-14 min 60%-85% B; 14-16 min 85% B. The detection wavelengths were 474 nm for lycopene, delta-carotene and epsilon-carotene.

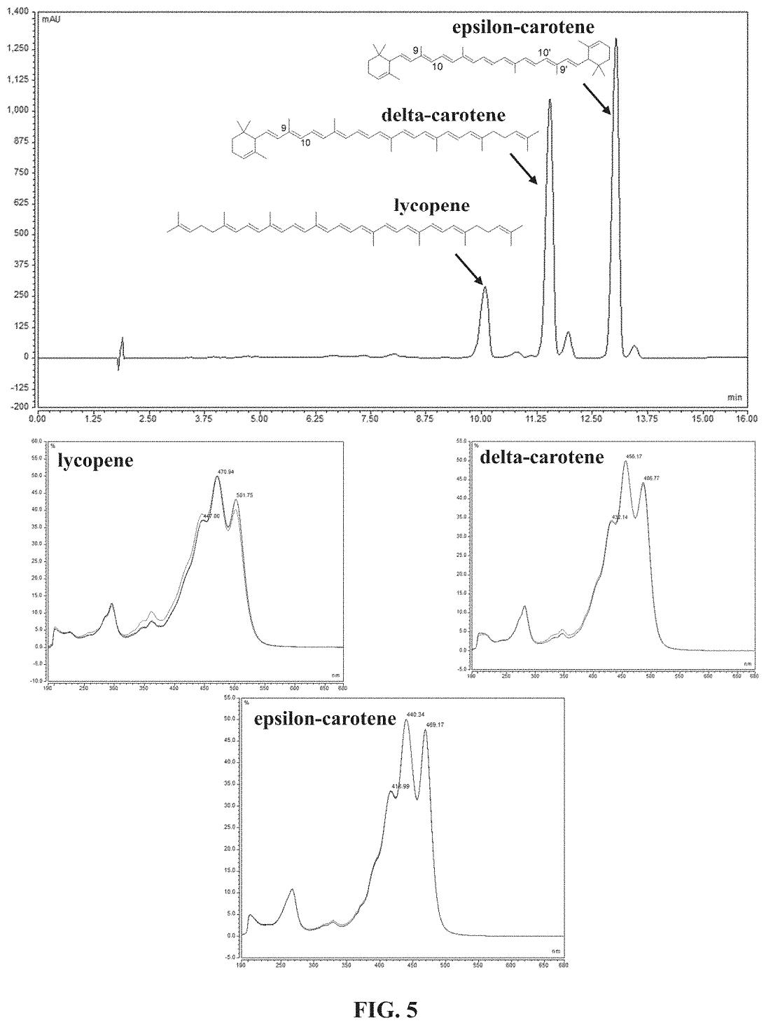

As shown in FIG. 5, the Yarrowia lipolytica strain hosting genes of HMGS, HMGR, IPI, CarRP*, CarB, FPPS:: GGPPS and EC accumulated lycopene (10 min peak), delta-carotene (11.5 min peak) and epsilon-carotene (13 min peak) by comparing the retention time (top) and the UV spectrum (bottom) of the extracted products against those of known standards.

### Example 3: Generation of Beta-Carotene Producing Yarrowia lipolytica Strains

Two genes for beta-carotene production, specifically genes encoding CarRP and CarB, and a gene encoding a fusion protein comprising FPPS fused in-frame to GGPPS, along with their own TEF promoter and XPR2 terminator, were cloned into pYconVec-Leu vector to generate a pYconVec-Leu-TEF-carB-TEF-carRP-TEF-GGPPS::FPPS construct. The pYconVec-Leu-TEF-carB-TEF-carRP-TEF-GGPPS::FPPS construct was then used to transform a Yarrowia lipolytica ATCC 90811 strain hosting pYlex-SDH-TEF-IPI-TEF-HMGR-TEF-HMGS and select on minimal media plate without leucine. The orange-colored clones were Yarrowia lipolytica beta-carotene producers. The colony with the highest beta-carotene content was named as BC-3 and chosen as the host for beta-ionone production.

### Example 4: Extraction of Beta-Carotene for HPLC Analysis

The beta-carotene producing Yarrowia lipolytica strain was streaked onto a YPD agar plate and grown at 30° C. After 2 days of incubation, the orange-colored colonies were grown in 5 ml of YPD liquid medium at 250 rpm and at 30° C. for 4 days. 0.2 ml of cell culture was harvested by centrifugation and the cell pellet was resuspended with 0.8 ml of methanol and 0.2 ml of dichloromethane. 0.5 mm diameter glass beads were then added. The Yarrowia lipolytica cells were disrupted for 1 minute in a bead beater homogenizer. Then the mixture was centrifuged for 10 min at 15,000 rpm and the supernatant was collected for HPLC analysis.

HPLC analysis of beta-carotene was performed using an Ultimate 3000 HPLC System (Dionex, Sunnyvale, Calif.) that included a quaternary pump, a temperature-controlled column compartment, an auto sampler and a UV absorbance detector. Gemini C18 (150 mm×4.6 mm 3 μm) with guard column was used for the characterization of beta-carotene. A flow rate of 1.0 ml/min was applied, and the mobile phase was composed of isopropanol (A) and acetonitrile (B). The program is 0-5 min 85% B; 5-13 min 85%-60% B; 13-14 min 60%-85% B; 14-16 min 85% B. The detection wavelengths were 474 nm for beta-carotene.

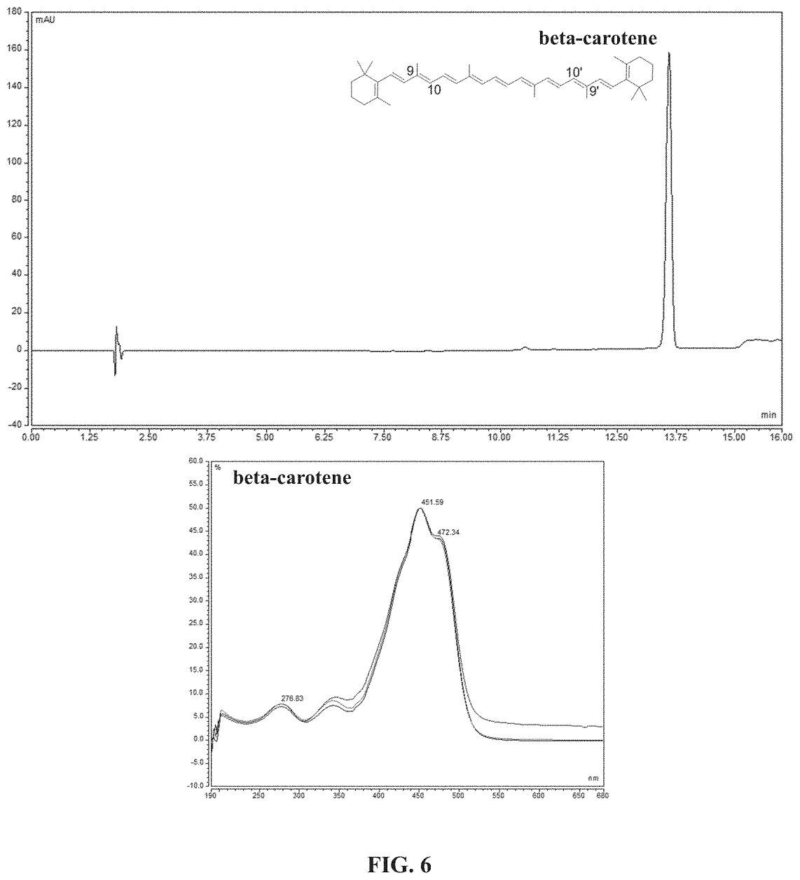

As shown in FIG. 6, the Yarrowia lipolytica hosting genes of HMGS, HMGR, IPI, carRP, carB and FPPS:: GGPPS accumulates beta-carotene (13.5 min peak) by comparing the retention time (top) and the UV spectrum (bottom) of the extracted beta-carotene against those of the beta-carotene standard.

### Example 5: Identifying Candidate CCD Enzymes with 9,10 (9′, 10′) Carotenoid Cleavage Activity

Carotenoid cleavage dioxygenase (CCD) enzymes can catalyze the cleavage of a variety of carotene substrates (including epsilon-carotene shown in FIG. 5 and beta-carotene shown in FIG. 6) to generate various ionone compounds (including alpha-ionone and beta-ionone). Referring to FIG. 2, an efficient CCD is the determining factor and key bottleneck in the biosynthetic pathway to alpha-ionone or beta-ionone.

It has been reported that some plants or cyanobacteria can make ionone compounds. Based on published literature and sequence similarity network (SSN) analysis of the known CCD gene sequences, 28 candidate CCD genes were selected for in vivo screening of the most efficient CCD enzymes in Yarrowia lipolytica. Candidate CCD genes have been individually cloned into pUC57-Kan-TEF-XPR2 vector. Each candidate CCD gene, along with its own TEF promoter and XPR2 terminator, were cloned into pYconVec-Ura-TEF-EC vector to generate pYconVec-Ura-TEF-EC-TEF-CCD constructs. Each pYconVec-Ura-TEF-EC-TEF-CCD construct was then used to transform lycopene-producing Yarrowia lipolytica ATCC 90811 host LYC-40 and select on minimal media plate without uracil. Thus, the orange-colored or yellow-colored alpha-ionone producing Yarrowia lipolytica strains were generated. Among all of the CCD enzymes that were screened, the inventors identified PhCCD1 (originated from the plant Petunia hybrida) as the CCD enzyme with the highest activity, although its alpha-ionone titer from test-tube based cell cultures was still very low (<3 mg/L). Three different versions of codon-optimized PhCCD1 gene (including SEQ ID NO: 12 and SEQ ID NO: 13) were then screened using the same strategy to identify the most efficient PhCCD1 gene in Yarrowia lipolytica ATCC 90811 (SEQ ID NO: 13).

### Example 6: Extraction of Alpha-Ionone for GC-MS Analysis

The putative orange-colored or yellow-colored alpha-ionone producing Yarrowia lipolytica clones were streaked onto minimal media plate without uracil and grown at 30° C. After 3 days of incubation, the colonies were grown in 5 ml of YPD liquid medium at 250 rpm and at 30° C. for 5 days. To analyze alpha-ionone production, 1.0 ml of the Yarrowia lipolytica cell culture was taken and the yeast slurry was extracted with 1.0 ml of n-hexane with at least 1-minute vortex for thorough mixing. After centrifugation at 2,000 rpm for 10 min, the n-hexane phase was used for GC/MS analysis.

GC/MS analysis was conducted on Shimadzu GC-2010 system coupled with GCMS-QP2010S detector. The analytical column is SHRXI-5MS (thickness 0.25 μm; length 30 m; diameter 0.25 mm) and the injection temperature is 265° C. under split mode. The temperature gradient is 0-1 min 110° C.; 1-8.75 min 110° C. to 265° C., a gradient of 20; 8.75-9.75 min, 265° C.

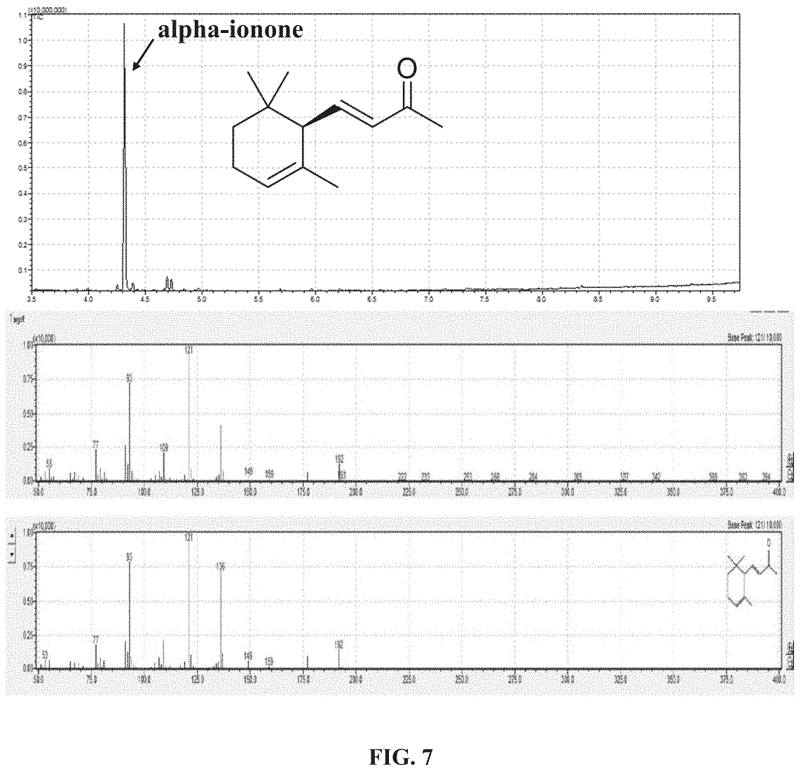

As shown in FIG. 7, the Yarrowia lipolytica strain hosting genes of HMGS, HMGR, IPI, CarRP*, CarB, FPPS:: GGPPS, EC and CCD was shown to accumulate alpha-ionone (4.3 min peak) when comparing the retention time and the mass spectrometry data of the extracted product (top, center) against the authentic alpha-ionone standard (bottom).

### Example 7: Identifying Candidate Lipid Body Compartmentalization Signal Tags to Improve PhCCD1 9,10 (9′, 10′) Carotenoid Cleavage Activity

Referring to FIG. 2, the last enzyme in the biosynthetic pathway to alpha-ionone and beta-ionone, i.e., CCD, is the key bottleneck for ionone production due to its poor activity. Efforts have been made to improve CCD carotenoid cleavage activity using E. coli as carotenoids platform. It was reported that multiple strategies including adding soluble tags to CCD, using site-directed mutagenesis, and creating carotene cyclase-CCD fusion enzymes could improve the catalytic properties of OfCCD1 in E. coli. It also has been reported that combination of site-directed mutagenesis and expressing peptide-CCD could slightly improve the PhCCD1 activity using a Saccharomyces cerevisiae platform. However, to the inventors' knowledge, no success has been achieved to improve CCD activity using the Yarrowia lipolytica platform. Besides protein engineering efforts on CCD enzymes, the inventors have chosen to investigate lipid body based subcellular compartment engineering approach to improve the cleavage activity of CCDs such as PhCCD1.

Compartmentalization of biosynthetic pathways or enzymes is an efficient strategy to increase chemical production. Pathway or enzyme compartmentalization aids chemical production by placing enzymes closer to substrates or essential co-factors, increasing the effective concentration of substrates, or providing a more suitable chemical environment for enzymatic reaction. Referring to FIG. 3, the inventors observed under microscope that red-colored lycopene and orange-colored carotene accumulated in lipid bodies in Yarrowia lipolytica cells and lipid droplets in Corynebacterium glutamicum cells. The inventors, therefore, decided to perform in vivo screening of a number of putative lipid body compartmentalization signal tags (including various lipid synthesis enzymes, membrane proteins for lipid body assembly, and lipid body structural proteins, as well as orange carotenoid binding proteins) using the Yarrowia lipolytica lycopene-producing strain LYC-40 as host.

A pYconVec-Ura-TEF-EC-TEF-PhCCD1 vector was created with a BsaI site in between the TEF promoter and the PhCCD1 gene that has been codon-optimized. Candidate genes encoding putative lipid body compartmentalization signal tags (LBT) have been individually cloned into the BsaI site to generate pYconVec-Ura-TEF-EC-TEF-LBT_CCD constructs (FIG. 4C). Each construct was then used to transform lycopene-producing Yarrowia lipolytica ATCC 90811 host LYC-40 and select on minimal media plate without uracil. Thus, the yellow-colored putative alpha-ionone producing Yarrowia lipolytica strains were generated for GC/MS screening of the most efficient lipid body compartmentalization signal tags.

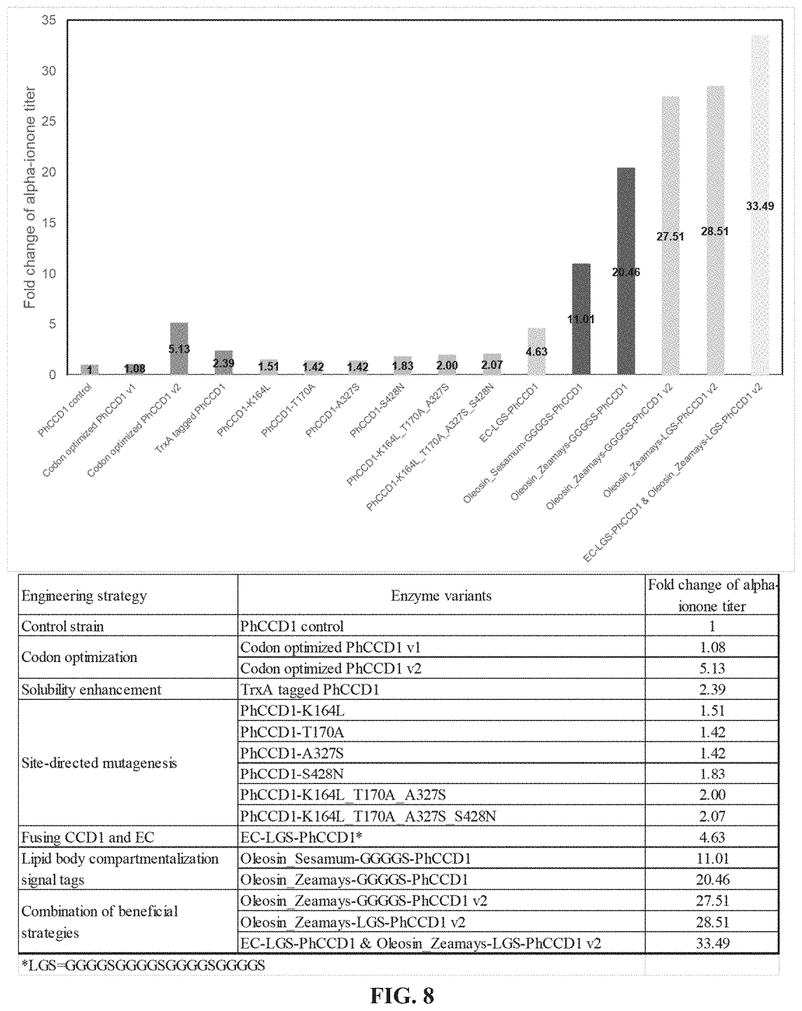

As shown in FIG. 8, comparing with strategies of adding soluble tags (e.g., TrxA tagged PhCCD1, the amino acid sequence and codon-optimized nucleic acid sequence for TrxA are provided as SEQ ID NO. 27 and SEQ ID NO. 28), site-directed mutagenesis (PhCCD1-K164L, PhCCD1-T170A, PhCCD1-A327S, PhCCD1-S428N, PhCCD1-K164L_T170A_A327S, PhCCD1-K164L_T170A_A327S_S428N, which correspond to mutants of SEQ ID NO: 11), and creating epsilon cyclase (EC)-CCD fusion enzyme (EC-LGS-PhCCD1), a number of lipid body compartmentalization signal tags could dramatically improve the in vivo carotenoids cleavage efficiency of PhCCD1 in Yarrowia lipolytica cells. These lipid body compartmentalization signal tags include Limnospira maxima orange carotenoid binding protein, Yarrowia lipolytica membrane transporter Stl1p protein, Yarrowia lipolytica lipid droplet protein Oil1p, Zea mays 16 kDa oleosin and Sesamum indicum 17 kDa oleosin. With continued reference to FIG. 8, it was shown that instead of being limited to a ˜5× increase in titer via the alternative strategies described, the present methods which make use of a fusion enzyme including a lipid body compartmentalization signal tag coupled to the CCD enzyme, the increase in titer ranges from 11× to over 33×.

### Example 8: Generation of Beta-Ionone Producing Yarrowia lipolytica Strain

Gene encoding lipid body compartmentalization tagged PhCCD1, along with TEF promoter and XPR2 terminator, was cloned into pYconVec-Ura vector to generate pYconVec-Ura-TEF-LBT_PhCCD1 constructs. The pYconVec-Ura-TEF-LBT_PhCCD1 construct was then used to transform beta-carotene producing Yarrowia lipolytica ATCC 90811 host BC-3 and select on minimal media plate without uracil. Thus, the yellow-colored beta-ionone producing Yarrowia lipolytica strains were generated. The yellow-colored clones were streaked onto minimal media plate without uracil and grown at 30° C. After 3 days incubation, the colonies were grown in 5 ml YPD liquid medium at 250 rpm and at 30° C. for 5 days. To analyze beta-ionone production, 1.0 ml of the Yarrowia lipolytica cell culture was taken and the yeast slurry was extracted with 1.0 ml of n-hexane with at least 1-minute vortex for thorough mixing. After centrifugation at 2,000 rpm for 10 min, the n-hexane phase was used for GC/MS analysis.

GC/MS analysis was conducted on Shimadzu GC-2010 system coupled with GCMS-QP2010S detector. The analytical column is SHRXI-5MS (thickness 0.25 μm; length 30 m; diameter 0.25 mm) and the injection temperature is 265° C. under split mode. The temperature gradient is 0-1 min 110° C.; 1-8.75 min 110° C. to 265° C., a gradient of 20; 8.75-9.75 min, 265° C.

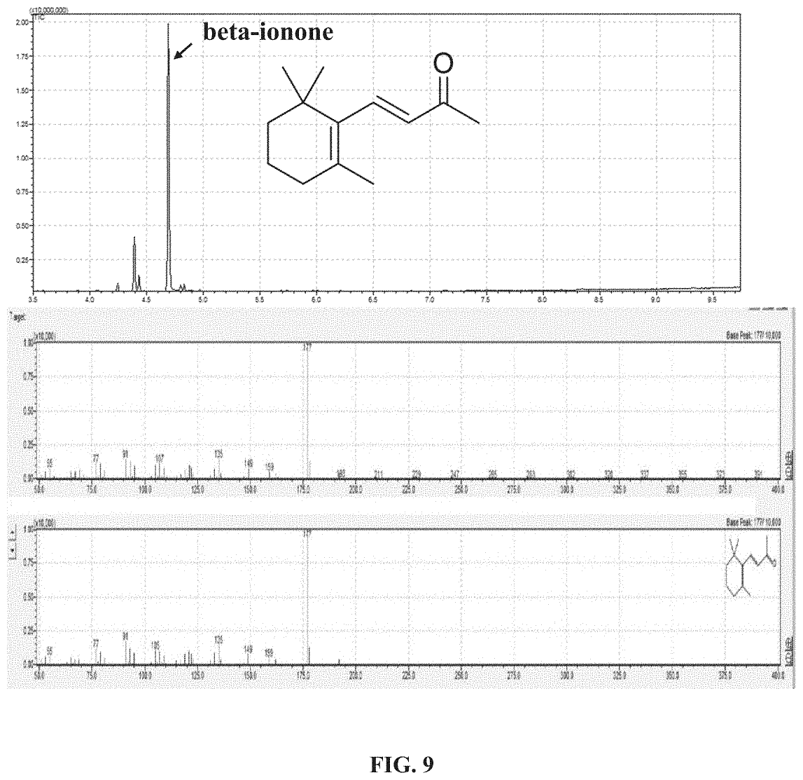

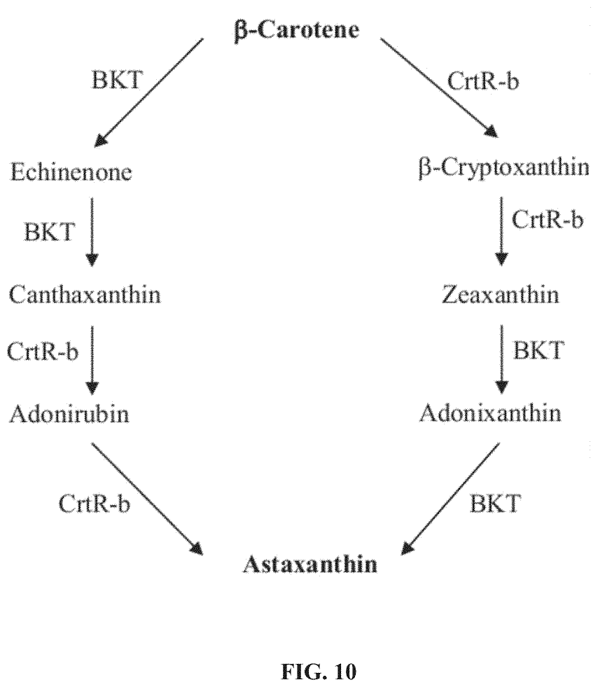

As shown in FIG. 9, the Yarrowia lipolytica strain hosting genes of HMGS, HMGR, IPI, CarRP, CarB, FPPS:: GGPPS and lipid body compartmentalization tagged PhCCD1 accumulated beta-ionone (4.7 min peak) by comparing the retention time and mass spectrometry data of the extracted product (top, center) against those of authentic beta-ionone standard (bottom).

### Example 9: Increased Production of Astaxanthin by Expressing Fusion Proteins Oleosin::crtRB and Oleosin::BKT

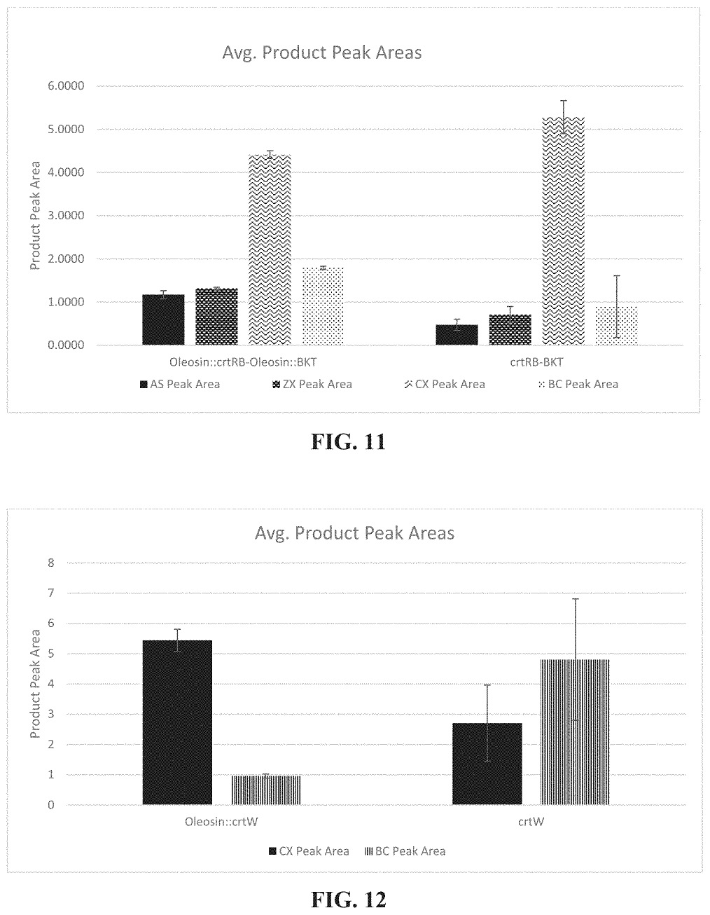

The LBT peptide described herein can be fused to other carotenoid-generating enzyme to increase production of other carotenoid compounds. FIG. 11 shows increased production of astaxanthin (AS) and zeaxanthin (ZX) when using a recombinant microbial production strain that has been transformed with a nucleic acid construct including a coding sequence encoding an LBT-CrtRB fusion enzyme and a coding sequence encoding an LBT-BKT fusion enzyme (Oleosin::crtRB-Oleosin::BKT), compared to a strain that has been transformed with a nucleic acid construct including a coding sequence encoding an untagged CrtRB enzyme and a coding sequence encoding an untagged BKT enzyme (crtRB-BKT).

### Example 10: Increased Production of Canthanxanthin by Expressing Fusion Protein Oleosin::Crt

FIG. 12 shows increased production of canthaxanthin (CX) when using a recombinant microbial production strain that has been transformed with a nucleic acid construct including a coding sequence encoding an LBT-CrtW fusion enzyme (Oleosin::crtW), compared to a strain that has been transformed with a nucleic acid construct including a coding sequence encoding an untagged CrtW enzyme (crtW).

SEQUENCES OF INTEREST
Zea mays oleosin 16 kDa
Amino acid sequence of the protein NP_001336922.1 (SEQ ID NO: 1):
MATAHHADDHRAGRRSEAVGENYMRGLYGDDDYNATHYGHQQQQRPPPAPMAVAK
ALATATAAFSMLLLSGLAVTGTVLALIVATPLMVIFSPVLVPAAITVALLTVGIVSSGGFG
VAAVAVLAWVYRYLQTTTSSSGQQPHIVKDWAQQHRLEQTRAH
Codon-optimized nucleotide sequence of NP_001336922.1 for
Yarrowia lipolytica (SEQ ID NO: 2):
ATGGCCACGGCACACCATGCTGATGATCACAGAGCTGGTCGTCGATCTGAGGCCGT
CGGTGAAAATTACATGAGAGGCCTCTACGGAGACGACGACTACAATGCAACCCATT
ACGGCCACCAACAACAACAAAGACCCCCTCCTGCCCCTATGGCTGTTGCAAAAGCT
CTTGCAACTGCAACCGCCGCTTTCTCGATGCTCCTCCTTAGCGGTCTGGCCGTTACGG
GAACCGTGCTCGCACTGATTGTTGCCACGCCTCTCATGGTCATCTTCTCCCCCGTCCT
TGTGCCCGCCGCAATCACCGTGGCCCTCCTGACAGTCGGCATTGTCTCGTCTGGCGG
TTTTGGTGTCGCTGCTGTTGCAGTTCTCGCTTGGGTGTATCGTTACCTCCAAACGACA
ACTAGCAGCTCGGGTCAGCAGCCTCACATTGTTAAAGATTGGGCACAACAGCACCG
TCTTGAGCAAACCCGTGCTCAC
Sesamum indicum oleosin 17 kDa
Amino acid sequence of the protein (SEQ ID NO: 3):
MADRDRPHPHQIQVHPQHPHRYEGGVKSLLPQKGPSTTQILAIITLLPISGTLLCLAGITL
VGTLIGLAVATPVFVIFSPVLVPAAILIAGAVTAFLTSGAFGLTGLSSLSWVLNSFRRATG
QGPLEYAKRGVQEGTLYVGEKTKQAGEAIKSTAKEGGREGTART
Codon-optimized nucleotide sequence of AAG23840.1 for
Yarrowia lipolytica (SEQ ID NO: 4):
ATGGCCGACCGTGATAGACCTCACCCTCACCAAATTCAAGTGCATCCTCAACACCCC
CATAGATACGAGGGTGGTGTTAAGTCCCTCCTCCCTCAGAAAGGCCCCTCGACCACC
CAGATTCTCGCTATCATTACCCTTCTCCCTATTAGCGGAACACTGCTCTGCCTGGCCG
GAATCACGCTGGTTGGAACCCTCATCGGCCTCGCCGTCGCAACACCTGTCTTCGTTA
TTTTCTCTCCTGTGCTCGTCCCCGCAGCCATCCTGATCGCCGGTGCCGTTACAGCATT
TCTCACTTCTGGCGCATTTGGTCTTACCGGCCTCAGCTCTCTCTCCTGGGTGCTCAAC
TCGTTCAGACGAGCTACGGGACAGGGACCCCTTGAATACGCTAAGCGAGGTGTTCA
GGAGGGTACGCTCTACGTTGGTGAGAAAACGAAGCAAGCAGGCGAGGCAATCAAGT
CCACGGCCAAAGAAGGAGGTCGAGAGGGCACTGCCCGAACA
Limnospira maxima orange carotenoid binding protein
Amino acid sequence of the protein P83689.1 (SEQ ID NO: 5):
MPFTIDTARSIFPETLAADVVPATIARFKQLSAEDQLALIWFAYLEMGKTITIAAPGAAN
MQFAENTLQEIRQMTPLQQTQAMCDLANRTDTPICRTYASWSPNIKLGFWYELGRFMD
QGLVAPIPEGYKLSANANAILVTIQGIDPGQQITVLRNCVVDMGFDTSKLGSYQRVAEPV
VPPQEMSQRTKVQIEGVTNSTVLQYMDNLNANDFDNLISLFAEDGALQPPFQKPIVGKE
NTLRFFREECQNLKLIPERGVSEPTEDGYTQIKVTGKVQTPWFGGNVGMNIAWRFLLNP
ENKVFFVAIDLLASPKELLNL
Codon-optimized nucleotide sequence of P83689.1 for
Yarrowia lipolytica (SEQ ID NO: 6):
ATGCCTTTCACTATCGATACGGCTAGAAGCATCTTCCCTGAGACACTGGCAGCCGAC
GTTGTCCCCGCCACAATCGCCCGATTTAAGCAGCTCAGCGCAGAAGACCAGCTTGCC
CTTATCTGGTTCGCTTACCTCGAGATGGGAAAGACTATCACTATCGCAGCACCCGGA
GCTGCCAATATGCAGTTCGCCGAGAACACACTTCAAGAGATCCGGCAGATGACTCC
TCTCCAGCAGACCCAGGCCATGTGTGATCTGGCTAATCGTACCGATACTCCTATTTG
TAGAACTTACGCTTCTTGGTCCCCCAATATTAAACTTGGTTTCTGGTATGAACTCGGC
CGGTTTATGGACCAGGGTCTTGTGGCCCCCATTCCCGAAGGTTACAAGCTCTCTGCT
AATGCAAATGCAATTCTTGTGACCATCCAAGGAATTGATCCTGGACAGCAAATCACG
GTCCTGCGGAATTGCGTTGTTGATATGGGTTTCGACACATCTAAGCTCGGTTCGTAC
CAGCGGGTTGCTGAGCCCGTCGTTCCCCCCCAGGAGATGTCCCAACGAACTAAGGTT
CAAATTGAGGGAGTTACTAACTCTACCGTCCTTCAGTACATGGATAATCTGAACGCA
AACGATTTTGATAATCTTATCTCGCTTTTCGCCGAGGATGGTGCTCTTCAGCCCCCTT
TTCAAAAGCCTATCGTTGGCAAAGAGAATACACTCAGATTTTTCCGAGAGGAATGCC
AGAACCTCAAACTCATCCCTGAGCGTGGAGTTTCGGAACCCACAGAGGATGGATAC
ACACAGATTAAGGTCACGGGCAAAGTTCAGACACCCTGGTTCGGCGGAAATGTGGG
AATGAACATCGCTTGGCGTTTCCTTCTTAACCCTGAGAACAAGGTTTTCTTTGTTGCA
ATCGATCTGCTCGCAAGCCCCAAAGAGCTCCTCAACCTT
Yarrowia lipolytica membrane transporter Stl1p
Amino acid sequence of the protein XP_501907.1 (SEQ ID NO: 7):
MKLQVPAFARSSSDVKTSFMGARGQKLHNLVAAIAGLGFLLFGYDQGVMGGLLTLDTF
IQQFPKMDTSDYLPPKVKTFNTTIQGTAVGIYEIGCMIGALFTMWAGDKLGRRYMIFFGS
IIMTIGAILQCASYSLGQFIAGRVISGIGNGFITATVPMLQSECAKPERRGKLVMLEGALIT
AGIALSYWIDFGFYWVRTNDADWRFPIAFQIVFSLVLTFTIMSLPESPRWLVKKQRFEEA
AGVFAALEDVPLDDPYVINQITSVKESIMMEQLAQLGVDGVDARRKIQSGEFQMGQELS
FIGQMKLMFTFGKKKNFHRTMLAYWNQVMQQVTGINLITYYAAYIYQTSVGMNATDS
RILAACNGTEYFMASWVAFYTIERFGRRKLMLFGAVGQACTMAILTGCVYAASKPEDG
GLDMQGAGIAAAVFLFVFNTFFAIGWLGMTWLYPAEISSLEIRAPANGLSTSGNWAFNF
MVVMITPVAFNSIKWKTYIIFACINAFMVPMVYFFYPETAGRSLEEIDMIFAESNPRTPW
DVVGIANRLPKNSVATYDDYEAGEQEEKAIVETAESVSHDSAEFQ
Codon-optimized nucleotide sequence of XP_501907.1 for
Yarrowia lipolytica (SEQ ID NO: 8):
ATGAAACTGCAAGTGCCCGCTTTCGCTCGGTCCTCCTCCGACGTGAAGACCTCTTTC
ATGGGTGCACGTGGACAAAAACTTCATAACCTTGTTGCCGCAATCGCCGGTCTTGGA
TTTCTTCTGTTCGGATACGACCAAGGCGTCATGGGTGGCCTGCTTACGCTCGATACG
TTCATCCAACAGTTTCCCAAAATGGATACCTCCGATTATCTGCCCCCTAAGGTTAAG
ACGTTTAACACGACAATTCAGGGAACAGCTGTGGGAATTTACGAAATTGGTTGTATG
ATTGGCGCTCTCTTCACAATGTGGGCTGGTGATAAGCTGGGCCGACGATACATGATC
TTCTTTGGCTCTATTATCATGACCATCGGTGCCATTCTTCAGTGTGCCAGCTATTCCC
TGGGCCAATTCATTGCTGGAAGAGTGATTTCTGGTATCGGAAACGGTTTCATCACGG
CTACTGTCCCTATGCTTCAGTCGGAATGCGCCAAGCCCGAAAGACGTGGCAAACTG
GTGATGCTGGAAGGAGCTCTGATCACGGCAGGAATTGCACTCAGCTACTGGATTGAT
TTCGGTTTTTATTGGGTCCGAACTAACGATGCAGATTGGCGGTTTCCTATCGCTTTTC
AGATCGTGTTTTCTCTTGTTCTGACTTTTACTATTATGTCCCTTCCCGAGAGCCCCAG
ATGGCTCGTCAAAAAGCAGCGATTCGAGGAGGCTGCAGGTGTCTTCGCTGCACTTGA
GGATGTTCCCCTTGATGATCCCTACGTTATCAACCAGATCACATCCGTCAAAGAATC
TATCATGATGGAACAGCTGGCCCAACTTGGTGTTGACGGTGTTGATGCTAGAAGAAA
AATTCAGAGCGGAGAGTTCCAAATGGGTCAGGAGCTTTCTTTTATCGGCCAGATGAA
GCTGATGTTCACCTTCGGTAAGAAAAAAAATTTTCACCGTACAATGCTGGCCTATTG
GAATCAAGTGATGCAACAGGTGACAGGTATTAACCTCATCACTTACTATGCCGCCTA
CATTTACCAGACCAGCGTTGGAATGAATGCCACCGACTCCCGTATTCTCGCTGCCTG
CAATGGTACGGAATATTTCATGGCTTCCTGGGTTGCCTTCTACACGATTGAACGGTT
CGGTCGTCGGAAACTTATGCTGTTCGGCGCTGTGGGCCAAGCCTGCACTATGGCTAT
CCTCACGGGCTGCGTTTATGCAGCCTCCAAGCCCGAAGATGGAGGCCTCGACATGC
AAGGCGCCGGTATTGCTGCAGCTGTTTTCCTTTTCGTTTTCAACACATTCTTCGCCAT
CGGATGGCTTGGTATGACCTGGCTCTATCCCGCTGAAATTTCCTCGCTCGAGATTCG
AGCACCTGCTAATGGACTCAGCACTAGCGGTAACTGGGCTTTCAATTTCATGGTCGT
TATGATTACGCCCGTTGCATTTAATTCTATTAAATGGAAGACCTACATTATTTTTGCA
TGCATCAACGCATTTATGGTGCCTATGGTGTATTTCTTCTACCCCGAAACTGCAGGTC
GGTCCCTGGAGGAAATTGACATGATTTTCGCCGAATCCAATCCCCGAACACCCTGGG
ATGTCGTCGGCATTGCTAACCGGCTGCCCAAGAATAGCGTGGCCACGTATGATGATT
ACGAGGCTGGCGAGCAGGAAGAAAAAGCCATCGTCGAAACTGCAGAGTCTGTCTCC
CACGATTCCGCCGAGTTTCAA
Yarrowia lipolytica lipid droplet protein Oil1p
Amino acid sequence of the protein XP_505819.1 (SEQ ID NO: 9):
MPIKDFTNPAFSNPQETSSQHTHTKMPSVADNTSNGPIEGNVLPQSKFIQHLSEYPAVAA
VTGFAASFPVVKIFASNAVPLIQAIQNRGAPVAEPVVKRAAPYISQIDNAADEALNRLDK
AVPSLKNTKPDEVYSRIVTQPLENVRGTVDKYADETKNTVSRVVVQPIRDVASRVQSQV
VTYYDAHGKPIVHARLDPIFHPLNDRLEALINAYLPKGQEIVTDAENELARAWRLTVVA
FDRARPLIEQQTSQIQEINQHTREHIQKVYDGKRSEIDDKKTVSGPVYATVATVRDLSQE
GLQYAQSILNAKKPEEKADSNSGVAPVSQPHTTSAVDNSLAAPTAVHEVTASA
Codon-optimized nucleotide sequence of XP_505819.1 for
Yarrowia lipolytica (SEQ ID NO: 10):
ATGCCTATTAAGGATTTTACCAATCCTGCATTTTCCAATCCCCAAGAAACATCCTCTC
AGCACACACATACAAAGATGCCTTCGGTCGCTGATAATACCTCCAACGGCCCCATTG
AGGGAAACGTGCTGCCTCAATCCAAGTTCATTCAGCATCTCTCTGAGTACCCTGCAG
TTGCAGCAGTCACAGGTTTCGCCGCATCCTTCCCTGTTGTCAAAATTTTTGCCAGCAA
TGCTGTTCCCCTTATCCAAGCCATTCAGAACCGTGGAGCTCCCGTGGCAGAACCTGT
GGTCAAACGTGCAGCCCCTTATATCTCCCAGATTGATAACGCTGCCGACGAGGCTCT
GAATCGGCTGGATAAGGCAGTCCCTTCTCTCAAAAATACCAAACCCGATGAAGTTTA
TTCCAGAATTGTGACCCAACCCCTGGAAAACGTGCGTGGCACTGTGGACAAATATGC
AGATGAGACGAAAAACACAGTCTCCCGGGTTGTTGTTCAGCCTATCAGAGATGTTGC
CTCTAGAGTTCAGTCTCAGGTTGTTACATATTATGATGCTCATGGAAAACCCATCGT
GCATGCACGGCTCGATCCCATTTTCCACCCCCTTAATGATAGACTTGAAGCTCTGATT
AATGCTTACCTGCCTAAGGGACAGGAGATCGTGACGGATGCCGAGAACGAGCTCGC
ACGAGCCTGGCGACTTACAGTTGTTGCATTCGATCGAGCCCGTCCTCTTATTGAGCA
ACAAACCTCGCAGATTCAAGAGATCAATCAACACACTCGGGAGCATATTCAAAAGG
TCTACGACGGCAAACGATCCGAGATCGACGACAAGAAGACTGTTTCTGGACCTGTG
TATGCAACCGTGGCCACGGTTCGGGATCTGTCCCAAGAAGGACTTCAATATGCTCAA
TCTATTCTGAACGCAAAGAAGCCCGAAGAGAAAGCCGACAGCAACAGCGGCGTCGC
ACCCGTCTCGCAACCCCACACCACGTCCGCTGTGGATAATAGCCTTGCTGCCCCTAC
TGCTGTGCATGAAGTCACTGCCAGCGCA
Petunia x hvbrida carotenoid cleavage dioxygenase 1
Amino acid sequence of the protein AAT68189.1 (SEQ ID NO: 11):
MGRKESDDGVERIEGGVVVVNPKPKKGITAKAIDLLEKVIIKLMHDSSKPLHYLSGNFA
PTDETPPLNDLPIKGHLPECLNGEFVRVGPNPKFAPVAGYHWFDGDGMIHGLRIKDGKA
TYVSRYVRTSRLKQEEFFEGAKFMKIGDLKGLFGLFTVYMQMLRAKLKILDTSYGNGT
ANTALVYHHGKLLALSEADKPYALKVLEDGDLQTLGMLDYDKRLLHSFTAHPKVDPV
TGEMFTFGYAHEPPYITYRVISKDGIMQDPVPITIPEAIMMHDFAITENYAIMMDLPLCFR
PKEMVKNNQLAFTFDTTKKARFGVLPRYAKSEALIRWFELPNCFIFHNANAWEEGDEV
VLITCRLPHPDLDMVNGEVKENLENFSNELYEMRFNMKSGAASQKKLSESSVDFPRINE
NYTGRKQRYVYGTTLNSIAKVTGIIKFDLHAEPETGKKQLEVGGNVQGIFDLGPGRFGSE
AVFVPSQPGTECEEDDGYLIFFVHDENTGKSAVNVIDAKTMSAEPVAVVELPKRVPYGF
HAFFVTEEQIQEQAKL
Codon-optimized nucleotide sequence of AAT68189.1 for
Yarrowia lipolytica version 1 (SEQ ID NO: 12):
ATGGGGCGAAAGGAGTCGGACGACGGCGTTGAGCGAATCGAGGGCGGCGTGGTGG
TTGTTAACCCCAAGCCCAAGAAGGGCATCACCGCCAAGGCAATTGATCTCCTGGAG
AAGGTTATAATTAAGCTTATGCACGACTCCTCCAAGCCCCTGCATTACCTGTCTGGG
AACTTCGCTCCCACCGACGAGACTCCACCCCTGAACGATCTGCCCATAAAGGGGCA
CCTGCCCGAGTGTCTGAACGGAGAGTTCGTGCGAGTGGGACCGAACCCCAAGTTCG
CCCCCGTGGCCGGCTATCATTGGTTCGACGGCGACGGGATGATCCATGGGCTGCGCA
TCAAGGACGGCAAGGCTACGTACGTATCCAGATACGTTCGAACCAGTAGACTTAAG
CAGGAGGAGTTTTTCGAGGGCGCCAAGTTCATGAAGATCGGCGACCTGAAGGGGCT
TTTCGGACTTTTTACCGTGTACATGCAGATGCTTCGAGCTAAGCTGAAGATCCTGGA
TACAAGCTACGGCAACGGGACCGCCAACACCGCCCTTGTTTACCATCACGGGAAGC
TGCTGGCTCTGTCTGAGGCCGACAAGCCCTACGCTCTGAAGGTGCTTGAGGACGGCG
ACCTCCAGACCCTGGGGATGCTTGACTACGATAAGCGACTGCTGCACTCTTTTACCG
CACACCCCAAGGTGGATCCCGTTACCGGAGAGATGTTCACATTCGGGTATGCCCATG
AGCCGCCCTATATAACCTACCGAGTGATTTCCAAGGACGGCATCATGCAGGACCCG
GTACCCATTACCATTCCCGAGGCCATAATGATGCACGACTTCGCCATCACCGAGAAC
TACGCTATCATGATGGACCTTCCCCTGTGCTTTCGCCCCAAGGAGATGGTTAAGAAC
AATCAGCTTGCCTTCACATTTGACACCACCAAGAAGGCCCGGTTCGGCGTACTGCCC
CGGTACGCTAAGTCGGAGGCACTTATACGATGGTTTGAGCTGCCCAACTGTTTTATT
TTCCATAATGCAAACGCCTGGGAGGAGGGCGACGAGGTAGTTCTGATCACATGTCG
CCTGCCGCACCCCGATCTTGATATGGTAAACGGCGAGGTGAAGGAGAACCTGGAGA
ACTTTAGCAACGAGCTTTACGAGATGCGATTCAATATGAAGTCGGGAGCTGCCTCCC
AGAAGAAGCTGTCCGAGTCCTCTGTGGACTTCCCCCGCATAAACGAGAATTATACCG
GCCGAAAGCAGAGATACGTTTACGGCACAACCCTGAATAGCATCGCAAAGGTTACC
GGCATAATAAAGTTCGACCTGCACGCCGAGCCCGAAACCGGGAAGAAGCAGCTGGA
GGTGGGCGGCAACGTGCAGGGGATATTCGATCTTGGCCCCGGGCGATTCGGAAGTG
AGGCCGTTTTCGTGCCATCTCAGCCCGGAACCGAGTGCGAGGAGGACGACGGCTAT
CTGATTTTTTTTGTTCACGACGAGAATACCGGCAAGTCCGCCGTTAACGTTATCGAC
GCTAAGACCATGTCCGCTGAGCCCGTTGCTGTGGTGGAGCTTCCAAAGCGAGTGCCC
TACGGCTTTCACGCATTTTTCGTAACCGAGGAGCAGATCCAGGAGCAGGCAAAGCTT
TAA
Codon-optimized nucleotide sequence of AAT68189.1 for
Yarrowia lipolytica version 2 (SEQ ID NO: 13):
ATGGGCCGAAAGGAGTCTGACGACGGCGTGGAGCGAATCGAGGGCGGCGTGGTGGT
GGTGAACCCCAAGCCCAAGAAGGGCATCACCGCCAAGGCCATCGACCTGCTGGAGA
AGGTGATCATCAAGCTGATGCACGACTCTTCTAAGCCCCTGCACTACCTGTCTGGCA
ACTTCGCCCCCACCGACGAGACTCCCCCCCTGAACGACCTGCCCATCAAGGGCCACC
TGCCCGAGTGCCTGAACGGCGAGTTCGTGCGAGTGGGCCCCAACCCCAAGTTCGCC
CCCGTGGCCGGCTACCACTGGTTCGACGGCGACGGCATGATCCACGGCCTGCGAAT
CAAGGACGGCAAGGCCACCTACGTGTCTCGATACGTGCGAACCTCTCGACTGAAGC
AGGAGGAGTTCTTCGAGGGCGCCAAGTTCATGAAGATCGGCGACCTGAAGGGCCTG
TTCGGCCTGTTCACCGTGTACATGCAGATGCTGCGAGCCAAGCTGAAGATCCTGGAC
ACCTCTTACGGCAACGGCACCGCCAACACCGCCCTGGTGTACCACCACGGCAAGCT
GCTGGCCCTGTCTGAGGCCGACAAGCCCTACGCCCTGAAGGTGCTGGAGGACGGCG
ACCTCCAGACCCTGGGCATGCTGGACTACGACAAGCGACTGCTGCACTCTTTCACCG
CCCACCCCAAGGTGGACCCCGTGACCGGCGAGATGTTCACCTTCGGCTACGCCCACG
AGCCCCCCTACATCACCTACCGAGTGATCTCTAAGGACGGCATCATGCAGGACCCCG
TGCCCATCACCATCCCCGAGGCCATCATGATGCACGACTTCGCCATCACCGAGAACT
ACGCCATCATGATGGACCTGCCCCTGTGCTTCCGACCCAAGGAGATGGTGAAGAAC
AACCAGCTGGCCTTCACCTTCGACACCACCAAGAAGGCCCGATTCGGCGTGCTGCCC
CGATACGCCAAGTCTGAGGCCCTGATCCGATGGTTCGAGCTGCCCAACTGCTTCATC
TTCCACAACGCCAACGCCTGGGAGGAGGGCGACGAGGTGGTGCTGATCACCTGCCG
ACTGCCCCACCCCGACCTGGACATGGTGAACGGCGAGGTGAAGGAGAACCTGGAGA
ACTTCTCTAACGAGCTGTACGAGATGCGATTCAACATGAAGTCTGGCGCCGCCTCTC
AGAAGAAGCTGTCTGAGTCCTCTGTGGACTTCCCCCGAATCAACGAGAACTACACC
GGCCGAAAGCAGCGATACGTGTACGGCACCACCCTGAACTCTATCGCCAAGGTGAC
CGGCATCATCAAGTTCGACCTGCACGCCGAGCCCGAGACTGGCAAGAAGCAGCTGG
AGGTGGGCGGCAACGTGCAGGGCATCTTCGACCTGGGCCCCGGCCGATTCGGCTCT
GAGGCCGTGTTCGTGCCCTCTCAGCCCGGCACCGAGTGCGAGGAGGACGACGGCTA
CCTGATCTTCTTCGTGCACGACGAGAACACCGGCAAGTCTGCCGTGAACGTGATCGA
CGCCAAGACCATGTCTGCCGAGCCCGTGGCCGTGGTGGAGCTGCCCAAGCGAGTGC
CCTACGGCTTCCACGCCTTCTTCGTGACCGAGGAGCAGATCCAGGAGCAGGCCAAG
CTGTAA
Osmanthus frakrans carotenoid cleavage dioxygenase 1
Amino acid sequence of the protein BAJ05401.1 (SEQ ID NO: 14):
MGMQGEDAQRTGNIVAVKPKPSQGLTSKAIDWLEWLFVKMMHDSKQPLHYLSGNFAP
VDETPPLKDLPVTGHLPECLNGEFVRVGPNPKFASIAGYHWFDGDGMIHGMRIKDGKA
TYVSRYVQTSRLKQEEFFGRAMFMKIGDLKGMFGLLMVNMQMLRAKLKVLDISYGIG
TANTALVYHHGKLLALSEADKPYAIKVLEDGDLQTIGLLDYDKRLAHSFTAHPKVDPFT
GEMFTFGYSHTPPYVTYRVISKDGAMNDPVPITVSGPIMMHDFAITENYAIFMDLPLYFK
PKEMVKDKKFIFSFDATQKARFGILPRYAKNELLIKWFELPNCFIFHNANAWEEGDEVV
LITCRLENPDLDMVNSTVKERLDNFKNELYEMRFNLQNGLASQKKLSVSAVDFPRVNES
YTTRKQRYVYGTTLDKIAKVTGIIKFDLHAEPETGKEKLELGGNVKGIFDLGPGRFGSEA
VFVPRHPGITSEEDDGYLIFFVHDENTGKSAVNVIDAKTMSPDPVAVVELPKRVPYGFH
AFFVTEDQLQEQAKV
Osmanthus fragrans CCD1 mRNA for carotenoid cleavage
dioxygenase 1, nucleotide sequence of AB526197.1 (SEQ ID NO: 15):
ATGGGGATGCAAGGAGAGGATGCGCAGCGGACTGGGAATATTGTTGCTGTAAAGCC
GAAACCCAGTCAAGGGCTCACTTCCAAGGCCATAGACTGGTTGGAATGGCTGTTTGT
GAAAATGATGCATGACTCCAAACAGCCTCTCCATTATCTTTCTGGGAATTTTGCTCC
AGTTGATGAGACTCCTCCTCTTAAGGACCTTCCTGTTACAGGGCACCTCCCTGAGTG
TCTGAATGGTGAATTTGTGAGGGTTGGTCCTAACCCCAAGTTTGCTTCAATTGCTGGT
TATCATTGGTTTGATGGAGATGGAATGATTCATGGTATGCGGATAAAAGATGGGAA
AGCAACATATGTCTCGCGTTATGTGCAGACATCTCGCCTTAAACAAGAAGAGTTTTT
TGGCAGAGCTATGTTTATGAAGATTGGAGACCTGAAAGGGATGTTTGGATTGCTCAT
GGTTAACATGCAAATGCTCAGAGCAAAACTGAAAGTATTAGATATTTCCTATGGAAT
TGGTACAGCTAATACTGCTCTGGTATACCACCATGGAAAGCTTTTGGCACTTAGTGA
GGCAGATAAACCATATGCCATTAAAGTTTTGGAAGATGGAGATCTGCAAACTATTG
GCTTGCTGGACTATGATAAAAGACTGGCACATTCATTTACTGCTCATCCTAAGGTTG
ACCCATTTACCGGAGAGATGTTTACCTTTGGCTATTCACATACACCACCTTATGTCAC
ATACAGAGTTATATCGAAGGATGGAGCGATGAATGATCCCGTTCCAATAACAGTAT
CAGGCCCAATCATGATGCATGATTTTGCGATTACTGAAAATTATGCAATTTTTATGG
ACCTCCCTTTATACTTTAAGCCAAAGGAAATGGTGAAGGATAAAAAGTTTATTTTCA
GTTTTGATGCTACCCAGAAAGCTCGTTTTGGCATCCTTCCGCGGTATGCAAAGAATG
AGCTACTAATCAAATGGTTTGAGCTTCCAAATTGCTTCATATTCCATAATGCAAATG
CTTGGGAGGAAGGAGATGAAGTTGTTCTGATCACTTGTCGCCTTGAGAATCCAGATC
TTGACATGGTTAACAGCACCGTTAAAGAAAGGCTTGATAATTTCAAAAACGAACTGT
ACGAGATGAGGTTCAACCTCCAAAATGGTCTGGCTTCACAGAAAAAACTATCTGTAT
CCGCTGTAGATTTTCCAAGGGTGAACGAAAGTTACACTACTAGGAAACAGCGATAT
GTATATGGAACAACACTGGACAAGATTGCTAAGGTAACTGGGATTATCAAGTTTGAT
TTGCATGCGGAACCAGAGACTGGAAAAGAAAAGCTAGAACTTGGAGGAAATGTTAA
AGGTATCTTTGATCTAGGACCTGGAAGATTTGGTTCAGAGGCTGTCTTTGTTCCGCG
GCATCCTGGTATCACATCTGAAGAAGATGATGGCTACTTGATTTTCTTTGTACATGAT
GAGAACACTGGAAAGTCGGCAGTGAATGTAATTGATGCAAAAACAATGTCACCTGA
TCCTGTTGCTGTTGTCGAACTGCCCAAGAGAGTTCCGTACGGATTTCATGCCTTCTTC
GTGACAGAAGACCAACTTCAAGAACAGGCAAAGGTTTGA
Zea mays carotenoid cleavage dioxygenase 1
Amino acid sequence of the protein ACG46084.1 (SEQ ID NO: 16):
MGTEAEQPDMDSHRNDGVVVVPAPRPRKGLASWALDLLESLAVRLGHDKTKPLHWLS
GNFAPVVEETPPAPNLTVRGHLPECLNGEFVRVGPNPKFAPVAGYHWFDGDGMIHAMR
IKDGKATYVSRYVKTARLKQEEYFGGAKFMKIGDLKGFFGLFMVQMQQLRKKFKVLD
FTYGFGTANTALIYHHGKLMALSEADKPYVVKVLEDGDLQTLGLLDYDKRLKHSFTAH
PKVDPFTDEMFTFGYSHEPPYCTYRVINKEGAMLDPVPITIPESVMMHDFAITENYSIFM
DLPLLFRPKEMVKNGEFIYKFDPTKKGRFGILPRYAKDDKLIRWFQLPNCFIFHNANAW
EEGDEVVLITCRLENPDLDKVNGYQSDKLENFGNELYEMRFNMKTGAASQKQLSVSAV
DFPRVNESYTGRKQRYVYCTILDSIAKVTGIIKFDLHAEPESGVKVLEVGGNVQGIYDLG
PGRFGSEAIFVPKHPGVSGEEDDGYLIFFVHDENTGKSEVNVIDAKTMSADPVAVVELP
NRVPYGFHAFFVTEDQLARQAEGQ
Zea mays carotenoid cleavage dioxygenase 1 (CCD1)
mRNA, nucleotide sequence of DQ100346.1 (SEQ ID NO: 17):
ATGGGGACGGAGGCGGAGCAGCCGGACATGGACAGCCACCGAAACGACGGCGTCG
TGGTGGTGCCAGCGCCGCGCCCGCGTAAGGGGCTCGCTTCCTGGGCGCTCGACCTGC
TTGAGTCCCTCGCCGTGCGCCTCGGCCACGACAAGACCAAGCCGCTCCACTGGCTCT
CCGGCAACTTCGCCCCCGTCGTCGAGGAGACCCCGCCGGCCCCAAACCTTACCGTCC
GCGGACACCTCCCGGAGTGCTTGAATGGAGAGTTTGTCAGGGTTGGGCCTAATCCGA
AGTTTGCTCCTGTTGCGGGGTATCACTGGTTTGATGGAGACGGGATGATTCATGCCA
TGCGTATTAAGGATGGAAAAGCTACCTATGTATCAAGATATGTGAAGACTGCCCGCC
TCAAACAAGAGGAGTATTTTGGTGGAGCAAAGTTTATGAAGATTGGAGACCTTAAG
GGATTTTTTGGATTGTTTATGGTCCAAATGCAGCAACTTCGGAAAAAATTCAAAGTC
TTGGATTTTACCTATGGATTTGGGACAGCTAATACTGCACTTATATATCATCATGGTA
AACTCATGGCCTTGTCAGAAGCAGATAAGCCATATGTTGTTAAGGTCCTTGAAGATG
GAGACTTGCAGACTCTTGGCTTGTTGGATTATGACAAAAGGTTGAAACATTCTTTTA
CTGCCCATCCAAAGGTTGACCCTTTTACAGATGAAATGTTCACATTCGGATATTCAC
ATGAACCTCCATACTGTACATACCGTGTGATTAACAAAGAAGGAGCTATGCTTGATC
CTGTGCCAATAACAATACCGGAATCTGTAATGATGCATGATTTTGCCATCACAGAGA
ATTACTCTATTTTTATGGACCTCCCTTTATTGTTCCGACCAAAGGAAATGGTGAAGA
ACGGTGAGTTTATCTACAAGTTTGATCCTACAAAGAAAGGTCGTTTTGGTATTCTCC
CCCGCTATGCAAAGGATGACAAACTCATCAGATGGTTTCAACTCCCTAATTGTTTCA
TATTCCATAATGCTAATGCTTGGGAAGAGGGTGATGAAGTTGTTCTAATTACCTGCC
GCCTTGAGAATCCAGATTTGGACAAGGTGAATGGATATCAAAGTGACAAGCTCGAA
AACTTCGGGAATGAGCTGTACGAGATGAGATTCAACATGAAAACGGGTGCTGCTTC
ACAAAAGCAATTGTCTGTTTCTGCTGTGGATTTTCCTCGTGTTAATGAGAGCTATACT
GGCAGAAAGCAGCGGTATGTCTACTGCACTATACTTGACAGCATTGCGAAGGTGAC
TGGCATCATAAAGTTTGATCTGCATGCTGAACCGGAAAGTGGTGTGAAAGTACTTGA
AGTGGGAGGAAATGTACAAGGCATATATGACCTGGGACCTGGTAGATTTGGTTCAG
AGGCGATTTTTGTTCCCAAGCATCCAGGTGTGTCTGGAGAAGAAGATGACGGCTATT
TGATATTCTTTGTACACGACGAGAACACAGGGAAATCTGAAGTAAATGTTATCGATG
CAAAGACAATGTCTGCTGATCCAGTTGCGGTGGTTGAGCTTCCTAATAGGGTTCCTT
ATGGATTCCATGCCTTTTTTGTAACTGAGGACCAACTGGCTCGACAGGCGGAGGGGC
AGTGA
Nostocales cyanobacterium HT-58-2 9-cis-epoxycarotenoid dioxygenase
Amino acid sequence of the protein WP_087544521.1 (SEQ ID NO: 18):
MAIKQVNPFLDGNFAPVEEEITTNTLQVIGELPPDLSGMFVRNGPNPQWTPIGQYHWFD
GDGMLHGVRISNGKATYRNRYVRTKGWKIENEAGKAVWTGLLEPRPKHTPHGQSKNT
ANTALVWHAGQLLALWEGGAPHAIKVPELKTIGEYTYNGKLVSAVTAHPKVDPVTGE
MMFFGYSFAPPYLQYSVVSPQGELLQTETIDIPMAVMMHDFAITEDYTIFMDLPLTFSQE
RIKRGEPLMMFERDKPSRFGIVPRYGNNSNIRWFESPACYIFHTVNAYEEGDEVVLVAC
RMSSTTVLGAPQDTHVDSEADIPRLHRWRFNLKTGKVSEEMLDDVPSEFPRVNENLLG
QKTRYGYTGRMAKSSLPLFDGLIKYDLNNGKSQTHEFGSGRYGGEAVFVPRPGATAED
DGWLVTFVHDTAKDTSELVVVSAQDVTGEPVARVLIPQRVPYGFHGAWVSEEQLKAS
M
Codon-optimized nucleotide sequence of WP_087544521.1 for
Yarrowia lipolytica (SEQ ID NO: 19):
ATGGCAATTAAGCAGGTCAATCCCTTCCTTGACGGTAACTTCGCACCCGTTGAAGAG
GAGATCACAACCAACACGCTGCAGGTGATTGGAGAACTGCCCCCTGACCTCTCGGG
AATGTTTGTTCGAAATGGCCCTAATCCCCAGTGGACCCCTATCGGACAGTATCACTG
GTTCGACGGCGATGGCATGCTCCACGGCGTTCGTATTTCGAACGGCAAAGCTACTTA
CCGTAATCGGTACGTTCGAACAAAAGGATGGAAGATCGAAAACGAAGCTGGTAAAG
CCGTGTGGACCGGACTCCTTGAGCCCCGACCTAAACACACTCCTCATGGTCAATCGA
AGAACACAGCAAACACAGCCCTTGTGTGGCATGCAGGCCAACTGCTGGCACTCTGG
GAGGGCGGCGCCCCTCACGCAATCAAAGTTCCTGAGCTCAAGACGATCGGCGAATA
TACTTACAATGGAAAACTCGTGTCTGCTGTCACTGCCCATCCCAAAGTGGATCCCGT
CACGGGCGAGATGATGTTCTTTGGATACTCGTTCGCCCCTCCTTACCTGCAGTACTCT
GTGGTTTCCCCCCAGGGAGAACTTCTGCAAACGGAAACCATCGACATTCCTATGGCA
GTGATGATGCACGACTTTGCTATTACTGAAGATTATACTATTTTCATGGACCTTCCCC
TTACATTTTCTCAGGAGAGAATCAAGCGAGGAGAGCCCCTTATGATGTTTGAAAGAG
ATAAGCCTAGCCGATTCGGTATTGTGCCTCGTTATGGAAATAACTCTAACATTAGAT
GGTTCGAATCGCCCGCATGCTATATCTTCCACACGGTTAATGCATACGAAGAAGGAG
ATGAGGTTGTCCTGGTGGCCTGTCGAATGTCCTCTACGACTGTCCTGGGAGCACCTC
AAGACACACATGTCGATTCTGAAGCCGATATTCCCAGACTCCATCGATGGCGGTTCA
ATCTGAAGACTGGTAAGGTGTCGGAGGAAATGCTTGACGACGTCCCCTCGGAATTTC
CTCGAGTTAATGAAAATCTCCTGGGTCAAAAGACCCGATACGGTTACACTGGTCGGA
TGGCTAAGTCCTCCCTGCCTCTTTTTGACGGCCTCATCAAGTATGACCTGAACAATG
GCAAGAGCCAGACTCATGAATTCGGTTCTGGTCGATATGGAGGAGAAGCCGTGTTT
GTGCCTCGACCCGGTGCAACTGCTGAGGACGACGGCTGGCTGGTTACCTTCGTCCAC
GATACTGCTAAAGACACTTCCGAACTCGTGGTCGTGTCGGCACAGGACGTCACCGGT
GAGCCCGTCGCTAGAGTGCTTATCCCCCAGCGAGTTCCTTACGGTTTTCATGGCGCA
TGGGTGTCTGAGGAGCAACTGAAGGCATCGATGTGA
Calothrix sp. NIES-2098 carotenoid oxygenase
Amino acid sequence of the protein BAY08575.1 (SEQ ID NO: 20):
MTTNTIPTTAINPYLDGNFAPVREEITTDTLQVVGQLPPDLAGMFVRNGPNPQWPPIGKY
HWFDGDGMLHGVRISNGQATYRNRYIQTRGWKIEREAGKAIWTGFMEPPRMDNPYGG
YKNTGNTALIWHAGQLLALNEGGAPHAMTLPELGTIGEYTYDGKLISAFTAHPKVDPVT
GEMMFFGYSFAPPYLQYSQVSAAGELLQTVPIDLPTPVMMHDFAITENYTIFMDLPLTLS
AERLQRGEPMLMFERDRSSRFGIVPRYGDNSNIRWFESSPCYVFHTLNAYEVGDEVVLI
GCRMSSTSVLVTEDNQTDADGNIPRLHQWRFNLKTGTVREQQLDDLAAEFPRVNEDFL
GRQTRYGYAGKLANSPLPLFDGVIKYDLNGETSQIHHFGQGRYGGEAVFAPRPGATHE
DDGWLITFVYDEGSDTSELVVINAQDITSEPVARVIIPQRVPYGFHGTWVSEEQLMTNS
Codon-optimized nucleotide sequence of BAY08575.1 for
Yarrowia lipolytica (SEQ ID NO: 21):
ATGACCACAAATACGATCCCCACTACCGCCATCAACCCCTATCTTGACGGCAATTTC
GCACCTGTGCGGGAAGAAATCACTACGGACACCCTTCAAGTTGTTGGTCAACTCCCT
CCTGATCTGGCCGGCATGTTCGTGCGGAATGGTCCCAATCCCCAGTGGCCTCCTATC
GGTAAATATCACTGGTTCGATGGTGACGGCATGCTCCATGGAGTGCGTATCTCGAAT
GGTCAGGCAACCTATCGGAACCGATACATCCAAACCCGTGGATGGAAAATTGAGCG
TGAAGCCGGCAAAGCTATCTGGACTGGATTTATGGAGCCTCCTCGAATGGACAATCC
TTATGGTGGATATAAAAACACGGGCAATACAGCTCTGATTTGGCATGCAGGTCAGCT
TCTGGCACTTAACGAAGGTGGAGCCCCTCACGCTATGACTCTTCCTGAGCTCGGAAC
CATCGGAGAATACACCTATGACGGTAAACTCATCTCCGCATTCACTGCTCACCCCAA
GGTGGATCCTGTTACCGGCGAGATGATGTTCTTTGGATACTCCTTCGCACCTCCTTAT
CTTCAATACAGCCAGGTGTCTGCAGCAGGCGAGCTCCTTCAAACTGTGCCTATCGAT
CTCCCTACGCCTGTGATGATGCATGACTTTGCCATCACTGAGAACTACACAATCTTT
ATGGACCTTCCCCTGACGCTTTCGGCAGAGCGTCTTCAAAGAGGCGAACCTATGCTT
ATGTTCGAACGAGATCGATCGAGCCGGTTCGGTATCGTTCCCCGTTATGGTGATAAT
TCGAATATTCGTTGGTTTGAGTCCTCTCCCTGCTACGTGTTTCATACACTGAATGCCT
ATGAGGTGGGAGACGAGGTCGTGCTTATTGGATGCCGGATGAGCTCCACCTCCGTGC
TTGTGACGGAGGACAATCAGACCGACGCAGACGGTAATATTCCCAGACTTCACCAA
TGGAGATTTAATCTCAAGACTGGTACAGTTCGGGAGCAGCAGCTCGACGATCTCGCC
GCCGAATTTCCCCGAGTTAACGAGGACTTCCTCGGAAGACAAACTCGGTACGGCTAT
GCTGGAAAACTGGCTAACTCGCCCCTGCCTCTGTTTGACGGAGTTATCAAATATGAC
CTCAACGGCGAGACTTCCCAGATCCACCATTTTGGACAGGGAAGATACGGCGGCGA
AGCCGTTTTTGCCCCCAGACCTGGTGCCACCCATGAAGACGACGGATGGCTCATCAC
ATTTGTCTACGACGAGGGATCCGACACGAGCGAGCTCGTGGTCATCAACGCTCAAG
ATATTACGTCCGAACCCGTTGCTCGTGTGATTATTCCCCAACGTGTGCCCTACGGATT
TCACGGTACTTGGGTCAGCGAAGAACAGCTCATGACAAACAGCTAA
Calothrix brevissima NIES-22 carotenoid oxygenase
Amino acid sequence of the protein BAY62420.1 (SEQ ID NO: 22):
MTSNIISTTADNPYLSGNFAPVGQEIATDSLPVLGELPPDLSGMFVRNGPNPQWPPIGNY
HWFDGDGMLHCVHVSEGKATYRNRYVQTQGWKIEREAGKAVWSGLLEPPQMDNPYG
AYKRTANTALIWHGGQMLALHEGSAPHAIKVPELETIGEYTYNDKLVSAFTAHPKVDP
VTGEMMFFGYSFTPPYLQYSVVSAVGELLQTVPIDLPVAVMMHDFAITKNYSIFLDLPLT
MRGERLQRGEPLFMFERDRPSRFGIIPRYGNNSNIRWFESEPCYIFHIYNAYEIGDEVVLL
GCRMSSTTVLGDDNSSDPDANVPRLHEWRFNLKTGTVSDQRLDDIPAEFPRINTENYVGL
PTRYGYAGKAANTPVPLFDGLIKYDFSSGKSQTHEFGQGRFGGEAVFAPRPGATAEDDG
WLITFVHDETS DT SELLVVHAQDVSSEPVARVIIPQRVPYGFHGAWISEAQLKGA
Codon-optimized nucleotide sequence of BAY62420.1 for
Yarrowia lipolytica (SEQ ID NO: 23):
ATGACTAGCAATATCATTTCCACTACAGCTGATAATCCCTACCTGTCCGGAAATTTC
GCTCCTGTCGGCCAGGAAATCGCCACAGATTCCCTCCCCGTCCTGGGTGAACTCCCT
CCCGATCTGAGCGGTATGTTCGTCCGGAATGGACCCAACCCCCAATGGCCTCCTATC
GGAAACTATCATTGGTTCGACGGAGATGGTATGCTGCACTGTGTTCACGTTTCGGAA
GGAAAAGCCACTTATAGAAATCGTTATGTGCAGACCCAGGGTTGGAAAATTGAGCG
GGAAGCTGGAAAGGCTGTTTGGTCGGGTCTGCTCGAACCTCCTCAGATGGACAATCC
TTACGGTGCATATAAAAGAACCGCTAATACAGCACTGATTTGGCACGGAGGTCAAA
TGCTCGCACTCCATGAGGGATCTGCACCCCACGCTATTAAGGTCCCCGAGCTGGAAA
CGATCGGCGAGTACACATATAATGATAAGCTGGTGTCGGCTTTTACGGCACATCCCA
AAGTGGATCCCGTGACGGGCGAGATGATGTTCTTTGGCTACTCCTTTACACCCCCCT
ATCTGCAATATTCGGTTGTGAGCGCTGTTGGTGAGCTGCTTCAGACTGTGCCCATCG
ATCTCCCCGTCGCCGTTATGATGCATGACTTTGCCATCACTAAAAACTACAGCATTTT
CCTGGATCTTCCTCTTACTATGCGTGGAGAGCGTCTTCAACGGGGAGAACCTCTTTTT
ATGTTTGAGCGGGACCGTCCTTCTCGATTTGGTATTATCCCTCGTTATGGTAATAATT
CTAATATTCGTTGGTTTGAAAGCGAACCTTGCTATATTTTCCACATCTACAACGCTTA
TGAAATCGGAGACGAGGTCGTGCTCCTGGGTTGTCGAATGAGCTCTACCACCGTTCT
GGGAGATGACAACTCGTCGGACCCCGACGCCAATGTTCCTCGACTTCATGAGTGGA
GATTTAATCTGAAGACGGGAACGGTGTCTGATCAACGACTTGACGACATTCCTGCTG
AATTCCCTCGAATCAACGAGAATTATGTCGGCCTCCCCACCAGATATGGCTACGCAG
GAAAGGCCGCAAACACGCCTGTCCCCCTCTTCGATGGACTTATTAAGTATGATTTTT
CGTCGGGTAAGAGCCAAACCCATGAATTTGGACAGGGCCGTTTTGGCGGCGAAGCT
GTCTTCGCACCCAGACCTGGTGCTACTGCTGAAGATGATGGATGGCTCATCACCTTT
GTCCACGACGAAACGTCTGACACCTCCGAACTTCTTGTCGTTCACGCTCAGGATGTG
TCTTCTGAGCCCGTTGCCCGAGTCATCATTCCTCAGCGGGTCCCCTACGGTTTCCACG
GAGCATGGATTTCTGAAGCTCAACTGAAAGGAGCCTAA
Scytonema millet 9-cis-epoxycarotenoid dioxygenase
Amino acid sequence of the protein WP_069349917.1
(SEQ ID NO: 24):
MATTQVNPYLDGNFAPVREESTANTLQVIGELPPDLSGMFVRNGPNPQWTPIGKYHWF
DGDGMLHGVRISNGKATYRNRYVRTKKWKIENEAGKALLSGLLEPPQKKNPPGASKNT
ANTALVWHAGQLLALWEGGAPHAIRVPELKTKGEYTYNGKLASAFTAHPKVDPVTGE
MMFFGYGFAPPYLQYSVVSPEGELLQTEPIDIPMAVMMHDFAITQDYTIFMDLPLTFSQE
RRKRGEPMMKFERDKPSRFGIVPRYGNNSNIRWFESPACYIFHTLNAYEEGDEVVLIACR
MNSTTVLDVPQDTHTDSEADIPRLHRWRFNLKTGKVSEEMLDDTASEFPRINENLLGQK
TRYGYTGKMAKSSMPLFDGLIKYDFNTGKSQTHEFGRGRYGGEAVFVPRPGATAEDDG
WLVTFVHDTVEETSELVVVSAQDITGEPVARVLIPQRVPYGFHGAWVSEE QLKASV
Codon-optimized nucleotide sequence of WP_069349917.1
for Yarrowia lipolytica (SEQ ID NO: 25):
ATGGCAACTACCCAGGTTAACCCCTACCTGGACGGAAACTTCGCCCCCGTGAGAGA
AGAGAGCACCGCTAATACACTGCAGGTCATCGGAGAGCTTCCCCCTGATCTCTCGGG
CATGTTCGTTAGAAATGGTCCCAACCCTCAATGGACACCTATCGGTAAGTATCACTG
GTTTGACGGCGATGGCATGCTCCACGGCGTGCGTATCAGCAATGGCAAAGCTACCTA
TAGAAATCGGTACGTTCGGACCAAAAAGTGGAAGATTGAGAATGAAGCAGGAAAA
GCTCTCCTTTCTGGCCTTCTTGAACCCCCTCAAAAGAAAAACCCCCCTGGCGCTTCC
AAGAACACCGCCAATACCGCCCTTGTGTGGCATGCCGGACAACTTCTGGCTCTTTGG
GAAGGTGGTGCACCTCACGCAATTAGAGTCCCTGAGCTCAAGACGAAAGGTGAGTA
TACGTACAATGGAAAGCTGGCCAGCGCCTTTACAGCCCATCCCAAGGTTGATCCTGT
CACAGGAGAGATGATGTTTTTTGGTTACGGCTTTGCCCCTCCTTATCTGCAATATTCG
GTCGTTTCTCCCGAAGGCGAACTGCTGCAAACAGAGCCCATTGACATTCCTATGGCC
GTCATGATGCACGATTTTGCCATTACCCAGGATTACACCATTTTTATGGACCTGCCCC
TTACGTTTTCTCAGGAACGTCGGAAACGAGGTGAGCCCATGATGAAATTCGAGCGG
GATAAACCCTCGCGTTTCGGAATTGTCCCCAGATACGGAAATAATAGCAACATCCGT
TGGTTTGAAAGCCCTGCTTGTTACATTTTCCATACCCTCAACGCATACGAAGAAGGT
GACGAGGTGGTTCTTATCGCTTGCCGGATGAACTCGACAACAGTGCTTGATGTCCCT
CAAGACACCCATACCGATAGCGAGGCTGATATCCCCCGACTGCATCGATGGCGTTTT
AACCTTAAGACTGGAAAAGTCTCGGAAGAGATGCTTGATGACACGGCATCCGAATT
TCCTCGAATTAATGAAAACCTTCTGGGTCAGAAAACACGATATGGATACACCGGCA
AAATGGCTAAAAGCAGCATGCCTCTTTTCGACGGCCTCATTAAGTATGACTTTAACA
CGGGTAAATCCCAGACACACGAATTTGGACGTGGACGATATGGTGGTGAAGCCGTG
TTCGTCCCTCGACCTGGTGCCACCGCCGAGGATGATGGTTGGCTTGTCACGTTTGTCC
ACGACACTGTCGAGGAAACGTCGGAGCTCGTTGTCGTGTCTGCACAGGACATCACTG
GTGAACCCGTGGCCAGAGTCCTCATTCCTCAGCGGGTTCCCTATGGCTTCCATGGAG
CTTGGGTGAGCGAAGAACAGCTGAAGGCCTCGGTTTGA
Lactuca sativa lycopene epsilon-cyclase
Codon-optimized nucleotide sequence of Lactuca sativa
lycopene epsilon-cycalse for Yarrowia lipolytica
(SEQ ID NO: 26):
ATGAAGTGCTCCGCGAAGTCCGATCGTTGCGTGGTGGATAAGCAGGGTATCTCCGTG
GCCGACGAAGAGGATTACGTCAAGGCCGGAGGCTCCGAACTTTTCTTCGTCCAGATG
CAGCGGACCAAGTCTATGGAGTCACAGAGCAAGTTGAGCGAAAAGCTCGCTCAGAT
TCCAATCGGAAACTGCATTTTGGATTTGGTTGTCATCGGCTGCGGCCCAGCTGGCCT
CGCTTTGGCGGCGGAATCGGCAAAGCTTGGCCTCAACGTGGGCCTGATCGGTCCAG
ATCTGCCATTCACCAACAACTACGGTGTGTGGCAGGACGAGTTCATTGGCCTGGGTC
TTGAAGGCTGCATCGAACACTCTTGGAAGGACACCCTCGTCTACCTGGATGACGCCG
ATCCAATCCGCATTGGACGTGCTTACGGACGCGTTCACCGTGACCTCCTGCACGAAG
AGCTGCTGCGTCGCTGCGTTGAGTCCGGCGTGTCTTACCTCAGCAGCAAGGTTGAAC
GCATCACCGAAGCACCAAACGGCTACTCACTCATCGAGTGCGAAGGTAACATCACC
ATCCCTTGCCGCCTCGCAACCGTGGCTTCGGGCGCCGCCTCCGGCAAGTTCCTGGAG
TACGAGTTGGGCGGACCTCGCGTGTGCGTGCAGACCGCGTACGGCATCGAGGTTGA
GGTCGAAAACAACCCATACGACCCTGATCTGATGGTTTTCATGGACTACCGTGACTT
CTCGAAGCACAAGCCTGAATCCCTTGAGGCAAAGTACCCAACTTTCCTTTACGTCAT
GGCTATGTCCCCAACCAAGATCTTCTTCGAGGAAACTTGCCTCGCTTCTCGCGAAGC
TATGCCTTTCAACTTGTTGAAGAGCAAGCTGATGTCCCGCCTGAAGGCTATGGGCAT
CCGCATCACCCGCACCTACGAGGAAGAATGGTCGTACATCCCGGTGGGCGGCAGCC
TGCCAAACACCGAGCAGAAGAACCTCGCCTTCGGTGCAGCAGCCTCGATGGTGCAC
CCAGCCACCGGTTACTCTGTGGTTCGCTCTCTGAGCGAAGCACCAAACTACGCCGCC
GTTATTGCGAAGATCCTGCGCCAGGACCAGTCAAAGGAGATGATTTCCCTCGGTAAG
TACACTAACATCTCCAAGCAGGCCTGGGAAACTCTCTGGCCACTTGAGCGCAAGCG
CCAGCGTGCCTTCTTCCTCTTCGGCTTGTCACACATCGTGCTCATGGACCTGGAAGG
CACTCGCACTTTCTTCCGTACCTTCTTCCGCCTGCCTAAGTGGATGTGGTGGGGCTTC
CTGGGCTCTTCGCTTTCCTCCACCGATTTGATTATCTTCGCTCTCTACATGTTCGTCAT
CGCTCCGCACTCCTTGCGTATGGAGCTCGTCCGTCACCTGTTGTCCGATCCGACCGG
TGCCACTATGGTGAAGGCATACCTGACCATCTAA
E. coli Thioredoxin (TrxA) tag
Amino acid sequence of TrxA (SEQ ID NO. 27):
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
Codon-optimized nucleotide sequence of trxA gene for
Yarrowia lipolytica (SEQ ID NO: 28):
ATGAGCGATAAAATTATTCACCTGACTGACGACAGTTTTGACACGGATGTACTCAAA
GCGGACGGGGCGATCCTCGTCGATTTCTGGGCAGAGTGGTGCGGTCCGTGCAAAAT
GATCGCCCCGATTCTGGATGAAATCGCTGACGAATATCAGGGCAAACTGACCGTTGC
AAAACTGAACATCGATCAAAACCCTGGCACTGCGCCGAAATATGGCATCCGTGGTA
TCCCGACTCTGCTGCTGTTCAAAAACGGTGAAGTGGCGGCAACCAAAGTGGGTGCA
CTGTCTAAAGGTCAGTTGAAAGAGTTCCTCGACGCTAACCTGGCG
Conserved domain in Zea mays oleosin
Amino acid sequence of conserved domain in Zea mays
oleosin (SEQ ID NO. 29):
MAVAKALATATAAFSMLLLSGLAVTGTVLALIVATPLMVIFSPVLVPAAITVALLTVGIV
SSGGFGVAAVAVLAWVYRYL
Conserved domain in Sesamum indicum oleosin
Amino acid sequence of conserved domain in Sesamum indicum
oleosin (SEQ ID NO. 30):
TQILAIITLLPISGTLLCLAGITLVGTLIGLAVATPVFVIFSPVLVPAAILIAGAVTA
FLTSGAFGLTGLSSLSWVLNSF

1. A recombinant microbial production host cell for producing an ionone compound, said host cell comprising at least one nucleic acid construct comprising a first coding sequence, wherein said first coding sequence encodes a fusion enzyme comprising a first domain capable of functioning as a lipid body compartmentalization signal tag fused to a second domain having carotenoid cleavage activity.

2. The host cell of claim 1, wherein the first domain is selected from the group consisting of a lipid body structural protein, a lipid synthesis enzyme, a membrane protein, and a carotenoid-binding protein.

3. The host cell of claim 1, wherein the first domain comprises an amino acid sequence having at least 80% sequence identity to SEQ ID NO: 1, SEQ ID NO: 3, SEQ ID NO: 5, SEQ ID NO: 7, or SEQ ID NO: 9.

4. (canceled)

5. The host cell of claim 3, wherein the first domain is an oleosin polypeptide comprising the amino acid sequence of SEQ ID NO: 29 or SEQ ID NO: 30.

6. The host cell of claim 1, wherein the second domain is a carotenoid cleavage dioxygenase or a carotenoid oxygenase.

7. The host cell of claim 6, wherein the second domain comprises an amino acid sequence exhibiting at least 80% sequence identity to SEQ ID NO: 11, SEQ ID NO: 14, SEQ ID NO: 16, SEQ ID NO: 18, SEQ ID NO: 20, SEQ ID NO: 22, or SEQ ID NO: 24.

8.-12. (canceled)

13. The host cell of claim 1 for producing beta-ionone, further comprising in the same nucleic acid construct or a different nucleic acid construct a second coding sequence encoding a phytoene synthase.

14. The host cell of claim 13, further comprising in the same nucleic acid construct or a different nucleic acid construct a third coding sequence encoding a phytoene dehydrogenase.

15. The host cell of claim 1, further comprising one or more nucleic acid constructs comprising one or more coding sequences, wherein said one or more coding sequences are selected from mevalonate pathway enzymes native to the host cell.

16.-17. (canceled)

18. The host cell of claim 1, wherein the host cell is a yeast.

19.-20. (canceled)

21. A recombinant microbial production host cell for producing a carotenoid compound, said host cell comprising at least one nucleic acid construct comprising a coding sequence encoding a fusion enzyme comprising an oleosin polypeptide fused to a beta-carotene ketolase or a beta-carotene hydroxylase.

22. The host cell of claim 21 comprising a first coding sequence encoding a first fusion enzyme comprising an oleosin polypeptide fused to a beta-carotene ketolase and a second coding sequence encoding a second fusion enzyme comprising an oleosin polypeptide fused to a beta-carotene hydroxylase.

23.-24. (canceled)

25. The host cell of claim 21, wherein the beta-carotene hydroxylase is an algal CrtR-B.

26.-27. (canceled)

28. The host cell of claim 21, wherein the carotenoid compound is astaxanthin or canthaxanthin.

29. (canceled)

30. The host cell of claim 21, wherein the host cell is Yarrowia lipolytica.

31. A synthetic or recombinant nucleic acid molecule comprising a first nucleic acid sequence and a second nucleic acid sequence, wherein the first nucleic acid sequence and the second nucleic acid sequence together encode a fusion enzyme, said fusion enzyme comprising a first domain capable of functioning as a lipid body compartmentalization signal tag and a second domain having carotenoid 9,10(9′,10′)-cleavage activity.

32. The nucleic acid molecule of claim 31, wherein the first domain is selected from the group consisting of a lipid body structural protein, a lipid synthesis enzyme, a membrane protein, and an orange carotenoid binding protein.

33.-38. (canceled)

39. The nucleic acid molecule of claim 31, wherein the second domain is a carotenoid 9,10(9′,10′)-cleavage dioxygenase, a carotenoid oxygenase, or a 9-cis-epoxycarotenoid dioxygenase.

40.-52. (canceled)

53. A fusion enzyme comprising a lipid body compartmentalization signal tag (LBT) domain coupled to a carotenoid cleavage dioxygenase (CCD) domain.

54.-64. (canceled)

65. A method for producing an ionone compound, wherein the method comprises culturing a recombinant host cell of claim 1 in a medium comprising glucose or glycerol under conditions whereby an ionone compound is produced.

66.-67. (canceled)

68. A method for producing a carotenoid compound, wherein the method comprises culturing a recombinant host cell of claim 21 in a medium comprising glucose or glycerol under conditions whereby a carotenoid compound is produced.

69.-70. (canceled)
